SEQUENCE LISTING (7 sequences)

SEQ ID NO: 1 - protein, 12 aa, Artificial Sequence
  Description of Artificial Sequence Synthetic peptide
CYGRKKRRQRRR

SEQ ID NO: 2 - protein, 10 aa, Artificial Sequence
  Description of Artificial Sequence Synthetic peptide
CRRRRRRRRR

SEQ ID NO: 3 - DNA, 16 bp, Artificial Sequence
  Description of Artificial Sequence Synthetic oligonucleotide
ctcaatccatggcagc

SEQ ID NO: 4 - DNA, 21 bp, Artificial Sequence
  Description of Combined DNA/RNA Molecule Synthetic oligonucleotide
gcaugcggccucuguuugatt

SEQ ID NO: 5 - DNA, 21 bp, Artificial Sequence
  Description of Combined DNA/RNA Molecule Synthetic oligonucleotide
ucaaacagaggccgcaugctt

SEQ ID NO: 6 - DNA, 18 bp, Artificial Sequence
  Description of Artificial Sequence Synthetic oligonucleotide
tctcccagcgtgcgccat

SEQ ID NO: 7 - DNA, 16 bp, Artificial Sequence
  Description of Artificial Sequence Synthetic oligonucleotide
tggcaagcatcctgta

(19) United States
(12) Patent Application Publication
(10) Pub. No.: US 2009/0202573 A1
(43) Pub. Date: Aug. 13, 2009

(54) POLYMERIC CONJUGATES CONTAINING POSITIVELY-CHARGED MOIETIES

(75) Inventors: Hong ZHAO, Edison, NJ (US); Prasanna REDDY, Somerset, NJ (US); Ivan HORAK, West Orange, NJ (US); Jing XIA, Warren, NJ (US)

Correspondence Address: LUCAS & MERCANTI, LLP, 475 PARK AVENUE SOUTH, 15TH FLOOR, NEW YORK, NY, 10016, US

(73) Assignee: ENZON PHARMACEUTICALS, INC., Bridgewater, NJ (US)

(21) Appl. No.: 12/402,922

(22) Filed: Mar. 12, 2009

Related U.S. Application Data
(63) Continuation of application No. PCT/US07/78598, filed on Sep. 15, 2007.
(60) Provisional application No. 60/844,944, filed on Sep. 15, 2006.
(60) Provisional application No. 60/844,945, filed on Sep. 15, 2006.
(60) Provisional application No. 60/861,349, filed on Nov. 27, 2006.
(60) Provisional application No. 60/861,350, filed on Nov. 27, 2006.
(60) Provisional application No. 60/911,734, filed on Apr. 13, 2007.
(60) Provisional application No. 60/956,814, filed on Aug. 20, 2007.

(51) Int. Cl. A61K 47/48; C07C 271/22; C07D 403/14; C07H 21/02; C07K 16/00; A61K 31/27; A61K 31/4025; A61K 38/00; A61K 31/7105; A61K 31/496; A61K 39/395; A61K 38/43; C12N 5/00

(52) U.S. Cl. 424/179.1; 560/148; 548/520; 530/328; 530/300; 536/23.1; 530/327; 544/357; 435/183; 530/391.1; 514/483; 514/425; 514/2; 514/252.11; 424/94.1; 435/375

(57) ABSTRACT

The present invention provides polymeric conjugates containing positively charged moieties. Methods of making the polymeric delivery systems and methods of treating mammals using the same are also disclosed.

### CROSS-REFERENCE TO RELATED APPLICATION

This application claims the benefit of priority from U.S. Provisional Patent Application Ser. Nos. 60/844,944 filed Sep. 15, 2006, 60/844,945 filed Sep. 15, 2006, 60/861,349 filed Nov. 27, 2006, 60/861,350 filed Nov. 27, 2006, 60/911,734 filed Apr. 13, 2007 and 60/956,814 filed Aug. 20, 2007, the contents of each of which are incorporated herein by reference.

### BACKGROUND OF THE INVENTION

In the past it has been determined that it would be beneficial to increase the positive charge of polymers or conjugates containing the same when used in the delivery of biologically active moieties such sa proteins, peptides and the like. For example, commonly assigned U.S. Pat. No. 5,730,990 describes PEG and related polyalkylene oxides having a single secondary or tertiary amine group attached thereto. The purpose of the combination was to allow the amine-derived polymers to impart a pI and/or pH modulating effect to the conjugate. Thus, the isoelectric point of bioactive materials included in the conjugate could be adjusted to a desired point. The aforementioned '990 patent offered a solution to counteract the effect observed with conventional activated polymers where shifts in isoeletric points were observed, often to the detriment of optimal activity.

Over the years, some oligonucleotide based therapeutics have benefited from several advances exemplified by the discovery and development of RNA interference and microRNA, as well as improvements in composition design such as the use of locked nucleic acid (LNA) structural backbones, Short interfering RNA (siRNA) has evolved from a research tool to therapeutic agent in clinical trials within just a few years. However, in vivo delivery is still the major hurdle to fully realize the therapeutic potential for oligonucleotide-based therapies. Presently, direct intra-compartmental injection and continuous infusion are still the major routes of administration. As a consequence, improvements in drug delivery technology have been sought for the field of oligonucleotides used for therapeutic purposes.

Due to the highly negatively-charged backbone of oligonucleotides, it is often difficult for them to cross the cellular membrane and exhibit their biological activity. The negative charges prevent the oligonucleotides from approaching negatively-charged cell membrane and thus reduce endocytosis. In the past, oligonucleotides have been attached or complexed with positively-charged peptides, cationic lipids or cationic polymers to address this issue. The results have not been completely satisfactory. Thus, further improvements were desired. The present invention addresses this need and others.

### SUMMARY OF THE INVENTION

In order to overcome the above problems and improve the technology for drug delivery, there are provided new polymeric delivery systems containing positively-charged backbones.

In one aspect of the present invention, there are provided compounds of Formula (I):

{Z₂}_{b}-R₁-{Z₁}ₐ

wherein

each Z₁ is independently

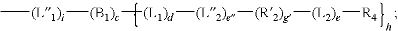

each Z₂ is independently selected capping groups,

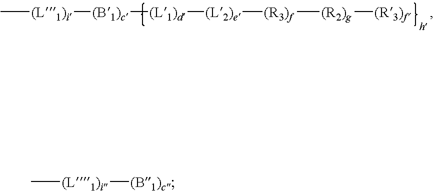

R₁ is a substantially non-antigenic polymer,

R₂ and R′₂ are independently selected positive charge-containing peptides or nitrogen-containing cyclohydrocarbon moieties;

R₃ and R′₃ are independently selected targeting agents;

R₄ is a biologically active moiety;

B₁, B′₁ and B″₁ are independently selected branching groups;

L₁, L′₁, L″, L₁′″ and L₁″″ are independently selected bifunctional linkers;

L₂, L′₂ and L″₂ are independently selected releaseable linkers;

(a) is a positive integer, preferably from 1 to about 31, more preferably from about 3 to about 8, and most preferably 1;

(b) is zero or a positive integer, preferably from about 0 to about 31, more preferably from about 3 to about 7;

(c), (c′) and (c″) are independently zero or a positive integer, preferably zero, 1, 2 or 3, and more preferably zero or 1;

(d), (d′), (i), (i′) and (i″) are independently zero or a positive integer, preferably zero, 1, 2 or 3, and more preferably zero or 1;

(e) is a positive integer, preferably 1, 2 or 3, and more preferably 1 or 2;

(e′) and (e″) are independently zero or a positive integer, preferably zero, 1, 2 or 3, and more preferably zero, 1 or 2;

(f) and (f′) are independently zero or a positive integer, preferably zero, 1, or 2, and more preferably zero or 1;

(g) is a positive integer, preferably from about 1 to about 5, and more preferably 1 or 2;

(g′) is zero or a positive integer, preferably 0 or an integer from about 1 to about 5, and more preferably zero, 1 or 2; and

(h) and (h′) are independently selected positive integers, preferably from about 1 to about 8, more preferably 1, 2, 3 or 4, and most preferably 1 or 2;

provided that (g′) is a positive integer when (b) is not zero and all Z₂ are capping groups, -(L″″₁)_{i″}-(B″₁)_{c″} or in combination.

In one preferred aspect of the polymeric compounds, the sum of (a) and (b) equals to from about 1 to about 32.

In some preferred embodiments, the polymeric compounds can include four-arm, 8 arm, 16 arm and 32 arm polymers as will be described and illustrated below. More preferably, four armed polymers can be employed with a branching moiety at each terminal of the polymer arms. The polymeric compounds containing four arms and a branching moiety thereon can have up to 8 functional sites to load positively-charged moieties and/or biologically active moieties.

In another preferred embodiment, the multi-arm polymeric compounds described herein contain one polymer terminal bonded to a biologically active moiety and each of the other polymer terminals bonded to a positive charge-containing moiety.

In another aspect, the polymeric compounds described herein contain positively-charged peptides and piperazine-based moieties, for example. The positive charge-containing moieties are capable of conferring additional positive charges to the substantially non-antigenic polymer.

In another aspect, the positively charged peptides can help the polymeric compounds penetrate cell membrane. The preferred positively-charged peptides can be cell-membrane penetrating peptides (CPPs) such as TAT, for example.

In yet another aspect of the invention, there are provided polymeric conjugates containing positively-charged backbones to neutralize the negatively charged biologically active molecules and improve the cellular uptake of biologically active moieties such as oligonucleotides, locked nucleic acid (LNA), short interfering RNA (siRNA), aptamer, ribozyme, DNA decoy, etc.

In yet another aspect of the invention, the biologically active moieties are attached to the polymeric portion of the compounds described herein via releasable linkers. Among the releasable linkers can be benzyl elimination-based linkers, trialkyl lock-based linkers, bicine-based linkers, a disulfide bond, hydrazone-containing linkers and thiopropionate-containing linkers. Alternatively, the releasable linkers are intracellular labile linkers, extracellular linkers and acidic labile linkers.

In yet another aspect of the invention, the positively-charged moieties and targeting agents can be linked to the polymeric portion of the compounds described herein via permanent linkers and releasable linkers alone or in combination. Preferably, the positively-charged peptides and targeting agents are linked via permanent linkers. Targeting agents such as RGD peptide, folic acid, single chain antibody (SCA), etc. can be attached to the polymeric compound described herein to guide the conjugate to the tissue of interest in vivo. The design provides a novel approach for the targeted delivery of negatively-charged molecules such as oligonucleotides in vivo and enhances the cellular uptake of these molecules to have better therapeutic efficacies.

In some preferred aspects of the invention, the positively-charged peptide can be also therapeutic peptides specific to targeted, affected regions such as NGR, TNFα and TAT. Artisan of ordinary skill can employ various therapeutic peptides containing positive charges and capable of being delivered specific to targeted area.

In other aspects, the cell penetrating peptides can be replaced with one of a variety of positively charged targeting peptides like TAT, RGD-TAT and NGR, for example for targeted delivery to the tumor site.

When the PEG linkers with positively-charged backbone are conjugated with negatively-charged therapeutic molecules such as oligonucleotides, the negative charge of oligonucleotides can be neutralized and the net charge of the conjugates can be positive. The overall shape of the PEG conjugates can be spherical when multi-arm PEG is used. Due to the property that PEG is highly hydrated in aqueous solution, the multi-arm PEG conjugates with positively-charged backbone appear as spherical “mini-nanoparticles” with oligonucleotides embedded in the center.

The positively-charged moieties capable of neutralizing negatively-charged oligonucleotides can reduce toxicity and also facilitate penetrating cell membranes thereof and thereby improve the delivery of oligonucleotides. As a result, highly negatively-charged oligonucleotides can be delivered in vivo with less toxicity.

One advantage of the polymer conjugates of the invention is that cellular uptake is improved by attaching highly positively charged peptides and cell penetrating peptides like TAT. Moreover, the artisan can achieve targeting function by attaching targeting peptides, aptamers and folates etc.

Another advantage is that the release rates/sites of the negatively charged molecules from the prodrugs can be modified. The drugs attached to the polymeric compounds described herein can be released at modified rates, thus allowing the artisan to achieve desired bioavailability of therapeutic peptides and oligonucleotides. The site of release of the negatively-charged therapeutic agents can be also modified, i.e. release at different compartments of cells. Thus, the polymeric delivery systems described herein allow sufficient amounts of the negatively-charged therapeutic agents to be available selectively at the desired target area, i.e. macropinosome and endosome. The temporal and spatial modifications alone and in combination of release of the therapeutic agents can be advantageous for treatment of disease.

The polymeric compounds with positive backbone are stable under buffer conditions and the oligonucleotides or other therapeutic agents are not prematurely excreted from the body.

A still further advantage of the present invention is that the conjugates described herein allow significantly improved cellular uptake and specific mRNA down regulation in cancer cells in the absence of transfection agents. This technology can be applied to the in vivo administration of oligonucleotide drugs. For example, cellular uptake of the PEG-oligonucleotides including antisense Bcl2 oligonucleotides, Bcl2 siRNA or anti Survivin LNA described herein was greater than that of native antisense Bcl2 oligonucleotides or Bcl2 siRNA by human lung cancer cells without transfection agents. Moreover, the conjugates described herein allowed higher cellular uptake in the absence of transfection agent compared to that aided by transfection agents.

Other and further advantages will be apparent from the following description.

For purposes of the present invention, the term “residue” shall be understood to mean that portion of a compound, to which it refers, i.e. PEG, oligonucleotide, etc. that remains after it has undergone a substitution reaction with another compound.

For purposes of the present invention, the term “polymeric residue” or “PEG residue” shall each be understood to mean that portion of the polymer or PEG which remains after it has undergone a reaction with other compounds, moieties, etc.

For purposes of the present invention, the term “alkyl” shall be understood to include straight, branched, substituted, e.g. halo-, alkoxy-, nitro-, C₁₋₁₂, but preferably C₁₋₄ alkyls, C₃₋₈ cycloalkyls or substituted cycloalkyls, etc.

For purposes of the present invention, the term “substituted” shall be understood to include adding or replacing one or more atoms contained within a functional group or compound with one or more different atoms.

For purposes of the present invention, substituted alkyls include carboxyalkyls, aminoalkyls, dialkylaminos, hydroxyalkyls and mercaptoalkyls; substituted alkenyls include carboxyalkenyls, aminoalkenyls, dialkenylaminos, hydroxyalkenyls and mercaptoalkenyls; substituted alkynyls include carboxyalkynyls, aminoalkynyls, dialkynylaminos, hydroxyalkynyls and mercaptoalkynyls; substituted cycloalkyls include moieties such as 4-chlorocyclohexyl; aryls include moieties such as napthyl; substituted aryls include moieties such as 3-bromo phenyl; aralkyls include moieties such as tolyl; heteroalkyls include moieties such as ethylthiophene; substituted heteroalkyls include moieties such as 3-methoxy-thiophene; alkoxy includes moieties such as methoxy, and phenoxy includes moieties such as 3-nitrophenoxy. Halo shall be understood to include fluoro, chloro, iodo and bromo.

For purposes of the present invention, “nucleic acid”, “nucleotide” or “oligonucleotide” shall be understood to include deoxyribonucleic acid (DNA), ribonucleic acid (RNA) whether single-stranded or double-stranded, unless otherwise specified, and any chemical modifications thereof.

For purposes of the present invention, “positive integer” shall be understood to include an integer as will be understood by those of ordinary skill to be within the realm of reasonableness by the artisan of ordinary skill.

The terms “effective amounts” and “sufficient amounts” for purposes of the present invention shall mean an amount which achieves a desired effect or therapeutic effect as such effect is understood by those of ordinary skill in the art.

### BRIEF DESCRIPTION OF THE DRAWINGS

FIG. 1 schematically illustrates methods of synthesis described in Examples 1-3.

FIG. 2 schematically illustrates methods of synthesis described in Examples 4-13.

FIG. 3 schematically illustrates methods of synthesis described in Examples 14-20.

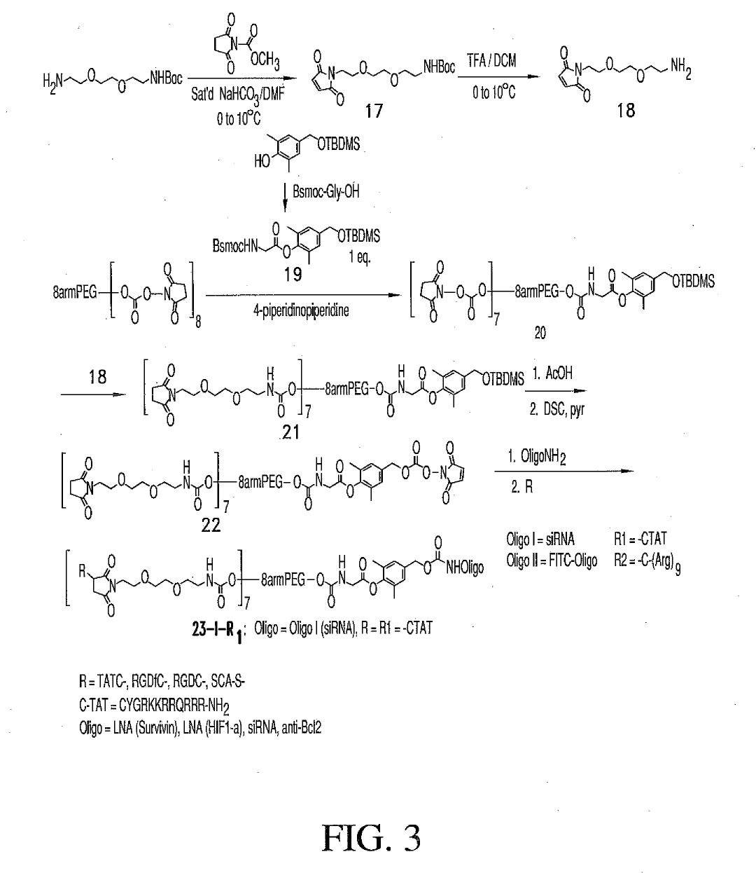

FIG. 4 schematically illustrates methods of synthesis described in Examples 21-26.

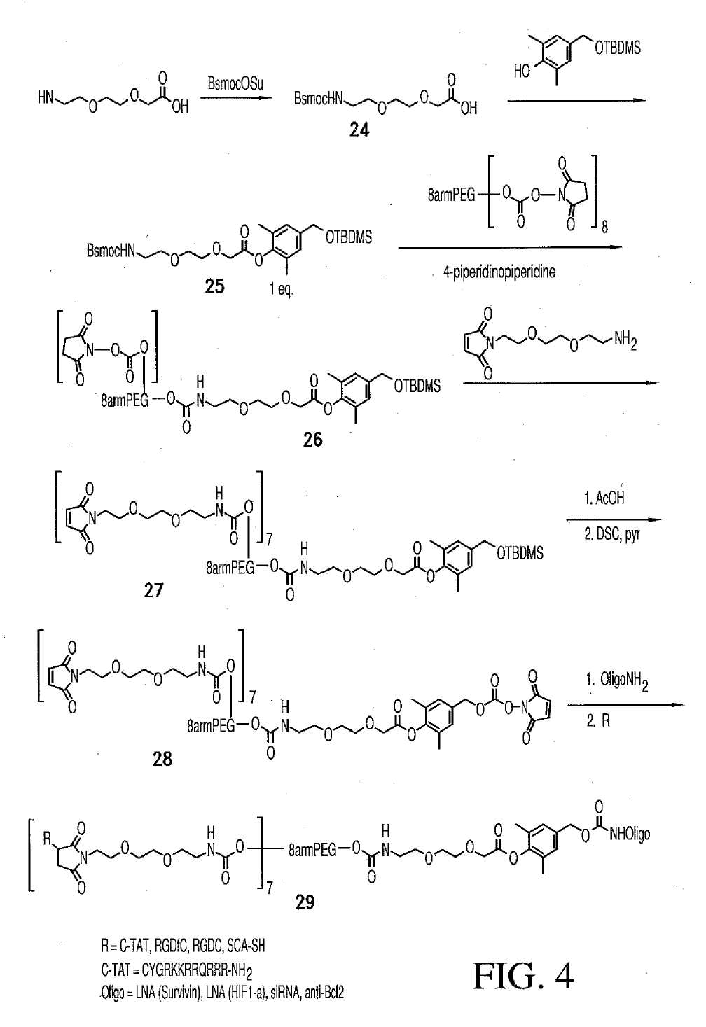

FIG. 5 schematically illustrates methods of synthesis described in Examples 27-31.

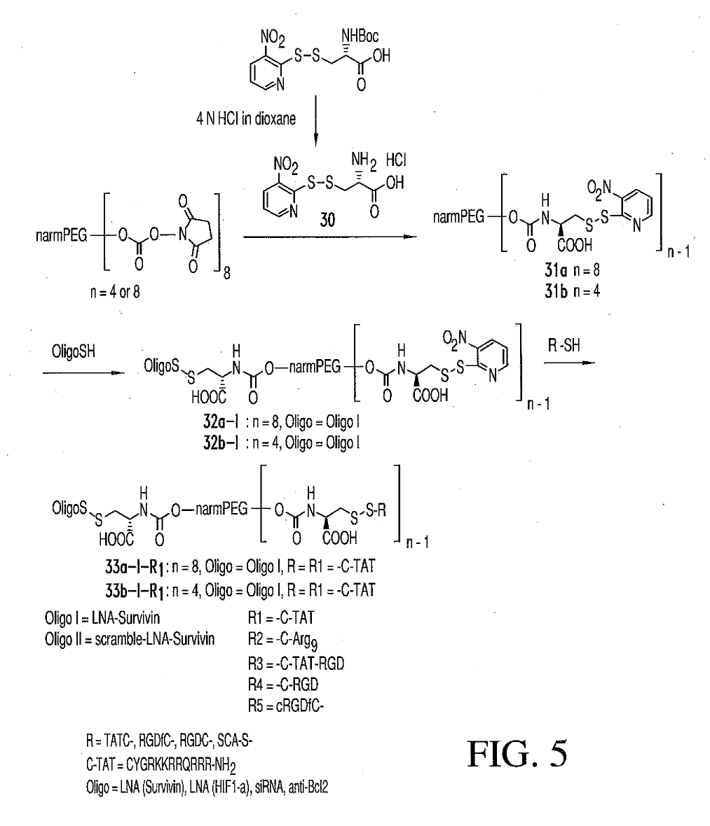

FIG. 6 schematically illustrates methods of synthesis described in Examples 32-34.

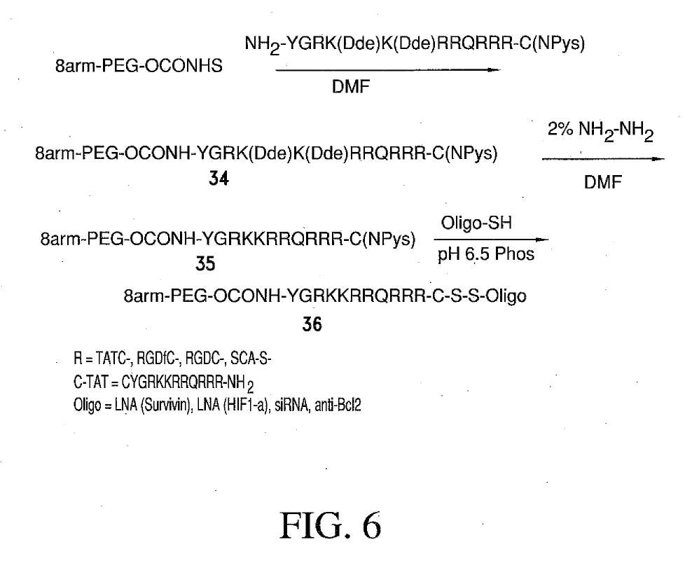

FIG. 7 schematically illustrates methods of synthesis described in Examples 35-38.

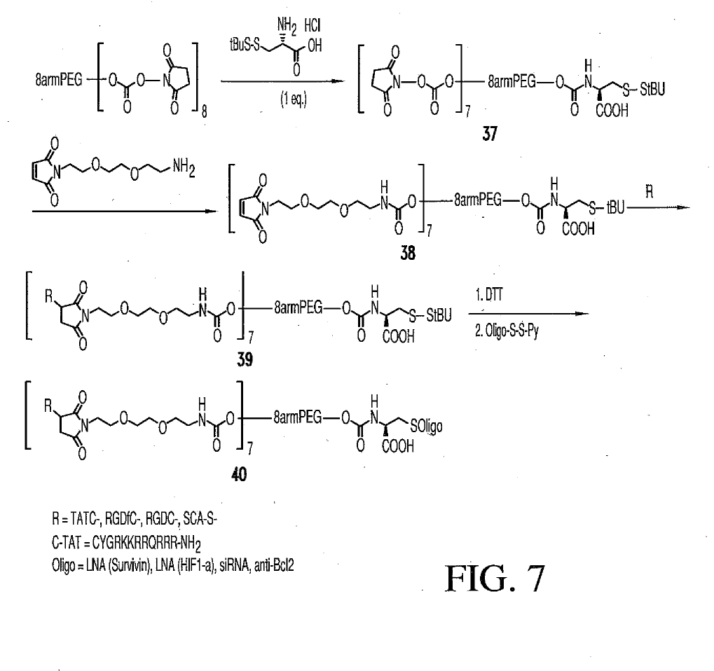

FIG. 8 schematically illustrates methods of synthesis described in Examples 39-41.

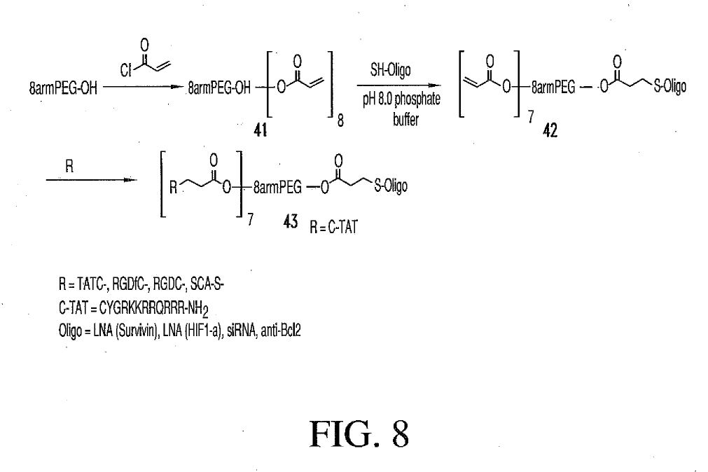

FIG. 9 schematically illustrates methods of synthesis described in Examples 42-49.

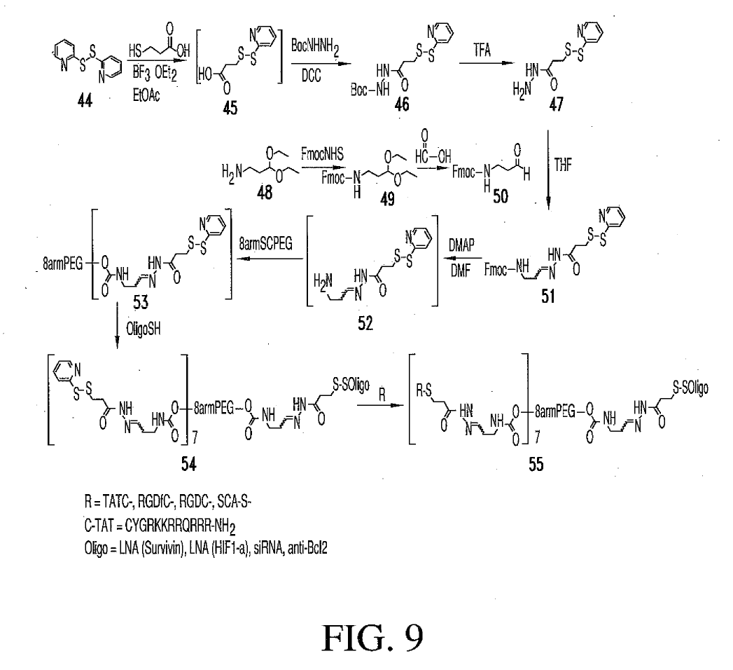

FIG. 10 schematically illustrates methods of synthesis described in Examples 50-53.

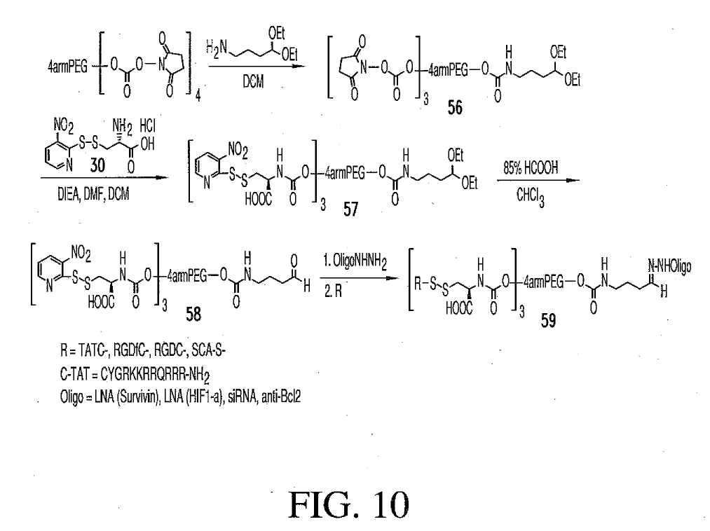

FIG. 11 schematically illustrates methods of synthesis described in Examples 54-58.

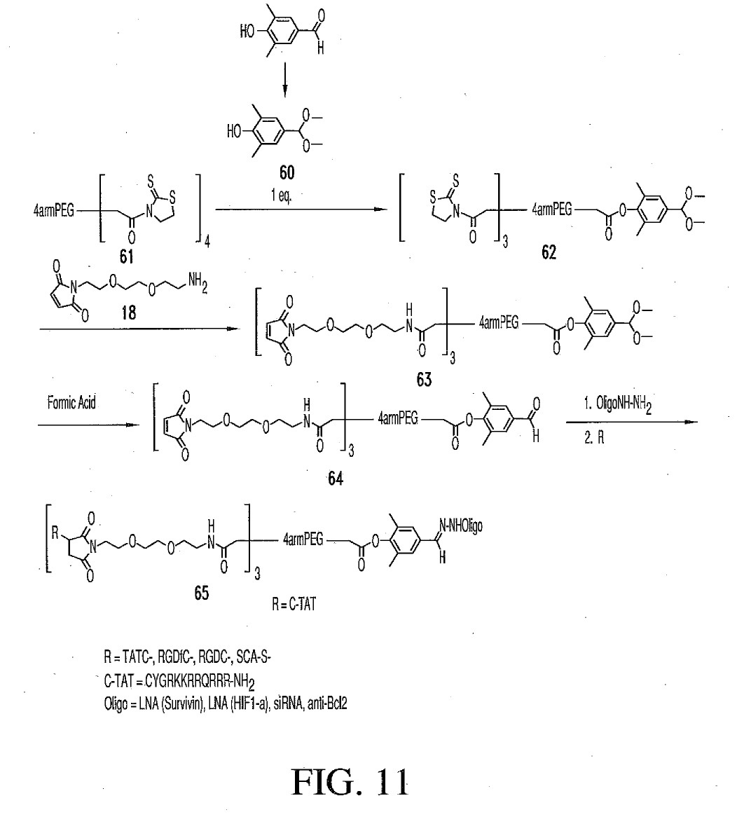

FIG. 12 schematically illustrates methods of synthesis described in Examples 59-62.

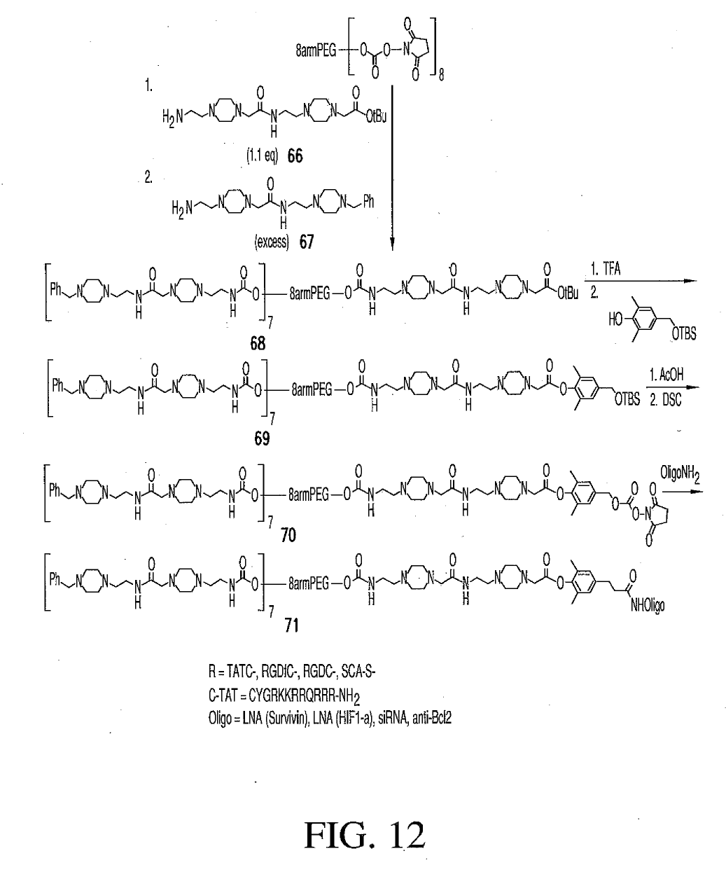

FIG. 13 shows images of fluorescent microscopy described in Example 63.

FIG. 14 shows images of confocal microscopy described in Example 63.

FIG. 15 shows cellular uptakes described in Example 64.

FIG. 16 shows cellular uptakes described in Example 65.

FIG. 17 shows Bcl2 mRNA down regulation described in Example 66.

FIG. 18 shows Survivin downregulation described in Example 67.

FIG. 19 shows Survivin downregulation described in Example 68.

FIG. 20 shows Survivin downregulation described in Example 69.

FIG. 21 shows Survivin downregulation described in Example 70.

FIG. 22 shows Survivin downregulation described in Example 71.

FIG. 23 shows Survivin downregulation described in Example 72.

FIG. 24 shows in vivo Survivin downregulation described in Example 73

### DETAILED DESCRIPTION OF THE INVENTION

### A. Overview

In one aspect of the present invention, there are provided polymeric compounds of formula (I):

{Z₂}_{b}-R₁-{Z₁}ₐ

wherein

each Z₁ is independently

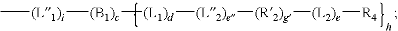

each Z₂ independently selected capping groups,

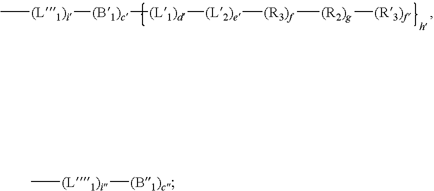

R₁ is a substantially non-antigenic polymer;

R₂ and R′₂ are independently selected positive charge-containing peptides or nitrogen-containing cyclohydrocarbons;

R₃ and R′₃ are independently selected targeting agents;

R₄ is a biologically active moiety;

B₁, B′₁ and B″₁ are independently selected branching groups;

L₁, L′₁, L₁″, L₁′″ and L₁″″ are independently selected bifunctional linkers;

L₂, L′₂ and L″₂ are independently selected releaseable linkers;

(a) is a positive integer, preferably from 1 to about 31, more preferably from about 3 to about 8, and most preferably 1;

(b) is zero or a positive integer, preferably from about 0 to about 31, more preferably from about 3 to about 7;

(c), (c′) and (c″) are independently zero or a positive integer, preferably zero, 1, 2 or 3, and more preferably zero or 1;

(d), (d′), (i), (i′) and (i″) are independently zero or a positive integer, preferably zero, 1, 2 or 3, and more preferably zero or 1;

(e) is a positive integer, preferably 1, 2 or 3, and more preferably 1 or 2;

(e′) and (e″) are independently zero or a positive integer, preferably zero, 1, 2 or 3, and more preferably zero, 1 or 2;

(f) and (f′) are independently zero or a positive integer, preferably zero 1, or 2, and more preferably zero or 1;

(g) is a positive integer, preferably from about 1 to about 5, and more preferably 1 or 2;

(g′) is zero or a positive integer, preferably 0 or an integer from about 1 to about 5, and more preferably zero, 1 or 2; and

(h) and (h′) are independently selected positive integers, preferably from about 1 to about 8, more preferably 1, 2, 3 or 4, and most preferably 1 or 2;

provided that (g′) is a positive integer when (b) is not zero and all Z₂ are capping groups, -(L″″₁)_{i″}-(B″₁)_{c″} or in combination.

For purposes of the present invention, repeating units (a) and (b) adjacent to a bracket can represent the total number of polymer arms bonded to the group described in the bracket with the exception when U-PEG or (PEG)₂-Lys type PEG's are employed as part of the polymeric compounds described herein. The sum of (a) and (b) can be 1 or 3 for U-PEG employed although there are two polymer arms. The polymeric compounds described herein can include mPEG when (a) is 1 and (b) is zero. The polymer terminal of mPEG can be linked to both positively-charged moiety and biologically active material. When bisPEG is employed in the polymeric compounds described herein, the sum of (a) and (b) are 2, in which Z₂ is not a capping group or -(L″″₁)_{i″}-(B″₁)_{c″} when (b) is 1.

In one preferred aspect of the invention, the sum of (a) and (b) equals to from 1 to 32, thus the polymeric compounds can preferably include up to 32 polymer arms, i.e. 1, 2, 3, 4, 8, 16 or 32. Within this embodiment, the polymeric compounds can preferably include from one to eight polymer arms, where the sum of (a) and (b) can be from 1 to 8. More preferably, the polymeric portion includes four polymer arms, where the sum (a) and (b) is 4.

In yet another preferred aspect, the polymeric compounds described herein contain one polymer terminal bonded to a biologically active moiety and each of the remaining polymer terminals bonded to positive charge-containing moieties and targeting agent. Alternatively, more polymer arms of the polymeric portion are linked to positively charged moieties than the biologically active moiety. This feature can confer sufficient positive charges to neutralize the negative charge of the biologically active moiety such as oligonucleotides.

For purposes of the present invention, when the branching group is present within the compounds described herein, any moieties present after the branching moiety to the distal end of each polymer arm are multiplied by the degree of branching, i.e., ×2. (h) and (h)′ represent the number of terminals made according to the branching. In one embodiment, (h) and (h′) can be each 2, where the branching group such as aspartic acid is employed. In other embodiments including one or more branching groups, (h) and (h)′ can be 2, 3, 4, 6, 8, 12, 16, 18, 32 or more. The branching moieties can include at least three functional groups. When a branching moiety having three functional groups such as aspartic acid is linked to the terminal of the polymer arm, each polymer arm can provide functional sites at least twice as many as the number of polymer arms. Multiple branching moieties can be contemplated within the compounds described herein. In another embodiment, (h) and (h′) can be 1 when there is no branching group employed.

In yet another preferred embodiment, four armed polymers can be linked to a branching moiety at each terminal of the polymer arms. The polymeric compounds containing four arms and a branching moiety thereon such as aspartic acid can have 8 functional sites for loading positively-charged moieties and/or a biologically active moiety.

The capping group can be selected from among H, NH₂, OH, CO₂H, C₁₋₆ alkoxy and C₁₋₆ alkyl. Preferably, when a linear polymer such as mPEG is employed in the compounds described herein, the capping group can include methoxy. When (b) is not zero and all Z₂ moieties are capping groups, -(L″″₁)_{i″}-(B″₁)_{c″} or in combination, (g′) is at least 1 so that the positively charged moiety and the biologically active moiety can be employed on the same polymer arm.

In one preferred aspect of the invention, when (b) is not zero, each Z₂ includes

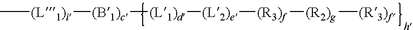

and thus the compounds described herein has the formula (II):

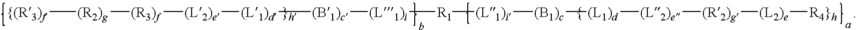

All polymer terminals can be activated and linked to the positively-charged moieties, targeting agents and/or biologically active moieties rather than including a capping group or -(L″″₁)_{i″}-(B″₁)_{c″}. The polymers contemplated with this aspect can therefore include bis-PEGs, U-PEG and multi-arm PEGs.

In another preferred embodiment, (a) is 1. The sum of (a) and (b) can be a positive integer from 1 to 31, preferably 1 to 7, and most preferably 4 (four arm polymers). In yet another preferred aspect, (b) is greater than (a) so that more polymer terminals can have positively-charged moieties than the biologically active moiety to sufficiently neutralize the negative charge of the biologically active moiety such as oligonucleotides.

For purposes of the present invention, when values for bifunctional linkers, branching groups, releasable linkers, positive charge-containing moieties and targeting agents are positive integers equal to or greater than 2, the same or different moieties can be employed. In one embodiment containing two or more releasable linkers, where (e) is equal to or greater than 2, the releasable linkers can be the same or different. In a particular embodiment, a benzyl elimination-based linker is present adjacent to a hydrazone-containing linker in the compounds described herein. In another embodiment, the same or different positively-charged peptides can be employed at the same polymer terminal.

In one preferred embodiment, the compounds described herein have the formula:

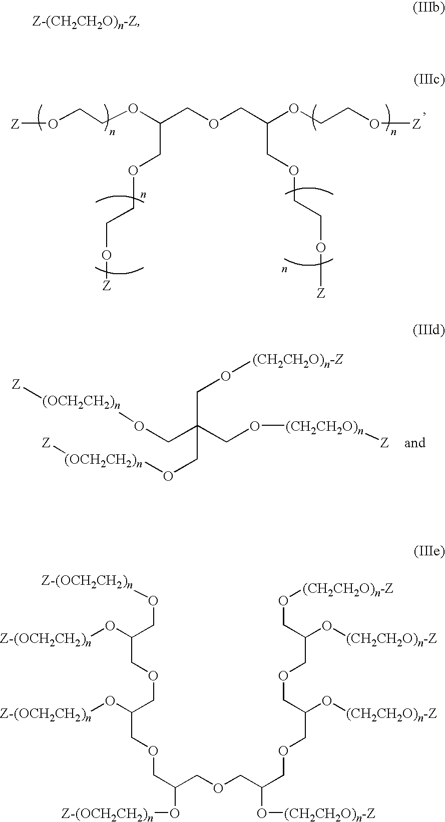

wherein

(n) is an integer from about 10 to about 2300, where the total molecular weight of the polymeric portion is from about 2,000 to about 100,000 daltons;

each Z is Z₁ or Z₂
wherein
each Z₁ is independently.

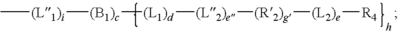

each Z₂ is independently selected capping groups,

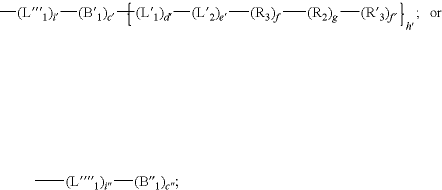

L₂, L′₂ and L″₂ are independently releasable liners selected from among disulfide, hydrazone-containing linkers, thiopropionate-containing linkers, benzyl elimination-based linkers, trialkyl lock-based linkers and bicine-based linkers, lysosomally cleavable peptides and cathepsin B cleavable peptides;
(c), (c′) and (c″) are independently zero or a positive integer, preferably zero, 1, 2 or 3, and more preferably zero or 1;
(d), (d′), (i), (i′) and (i″) are independently zero or a positive integer, preferably zero, 1 or 2;
(e) is a positive integer, preferably 1 or 2;
(e′) and (e″) are independently zero or a positive integer, preferably zero, 1 or 2;
(f′) and (f″) are independently zero or a positive integer, preferably zero, 1 or 2;
(g) is a positive integer, preferably 1 or 2, more preferably 1;
(g′) is zero or a positive integer, preferably zero, 1 or 2;
(h) and (h′) are independently a positive integer, preferably from about 1 to about 8, more preferably 1, 2, 3 or 4, and most preferably 1 or 2; and
all other variables are previously defined,
   provided that (g′) is a positive integer when all Z₂ are capping groups, -(L″″₁)_{i″}-(B″₁)_{c″} or in combination. When the polymeric compounds having four polymer arms, (n) can be from 4 to about 455. The artisans of the ordinary skill can appreciate optional (n) values for other multi-arm polymers. Preferably, all Z₂ moieties are

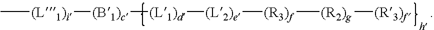

An activated four arm polymer including a branching moiety is illustrated below in Formula (IIIc′)

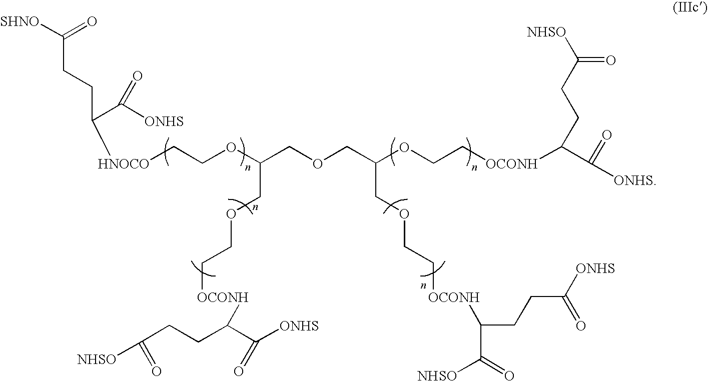

In one preferred aspect of the present invention, the multi-arm polymer conjugates contain one polymer arm terminal attached to a biologically active moiety and each of other polymer arm terminals bonded to a positive charge-containing group.

In further aspect of the present invention, the multi-arm polymer conjugates contain one polymer arm terminal bonded to a biologically active moiety, and each of other polymer arm terminals bonded to a positive charge-containing group and target agent.

### B. Substantially Non-Antigenic Polymers

Polymers employed in the compounds described herein are preferably water soluble polymers and substantially non-antigenic such as polyalkylene oxides (PAO's).

In one aspect of the invention, the compounds described herein include a linear, terminally branched or multi-armed polyalkylene oxide. In some preferred embodiments of the invention, the polyalkylene oxide includes polyethylene glycol and polypropylene glycol.

The polyalkylene oxide has an average molecular weight from about 2,000 to about 100,000 daltons, preferably from about 2,000 to about 60,000 daltons. The polyalkylene oxide can be more preferably from about 5,000 to about 25,000, preferably from about 12,000 to about 20,000 daltons when proteins or oligonucleotides are attached or alternatively from about 20,000 to about 45,000 daltons, preferably from about 30,000 to about 40,000 daltons when pharmaceutically active compounds (small molecules having an average molecular weight of less than 1,500 daltons) are employed in the compounds described herein.

The polyalkylene oxide includes polyethylene glycols and polypropylene glycols. More preferably, the polyalkylene oxide includes polyethylene glycol (PEG). PEG is generally represented by the structure:

—O—(CH₂CH₂O)ₙ—

where (n) is an integer from about 10 to about 2,300, and is dependent on the number of polymer arms when multi-arm polymers are used. Alternatively, the polyethylene glycol (PEG) residue portion of the invention can be represented by the structure:

—Y₇₁—(CH₂CH₂O)ₙ—CH₂CH₂Y₇₁—,

—Y₇₁—(CH₂CH₂O)ₙ—CH₂C(═Y₂₂)—Y₇₁—,

—Y₇₁—C(═Y₇₂)—(CH₂)ₐ₂—Y₇₃—(CH₂CH₂O)ₙ—CH₂CH₂—Y₇₃—(CH₂)ₐ₂—C(═Y₇₂)—Y₇₁— and

—Y₇₁—(CR₇₁R₇₂)ₐ₂—Y₇₃—(CH₂)_{b2}—O—(CH₂CH₂O)ₙ—(CH₂)_{b2}—Y₇₃—(CR₇₁R₇₂)ₐ₂—Y₇₁—,

wherein:

Y₇₁ and Y₇₃ are independently O, S, SO, SO₂, NR₇₃ or a bond;

Y₇₂ is O, S, or NR₇₄;

R₇₁₋₇₄ are independently selected from among hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy and substituted arylcarbonyloxy;

(a2) and (b2) are independently zero or a positive integer, preferably zero or an integer from about 1 to about 6, and more preferably 1; and

(n) is an integer from about 10 to about 2300.

Branched or U-PEG derivatives are described in U.S. Pat. Nos. 5,643,575, 5,919,455, 6,113,906 and 6,566,506, the disclosure of each of which is incorporated herein by reference. A non-limiting list of such polymers corresponds to polymer systems (i)-(vii) with the following structures:

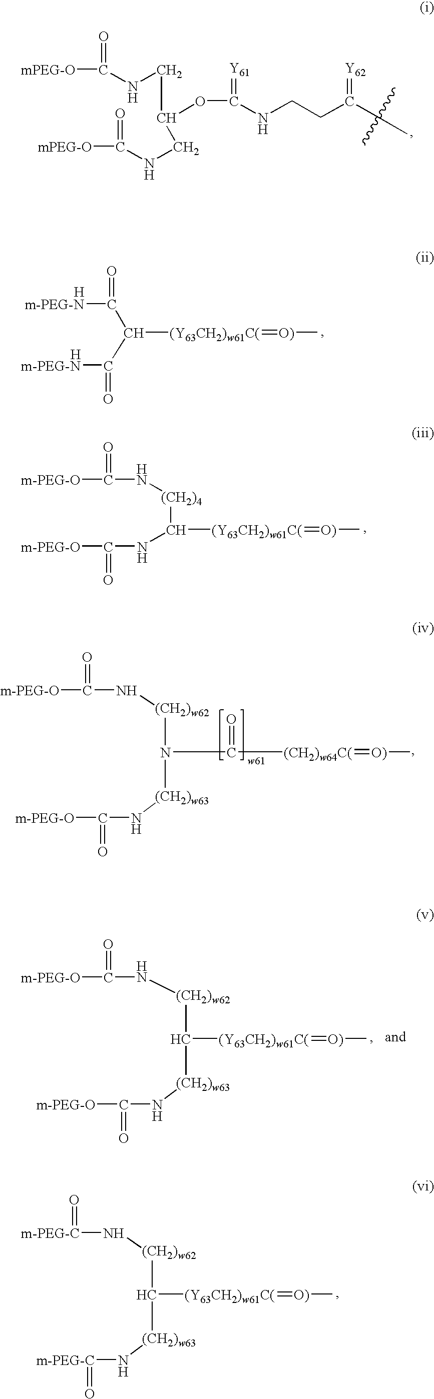

wherein:

Y₆₁₋₆₂ are independently O, S or NR₆₁;

Y₆₃ is O, NR₆₂, S, SO or SO₂

(w62), (w63) and (w64) are independently 0 or a positive integer, preferably zero or an integer from about 1 to about 3;

(w61) is 0 or 1;

mPEG is methoxy PEG
wherein PEG is previously defined and a total molecular weight of the polymer portion is from about 2,000 to about 100,000 daltons; and

R₆₁ and R₆₂ are independently the same moieties which can be used for R₇₃.

In yet another aspect, the polymers include multi-arm PEG-OH or “star-PEG” products such as those described in NOF Corp. Drug Delivery System catalog, Ver. 8, April 2006, the disclosure of which is incorporated herein by reference. The multi-arm polymer conjugates contain four or more polymer arms and preferably four or eight polymer arms.

For purposes of illustration and not limitation, the multi-arm polyethylene glycol (PEG) residue can be

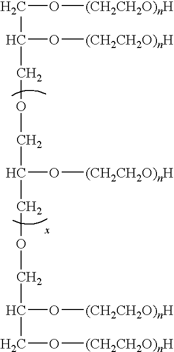

wherein:

(x) is zero and a positive integer, i.e. from about 0 to about 28; and

(n) is the degree of polymerization.

In one particular embodiment of the present invention, the multi-arm PEG has the structure:

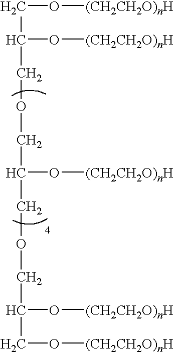

wherein (n) is a positive integer. In one preferred embodiment of the invention, the polymers have a total molecular weight of from about 5,000 Da to about 60,000 Da, and preferably from 12,000 Da to 40,000 Da.

In yet another particular embodiment, the multi-arm PEG has the structure:

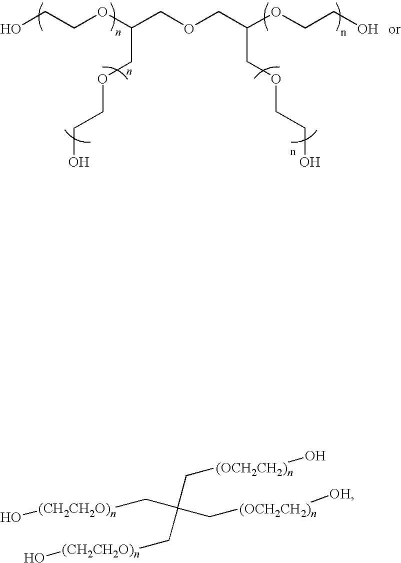

Wherein (n) is a positive integer. In one preferred embodiment of the invention, the degree of polymerization for the multi-arm polymer (n) is from about 28 to about 350 to provide polymers having a total molecular weight of from about 5,000 Da to about 60,000 Da, and preferably from about 65 to about 270 to provide polymers having a total molecular weight of from 12,000 Da to 45,000 Da. This represents the number of repeating units in the polymer chain and is dependent on the molecular weight of the polymer.

The polymers can be converted into a suitably activated polymer, using the activation techniques described in U.S. Pat. No. 5,122,614 or 5,808,096. Specifically, such PEG can be of the formula:

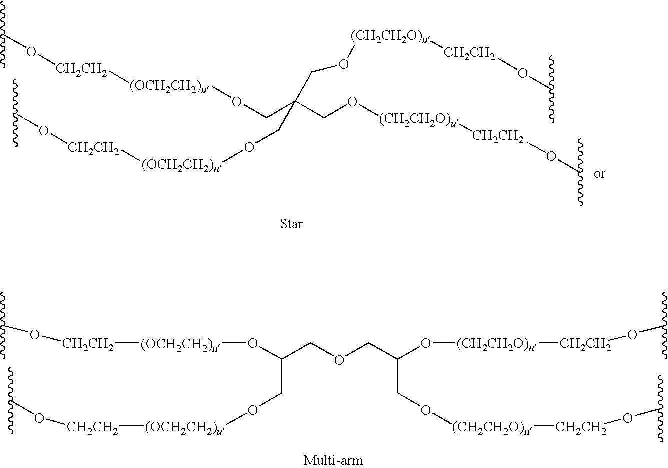

wherein:

(u′) is an integer from about 4 to about 455; and up to 3 terminal portions of the residue is/are capped with a methyl or other lower alkyl.

In some preferred embodiments, all four of the PEG arms can be converted to suitable activating groups, for facilitating attachment to aromatic groups. Such compounds prior to conversion include:

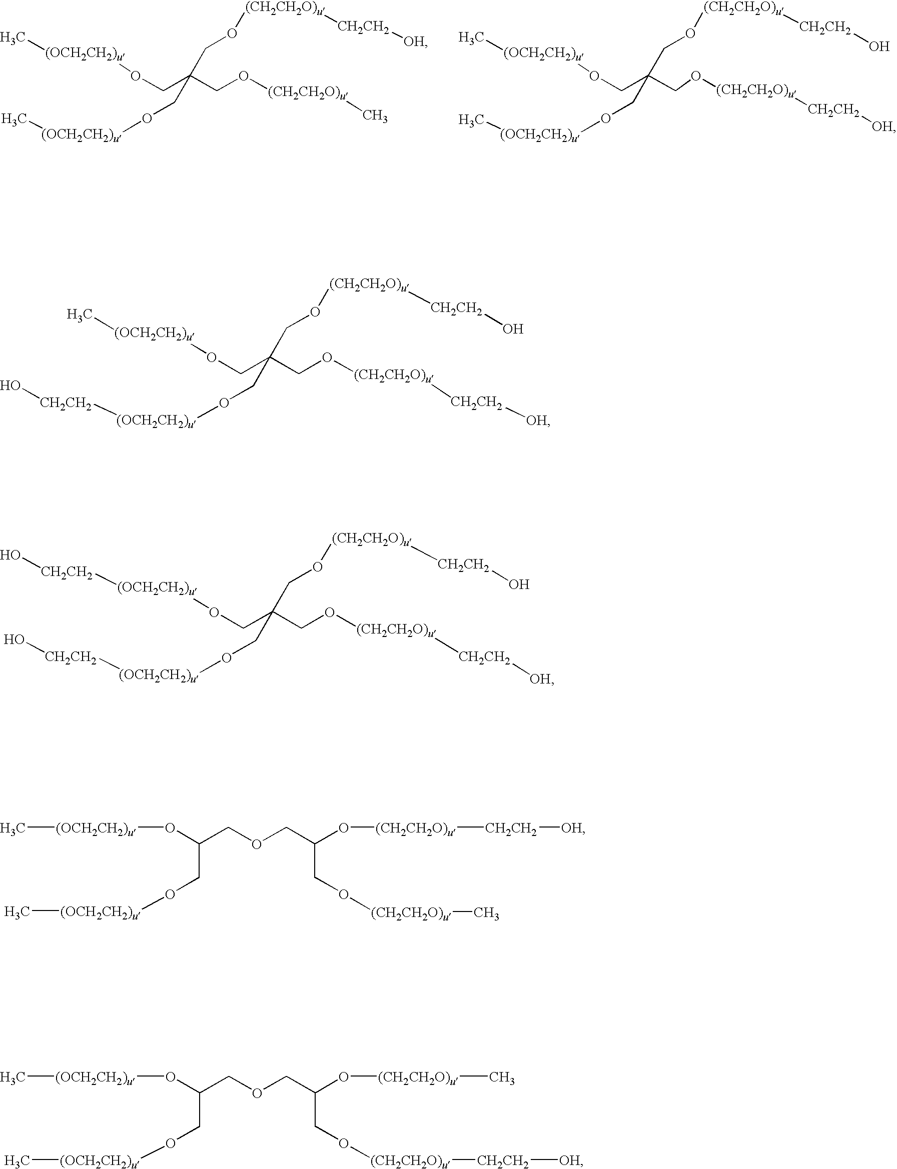

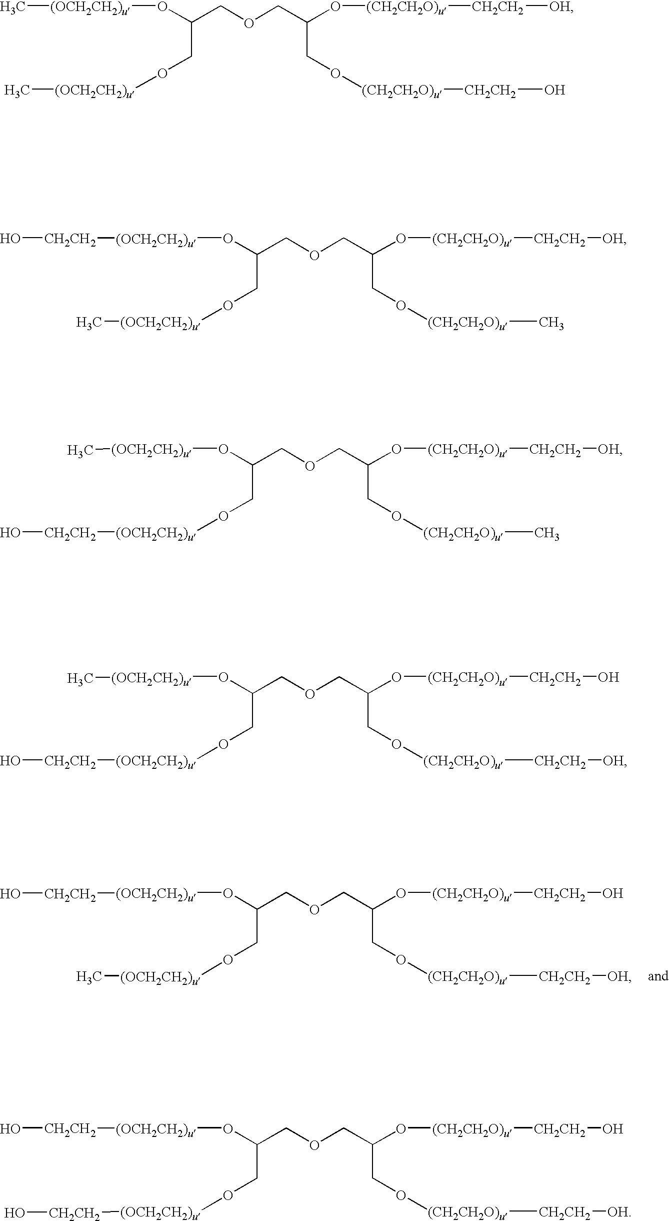

The polymeric substances included herein are preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

In a further embodiment, and as an alternative to PAO-based polymers, one or more effectively non-antigenic materials such as dextran, polyvinyl alcohols, carbohydrate-based polymers, hydroxypropylmethacrylamide (HPMA), polyalkylene oxides, and/or copolymers thereof can be used. See also commonly-assigned U.S. Pat. No. 6,153,655, the contents of which are incorporated herein by reference. It will be understood by those of ordinary skill that the same type of activation is employed as described herein as for PAO's such as PEG. Those of ordinary skill in the art will further realize that the foregoing list is merely illustrative and that all polymeric materials having the qualities described herein are contemplated. For purposes of the present invention, “substantially or effectively non-antigenic” means all materials understood in the art as being nontoxic and not eliciting an appreciable immunogenic response in mammals.

In some aspects, polymers having terminal amine groups can be employed to make the compounds described herein. The methods of preparing polymers containing terminal amines in high purity are described in U.S. patent application Ser. Nos. 11/508,507 and 11/537,172, the contents of each of which are incorporated by reference. For example, polymers having azides react with phosphine-based reducing agent such as triphenylphosphine or an alkali metal borohydride reducing agent such as NaBH₄. Alternatively, polymers including leaving groups react with protected amine salts such as potassium salt of methyl-tert-butyl imidodicarbonate (KNMeBoc) or the potassium salt of di-tert-butyl imidodicarbonate (KNBoc₂) followed by deprotecting the protected amine group. The purity of the polymers containing the terminal amines formed by these processes is greater than about 95% and preferably greater than 99%.

In alternative aspects, polymers having terminal carboxylic acid groups can be employed in the polymeric delivery systems described herein. Methods of preparing polymers having terminal carboxylic acids in high purity are described in U.S. patent application Ser. No. 11/328,662, the contents of which are incorporated herein by reference. The methods include first preparing a tertiary alkyl ester of a polyalkylene oxide followed by conversion to the carboxylic acid derivative thereof. The first step of the preparation of the PAO carboxylic acids of the process includes forming an intermediate such as t-butyl ester of polyalkylene oxide carboxylic acid. This intermediate is formed by reacting a PAO with a t-butyl haloacetate in the presence of a base such as potassium t-butoxide. Once the t-butyl ester intermediate has been formed, the carboxylic acid derivative of the polyalkylene oxide can be readily provided in purities exceeding 92%, preferably exceeding 97%, more preferably exceeding 99% and most preferably exceeding 99.5% purity.

### C. Positive Charge Containing Moieties

The polymeric compounds described herein can contain positively-charged peptides or nitrogen-containing cyclohydrocarbons. The positive charge-containing moieties are capable of conferring additional positive charges to the substantially non-antigenic polymer.

The positively charged peptides can help the polymeric compounds penetrate cell membrane. Cell penetrating peptides (CPPs) contain positively-charged amino acids such as arginine, and lysine. CPPs also facilitate targeted delivery of the polymeric compounds described herein.

In one aspect of the present invention, one or more peptides can be employed in the compounds described herein. The positively charged peptides can be employed in the compounds in a number of different combinations. For purposes of illustration and not limitation, optional combination is provided. In one embodiment, multiple units of the peptides such as two TAT sequences can be attached in a row.

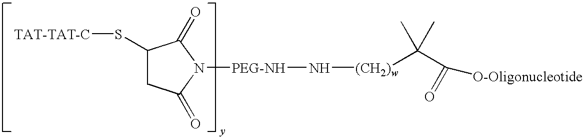

wherein (w) is a positive integer from about 1 to about 10, preferably from about 3 to about 7; and (y) is an integer from about 1 to about 7.

In another embodiment, each of two or more peptides can be linked to each of the polymer arm terminal via a branching group to enhance cellular uptake.

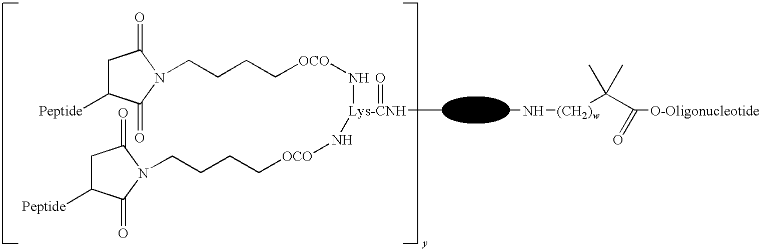

wherein (w) is an integer from about 1 to about 10 and (y) is an integer from about 1 to about 7.

The peptides can contains from about 1 to about 50 positively charged amino acids, preferably from about 2 to about 20, and more preferably 3 to 10.

In one preferred embodiment, the positively-charged peptides include cell penetrating peptides (CPPs) such as TAT, Penetratin and (Arg)₉. See Curr Opin Pharmacol. 2006 October; 6(5):509-14, Cell-penetrating peptides as vectors for peptide, protein and oligonucleotide delivery, the contents of which are incorporated herein by reference.

In one aspect, the positively-charged peptides can include naturally occurring amino acids or non-naturally occurring amino acids. Preferably, the peptides include arginine, lysine and related analogs. The peptides can be random sequences of amino acids or part of naturally occurring cell penetrating peptides or their derivatives.

For purposes of the present invention, the peptides contemplated in the polymeric compounds described herein can include cysteine at the end of the peptides or within the peptides for further conjugating or introducing disulfide bond.

One preferred embodiment of the present invention includes positively-charged peptide of trans-activator of transcription protein (TAT). For purposes of the present invention, the term “TAT” can be understood to mean a portion of trans-activator of transcription activation protein including a peptide sequence of YGRKKRRQRRR, for example, HS—CYGRKKRRQRRR—CONH₂.

C-TAT: CYGRKKRRQRRR (SEQ ID NO: 1)

In another preferred embodiment, the positively-charged peptide can be polyarginine such as (Arg)₅, or NH(Me)-Sar-Arg-Arg-Arg-Arg-Arg-CONH₂ (“Sar-(Arg)₅”).

C-(Arg)₉: CRRRRRRRRR (SEQ ID NO: 2)

Other peptide groups suitable for inclusion herein will be apparent to those of ordinary skill provided that they include a sufficient number of positive charged-groups. The length of the peptide will also vary according to the needs of the artisan and the number of positive charge groups (provided by the amino acids) desired.

In some preferred embodiments, the peptides will contain from about 1 to about 50, preferably from about 2 to about 20 and more preferably from about 3 to about 10 positively charged amino acids therein. See also Zhao, H., et al, Bioconjugate Chem., 2005, 16: 758-766, the contents of which are incorporated by reference herein.

When the positively charged peptides are attached to a targeting moiety such as SCA, a linker can be inserted for conjugating SCA to the positively charged peptides. The linkers known to those of ordinary skill are also contemplated as being within the compounds described herein.

In an alternative aspect, the positive charge containing moieties includes nitrogen-containing cyclohydrocarbons. The nitrogen-containing moieties correspond to the formula:

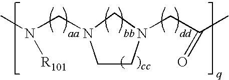

wherein

(aa) is a positive integer from about 2 to about 10, preferably 2 or 3, and more preferably 2;

(bb) is 1, 2 or 3;

(cc) is 1 or 2;

(dd) is a positive integer from about 1 to about 5, preferably 1;

R₁₀₁ is independently selected from among hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, substituted and arylcarbonyloxy, and

(q) is an positive integer from about 2 to about 30.

In one preferred embodiment, (q) is from about 3 to about 18 and thus, each terminal of the polymer arms contains 3 up to 18 cyclohydrocarbon units. More preferably, (q) is from about 3 to 9.

In one preferred embodiment, the nitrogen-containing cyclohydrocarbon can be selected from among:

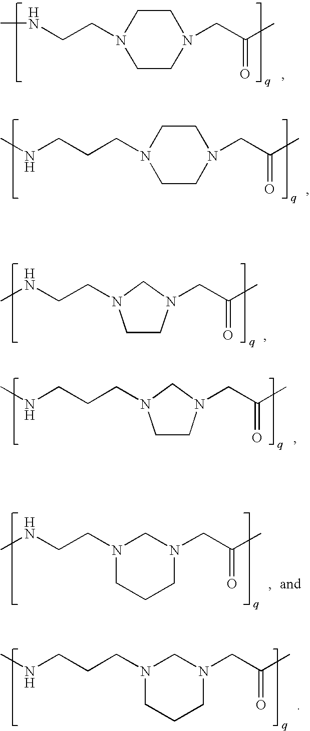

The nitrogen-containing cyclohydrocarbon moiety preferably contains piperazine.

### D. Biologically Active Moieties

The compounds described herein can be used for delivering various negatively-charged molecules. The polymer compounds improve the cellular uptake as well as biodistribution of negatively charged molecules. The negatively charged molecules can include pharmaceutically active compounds (small molecular weight compounds having an average molecular weight of less than 1,500 daltons), enzymes, proteins, oligonucleotides, antibodies, monoclonal antibodies, single chain antibodies and peptides. The biologically active moieties can be —NH₂ containing moieties, —OH containing moieties and —SH containing moieties.

In one preferred embodiment, the biologically active moieties include an oligonucleotide.

In order to more fully appreciate the scope of the present invention, the following terms are defined. The artisan will appreciate that the terms, “nucleic acid” or “nucleotide” apply to deoxyribonucleic acid (“DNA”), ribonucleic acid, (“RNA) whether single-stranded or double-stranded, unless otherwise specified, and any chemical modifications thereof. An “oligonucleotide” is generally a relatively short polynucleotide, e.g., ranging in size from about 2 to about 200 nucleotides, or more preferably from about 10 to about 30 nucleotides in length. The oligonucleotides according to the invention are generally synthetic nucleic acids, and are single stranded, unless otherwise specified. The terms, “polynucleotide” and “polynucleic acid” may also be used synonymously herein.

The term “antisense,” as used herein, refers to nucleotide sequences which are complementary to a specific DNA or RNA sequence that encodes a gene product or that encodes a control sequence. The term “antisense strand” is used in reference to a nucleic acid strand that is complementary to the “sense” strand. In the normal operation of cellular metabolism, the sense strand of a DNA molecule is the strand that encodes polypeptides and/or other gene products. The sense strand serves as a template for synthesis of a messenger RNA (“mRNA”) transcript (an antisense strand) which, in turn, directs synthesis of any encoded gene product. Antisense nucleic acid molecules may be produced by any art-known methods, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation. In this manner, mutant phenotypes may be generated. The designations “negative” or (−) are also art-known to refer to the antisense strand, and “positive” or (+) are also art-known to refer to the sense strand

In one preferred embodiment, the choice for conjugation is an oligonucleotide (or “polynucleotide”) and after conjugation, the target is referred to as a residue of an oligonucleotide. The oligonucleotides can be selected from among any of the known oligonucleotides and oligodeoxynucleotides with phosphorodiester backbones or phosphorothioate backbones.

The oligonucleotides (analogs) are not limited to a single species of oligonucleotide but, instead, are designed to work with a wide variety of such moieties, it being understood that linkers can attach to one or more of the 3′- or 5′-terminals, usually PO₄ or SO₄ groups of a nucleotide. The oligonucleotides include antisense oligonucleotides, short interfering RNA (siRNA), micro RNA (miRNA), aptamer, etc. The oligonucleotides or oligonucletide derivatives can include from about 10 to about 1000 nucleic acids, and preferably relatively short polynucleotides, e.g., ranging in size from about 2 to about 200 nucleotides, or more preferably from about 10 to about 30 nucleotides in length. In addition, the oligonucleotides can contain natural phosphorodiester backbone or phosphorothioate backbone or any other modified backbone analogues such as LNA (Locked Nucleic Acid), PNA (nucleic acid with peptide backbone), tricyclo-DNA; decoy ODN (double stranded oligonucleotide), RNA (catalytic-RNA sequence), ribozymes; spiegelmers (L-conformational oligonucleotides), CpG oligomers, and the like, such as those disclosed at Tides 2002, Oligonucleotide and Peptide Technology Conferences, May 6-8, 2002, Las Vegas, Nev. and Oligonucleotide & Peptide Technologies, 18th & 19 Nov. 2003, Hamburg, Germany, the contents of which are incorporated herein by reference.

Oligonucleotides according to the invention can also optionally include any suitable art-known nucleotide analogs and derivatives, including those listed by Table 1, below.

TABLE 1
Representative Nucleotide Analogs And Derivatives

| | |
|---|---|
| 4-acetylcytidine | 5-methoxyaminomethyl-2-thiouridine |
| 5-(carboxyhydroxymethyl) | beta, D-mannosylqueuosine |
| uridine | |
| 2′-O-methylcytidine | 5-methoxycarbonylmethyl-2-thiouridine |
| 5-carboxymethylaminomethyl-2- | 5-methoxycarbonylmethyluridine |
| thiouridine | |
| 5-carboxymethylaminomethyl- | 5-methoxyuridine |
| uridine | |
| Dihydrouridine | 2-methylthio-N6-isopentenyladenosine |
| 2′-O-methylpseudouridine | N-((9-beta-D-ribofuranosyl-2- |
| | methylthiopurine-6- |
| | yl)carbamoyl)threonine |
| D-galactosylqueuosine | N-((9-beta-D-ribofuranosylpurine-6- |
| | yl)N-methylcarbamoyl)threonine |
| 2′-O-methylguanosine | uridine-5-oxyacetic acid-methylester |
| Inosine | uridine-5-oxyacetic acid |
| N6-isopentenyladenosine | Wybutoxosine |
| 1-methyladenosine | Pseudouridine |
| 1-methylpseudouridine | Queuosine |
| 1-methylguanosine | 2-thiocytidine |
| 1-methylinosine | 5-methyl-2-thiouridine |
| 2,2-dimethylguanosine | 2-thiouridine |
| 2-methyladenosine | 4-thiouridine |
| 2-methylguanosine | 5-methyluridine |
| 3-methylcytidine | N-((9-beta-D-ribofuranosylpurine-6-yl)- |
| | carbamoyl)threonine |
| 5-methylcytidine | 2′-O-methyl-5-methyluridine |
| N6-methyladenosine | 2′-O-methyluridine |
| 7-methylguanosine | Wybutosine |
| 5-methylaminomethyluridine | 3-(3-amino-3-carboxy-propyl)uridine |
| Locked-adenosine | Locked-cytidine |
| Locked-guanosine | Locked-thymine |
| Locked-uridine | Locked-methylcytidine |

Modifications to the oligonucleotides contemplated in the invention include, for example, the addition to or substitution of selected nucleotides with functional groups or moieties that permit covalent linkage of an oligonucleotide to a desirable polymer, and/or the addition or substitution of functional moieties that incorporate additional charge, polarizability, hydrogen bonding, electrostatic interaction, and functionality to an oligonucleotide. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodouracil, backbone modifications, methylations, base-pairing combinations such as the isobases isocytidine and isoguanidine, and analogous combinations. Oligonucleotide modifications can also include 3′ and 5′ modifications such as capping. Structures of illustrative nucleoside analogs are provided below.

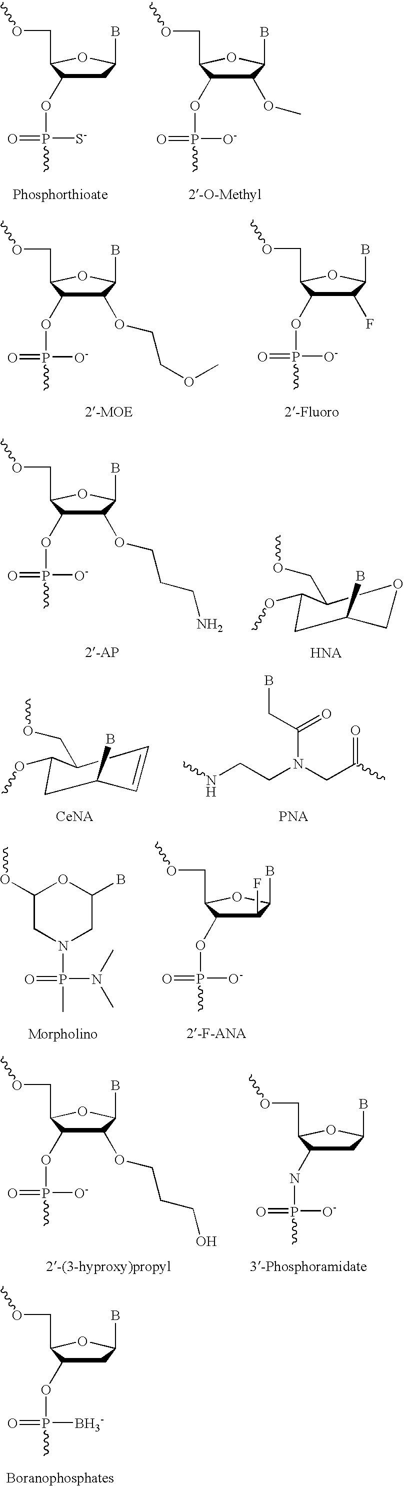

See more examples of nucleoside analogues described in Freier & Altmann; Nucl. Acid Res., 1997, 25, 4429-4443 and Uhlmann; Curr. Opinion in Drug Development 2000, 3(2), 293-213, the contents of each of which are incorporated herein by reference.

In one preferred aspect of the present invention, the oligonucleotide is involved in targeted tumor cells or downregulating a protein implicated in the resistance of tumor cells to anticancer therapeutics. For example, any art-known cellular proteins such as bcl-2 for downregulation by antisense oligonucleotides, for cancer therapy, can be used for the present invention. See U.S. patent application Ser. No. 10/822,205 filed Apr. 9, 2004, the contents of which are incorporated by reference herein. A non-limiting list of preferred therapeutic oligonucleotides include antisense HIF-1α oligonucleotides and antisense Survivin oligonucleotides.

The oligonucleotide can be, for example, an oligonucleotide that has the same or substantially similar nucleotide sequence as does Genasense (a/k/a oblimersen sodium, produced by Genta Inc., Berkeley Heights, N.J.). Genasense is an 18-mer phosphorothioate antisense oligonucleotide, TCTCCCAGCGTGCGCCAT (SEQ ID NO: 6), that is complementary to the first six codons of the initiating sequence of the human bcl-2 mRNA (human bcl-2 mRNA is art-known, and is described, e.g., as SEQ ID NO: 19 in U.S. Pat. No. 6,414,134, incorporated by reference herein). The U.S. Food and Drug Administration (FDA) gave Genasense Orphan Drug status in August 2000. Preferred embodiments include:

(i) antisense Survivin LNA (SEQ ID NO: 3)

^{m}Cₛ-Tₛ-^{m}Cₛ-Aₛ-aₛ-tₛ-cₛ-C_{S}-aₛ-tₛ-gₛ-gₛ-^{m}Cₛ-Aₛ-Gₛ-c;

where the upper case letter represents LNA, the “s” represents a phosphorothioate backbone;

(ii) antisense Bcl2 siRNA:

| | |
|---|---|
| SENSE | |
| 5′- GCAUGCGGCCUCUGUUUGAdTdT-3′ | (SEQ ID NO: 4) |
| ANTISENSE | |
| 3′- dTdTCGUACGCCGGAGACAAACU-5′ | (SEQ ID NO: 5) |

where dT represents DNA;

(iii) Genasense (phosphorothioate antisense oligonucleotide): (SEQ ID NO: 6)

tₛ-cₛ-tₛ-cₛ-cₛ-cₛ-aₛ-gₛ-cₛ-gₛ-tₛ-gₛ-cₛ-gₛ-cₛ-cₛ-
cₛ-aₛ-t

where the lower case letter represents DNA and “s” represents phosphorothioate backbone;

(iv) antisense HIF1α LNA (SEQ ID: 7)

(SEQ ID NO: 7)

5′-ₛTₛGₛGₛcₛaₛaₛgₛcₛaₛtₛcₛcₛTₛGₛTₛa-3′

where the upper case letter represents LNA and the “s” represents phosphorothioate backbone.

LNA includes 2′-O, 4′-C methylene bicyclonucleotide as shown below:

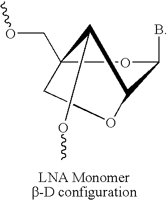

See Detailed description of Survivin LNA disclosed in U.S. patent application Ser. Nos. 11/272,124, entitled “LNA Oligonucleotides and the Treatment of Cancer” and 10/776,934, entitled “Oligomeric Compounds for the Modulation Survivin Expression”, the contents of each of which are incorporated herein by reference. See also U.S. patent application Ser. Nos. 10/407,807, entitled “Oligomeric Compounds for the Modulation HIF-1 Alpha Expression” and 11/271,686, entitled “Potent LNA Oligonucleotides for Inhibition of HIF-1A Expression”, the contents of which are also incorporated herein by reference.

The oligonucleotides employed in the compounds described herein can be modified with (CH₂)_{w} amino linkers at 5′ or 3′ end of the oligonucleotides, where (w) in this aspect is a positive integer of preferably from about 1 to about 10, preferably 6. The modified oligonucleotides can be NH—(CH₂)_{w}-Oligonucleotide as shown below

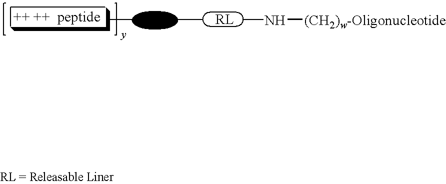

wherein (y) is an integer from about 1 to about 7.

In one preferred embodiment, 5′ end of the sense strand of siRNA is modified. For example, siRNA employed in the polymeric conjugates is modified with a 5′-C₆—NH₂. One particular embodiment of the present invention employs Bcl2-siRNA having the sequence of

SENSE 5-(NH₂—C₆)GCAUGCGGCCUCUGUUUGAdTdT-3′

ANTISENSE 3′-dTdTCGUACGCCGGAGACAAACU-5′.

In alternative aspect, the compounds described herein can include oligonucleotides modified with hindered ester-containing (CH₂)_{w} amino linkers. See U.S. Provisional Application Nos. 60/844,942 entitled “Polyalkylene Oxides Having Hindered Ester-Based Biodegradable Linkers” and 60/845,028 entitled “Hindered Ester-Based Biodegradable Linkers for Oligonucleotide Delivery”, the contents of each of which are incorporated by reference. The polymeric compounds can release the oligonucleotides without amino tail. For example, the oligonucleotides can have the structure:

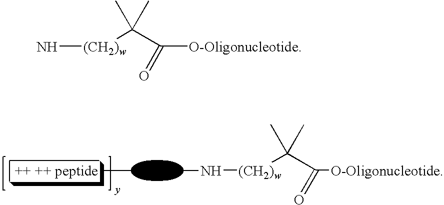

In yet alternative aspect, oligonucleotides can be modified with (CH₂) sulfhydryl linkers (thio oligonucleotides). The thio oligonucleotides can be used for conjugating directly to cysteine of the positively charge peptide or via maleimidyl group. The thio oligonucleotides can have the structure SH—(CH₂)_{w}-Oligonucleotide. The thio oligonucleotides can also include hindered ester having the structure:

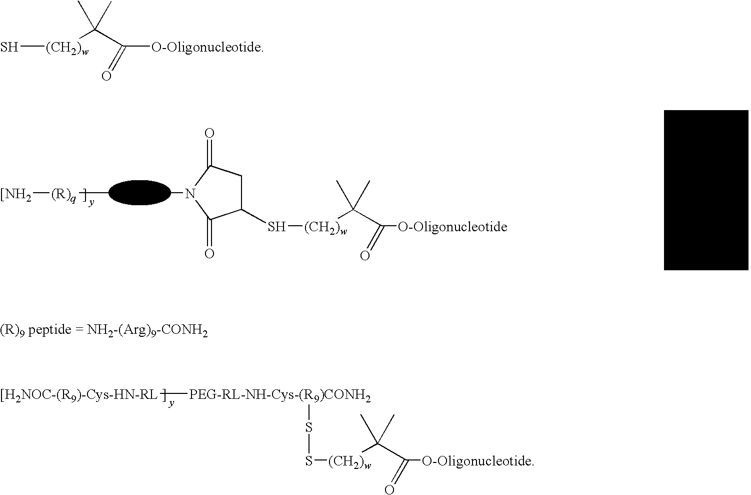

The oligonucleotides can be modified with a C₆—NH₂ tail, a C₆—SH tail or a hindered ester tail. Exemplary of the modified oligonucleotides include:

(i) Genasense modified with a C₆—NH₂ tail:

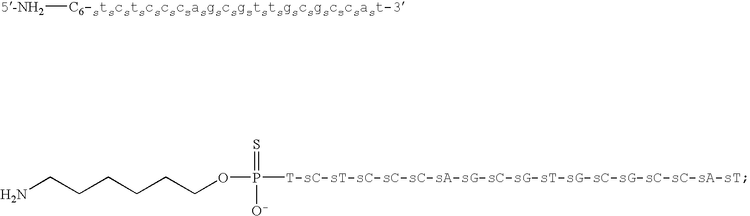

(iii) antisense HIF1a LNA modified with a C₆—NH₂ tail:

(iv) 5′-NH₂—C₆-ₛTₛGₛGₛcₛaₛaₛgₛcₛaₛtₛcₛcₛTₛGₛTₛa-3′;

(iii) antisense Survivin LNA modified with a C₆—NH₂ tail:
5′-NH₂—C₆-ₛ^{m}CₛTₛ^{m}CₛAₛaₛtₛcₛcₛaₛtₛgₛ^{m}CₛAₛGₛc-3′;

(iv) antisense Survivin LNA modified with a C₆—SH tail
5′-HS—C₆-ₛ^{m}CₛTₛ^{m}CₛAₛaₛtₛcₛcₛaₛtₛgₛgₛ^{m}CₛAₛGₛc-3′;

(v) Genasense modified with a hindered ester tail

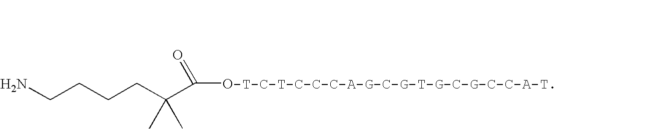

### E. Targeting Agents

Targeting agents can be attached to the polymeric compounds described herein to guide the conjugates to the target area in vivo. The targeting agents allow negatively charged biologically active moieties such as oligonucleotides to have therapeutic efficacies at the target area, i.e. tumor site. The targeted delivery of negatively-charged molecules such as oligonucleotides in vivo enhances the cellular uptake of these molecules to have better therapeutic efficacies. In certain aspects, some cell penetrating peptides can be replaced with a variety of targeting peptides for targeted delivery to the tumor site.

In one preferred aspect of the invention, the targeting moiety, such as a single chain antibody (SCA) or single-chain antigen-binding antibody, monoclonal antibody, cell adhesion peptides such as RGD peptides and Selectin, cell penetrating peptides (CPPs) such as TAT, Penetratin and (Arg)₉, receptor ligands, targeting carbohydrate molecules or lectins, oligonucleotide, oligonucleotide derivatives such as locked nucleic acid (LNA) and aptamers, or the like, allows cytotoxic drugs to be specifically directed to targeted regions. See J Pharm Sci. 2006 September; 95(9):1856-72 Cell adhesion molecules for targeted drug delivery, the contents of which are incorporated herein by reference.

Preferred targeting moieties include single-chain antibodies (SCA's) or single-chain variable fragments of antibodies (sFv). The SCA contains domains of antibodies which can bind or recognize specific molecules of targeting tumor cells. In addition to maintaining an antigen binding site, a PEGylated SCA through linkers can reduce antigenicity and increase the half life of the SCA in the bloodstream.

The terms “single chain antibody” (SCA), “single-chain antigen-binding molecule or antibody” or “single-chain Fv” (sFv) are used interchangeably. The single chain antibody has binding affinity for the antigen. Single chain antibody (SCA) or single-chain Fvs can and have been constructed in several ways. A description of the theory and production of single-chain antigen-binding proteins is found in commonly assigned U.S. patent application Ser. No. 10/915,069 and U.S. Pat. No. 6,824,782, the contents of each of which are incorporated by reference herein.

Typically, SCA or Fv domains can be selected among monoclonal antibodies known by their abbreviations in the literature as 26-10, MOPC 315, 741F8, 520C9, McPC 603, D1.3, murine phOx, human phOx, RFL3.8 sTCR, 1A6, Se155-4,18-2-3,4-4-20,7A4-1, B6.2, CC49,3C2,2c, MA-15C5/K₁₂ G_{O}, Ox, etc. (see, Huston, J. S. et al., Proc. Natl. Acad. Sci. USA 85:5879-5883 (1988); Huston, J. S. et al., SIM News 38(4) (Supp):11 (1988); McCartney, J. et al., ICSU Short Reports 10:114 (1990); McCartney, J. E. et al., unpublished results (1990); Nedelman, M. A. et al., J. Nuclear Med. 32 (Supp.):1005 (1991); Huston, J. et al., In: Molecular Design and Modeling: Concepts and Applications, Part B, edited by J. J. Langone, Methods in Enzymology 203:46-88 (1991); Huston, J. S. et al., In: Advances in the Applications of Monoclonal Antibodies in Clinical Oncology, Epenetos, A. A. (Ed.), London, Chapman & Hall (1993); Bird, R. E. et al., Science 242:423-426 (1988); Bedzyk, W. D. et al., J. Biol. Chem. 265:18615-18620 (1990); Colcher, D. et al., J. Nat. Cancer. Inst. 82:1191-1197 (1990); Gibbs, R. A. et al., Proc. Natl. Acad. Sci. USA 88:4001-4004 (1991); Milenic, D. E. et al., Cancer Research 51:6363-6371 (1991); Pantoliano, M. W. et al., Biochemistry 30:10117-10125 (1991); Chaudhary, V. K. et al., Nature 339:394-397 (1989); Chaudhary, V. K. et al., Proc. Natl. Acad. Sci. USA 87:1066-1070 (1990); Batra, J. K. et al., Biochem. Biophys. Res. Comm. 171:1-6 (1990); Batra, J. K. et al., J. Biol. Chem. 265:15198-15202 (1990); Chaudhary, V. K. et al., Proc. Natl. Acad. Sci. USA 87:9491-9494 (1990); Batra, J. K. et al., Mol. Cell. Biol. 11:2200-2205 (1991); Brinkmann, U. et al., Proc. Natl. Acad. Sci. USA 88:8616-8620 (1991); Seetharam, S. et al., J. Biol. Chem. 266:17376-17381 (1991); Brinkmann, U. et al., Proc. Natl. Acad. Sci. USA 89:3075-3079 (1992); Glockshuber, R. et al., Biochemistry 29:1362-1367 (1990); Skerra, A. et al., Bio/Technol. 9:273-278 (1991); Pack, P. et al., Biochemistry 31:1579-1534 (1992); Clackson, T. et al., Nature 352:624-628 (1991); Marks, J. D. et al., J. Mol. Biol. 222:581-597 (1991); Iverson, B. L. et al., Science 249:659-662 (1990); Roberts, V. A. et al., Proc. Natl. Acad. Sci. USA 87:6654-6658 (1990); Condra, J. H. et al., J. Biol. Chem. 265:2292-2295 (1990); Laroche, Y. et al., J. Biol. Chem. 266:16343-16349 (1991); Holvoet, P. et al., J. Biol. Chem. 266:19717-19724 (1991); Anand, N. N. et al., J. Biol. Chem. 266:21874-21879 (1991); Fuchs, P. et al., Biol Technol. 9:1369-1372 (1991); Breitling, F. et al., Gene 104:104-153 (1991); Seehaus, T. et al., Gene 114:235-237 (1992); Takkinen, K. et al., Protein Engng. 4:837-841 (1991); Dreher, M. L. et al., J. Immunol. Methods 139:197-205 (1991); Mottez, E. et al., Eur. J. Immunol. 21:467-471 (1991); Traunecker, A. et al., Proc. Natl. Acad. Sci. USA 88:8646-8650 (1991); Traunecker, A. et al., EMBO J. 10:3655-3659 (1991); Hoo, W. F. S. et al., Proc. Natl. Acad. Sci. USA 89:4759-4763 (1993)). Each of the forgoing publications is incorporated herein by reference.

A non-limiting list of targeting groups includes vascular endothelial cell growth factor, FGF2, somatostatin and somatostatin analogs, transferrin, melanotropin, ApoE and ApoE peptides, von Willebrand's Factor and von Willebrand's Factor peptides, adenoviral fiber protein and adenoviral fiber protein peptides, PD1 and PD1 peptides, EGF and EGF peptides, RGD peptides, folate, etc. Other optional targeting agents appreciated by artisans in the art can be also employed in the compounds described herein.

Preferably, the targeting agents include single chain antibody (SCA), RGD peptides, selectin, TAT, penetratin, (Arg)₉, folic acid, etc., and some of the preferred structures of these agents are:

| | | |
|---|---|---|
| C-TAT: | CYGRKKRRQRRR; | (SEQ ID NO: 1) |
| C-(Arg)₉: | CRRRRRRRRR; | (SEQ ID NO: 2) |

RGD can be linear or cyclic:

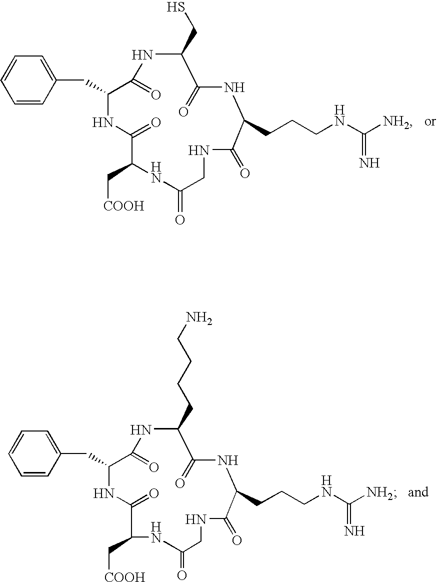

Folic acid is a residue of

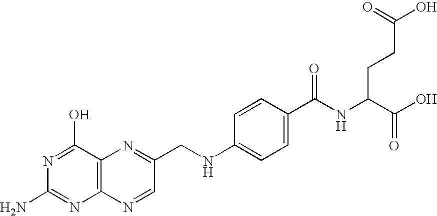

Arg₉ can include a cysteine for conjugating such as CRRRRRRRRR and TAT can add an additional cysteine at the end of the peptide such as CYGRKKRRQRRRC.

For purpose of the current invention, the abbreviations used in the specification and figures represent the following structures:

(i) C-diTAT=CYGRKKRRQRRRYGRKKRRQRRR—NH₂;

(ii) Linear RGD=RGDC;

(iii) Cyclic RGD=c-RGDfC;

(iv) RGD-TAT=CYGRKKRRQRRRGGGRGDS—NH₂; and

(v) Arg₉.

### F. Releasable Linkers

In one preferred aspect of the invention, the compounds described herein contain a biologically active moiety attached to a releasable linker. One advantage of the invention is that the biologically active moiety can be released in a controlled manner.

Among the releasable linkers can be benzyl elimination-based linkers, trialkyl lock-based linkers (or trialkyl lock lactonization based), bicine-based linkers, acid labile linkers, lysosomally cleavable peptides and cathepsin B cleavable peptides. Among the acid labile linkers can be disulfide bond, hydrozone-containing linkers and thiopropionate-containing linkers. Alternatively, the releasable linkers are intracellular labile linkers, extracellular linkers and acidic labile linkers.

The releasable linkers have the formula:

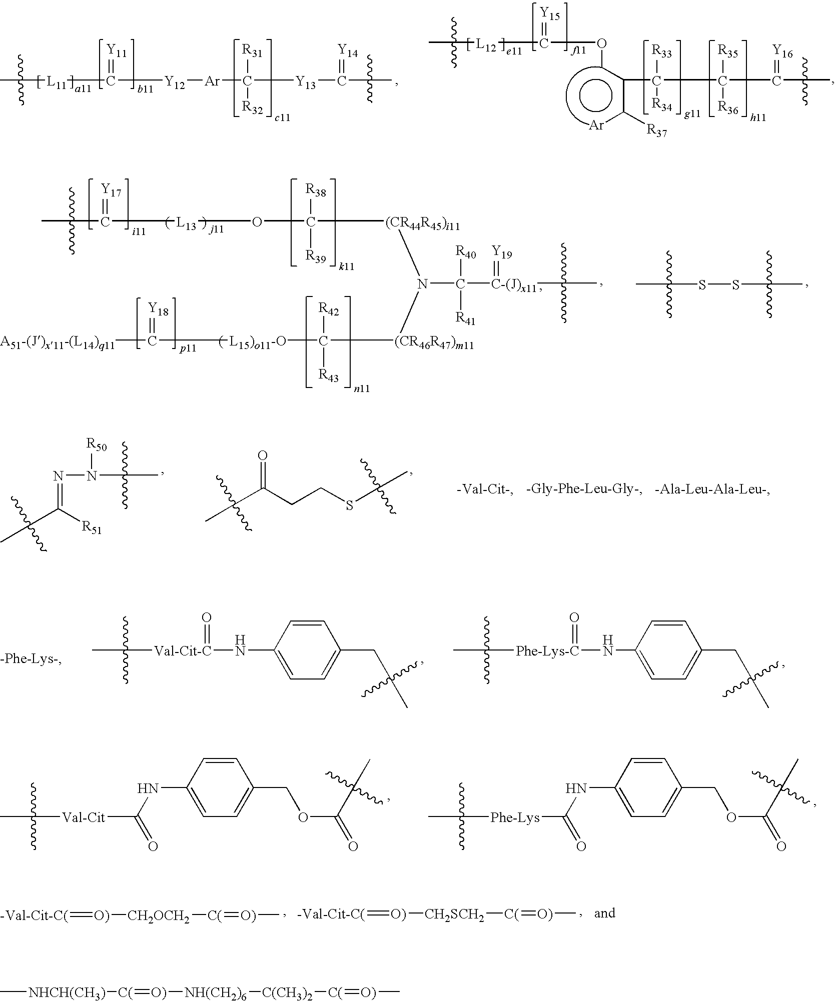

wherein,

Y₁₁₋₁₉ are independently O, S or NR₄₈;

R₃₁₋₄₈, R₅₀₋₅₁ and A₅₁ are independently selected from among hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;

Ar is an aryl or heteroaryl moiety;

L₁₁₋₁₅ are independently selected bifunctional spacers;

J and J′ are independently selected from selected from among moieties actively transported into a target cell, hydrophobic moieties, bifunctional linking moieties and combinations thereof;

(c11), (h11), (k11), (l11), (m11) and (n11) are independently selected positive integers, preferably 1;

(a11), (e11), (g11), (j11), (o11) and (q11) are independently either zero or a positive integer, preferably 1; and

(b11), (x11), (x′11), (f11), (i11) and (p11) are independently zero or one.

Various releasable linkers, benzyl elimination based or trialkyl lock based, are described, for example, in commonly assigned U.S. Pat. Nos. 6,180,095, 6,720,306, 5,965,119, 6,624,142 and 6,303,569, the contents of each of which are incorporated herein by reference. The bicine-based linkers are also described in commonly assigned U.S. Pat. Nos. 7,122,189 and 7,087,229 and U.S. patent application Ser. Nos. 10/557,522, 11/502,108, and 11/011,818, the contents of each of which are incorporated herein by reference.

Preferably, the oligonucleotides are linked to the polymeric portion of the compounds described herein via acid labile linkers. Without being bound by any theory, the acid labile linkers facilitate release of the oligonucleotides from the parent polymeric compounds within cells and specifically in lysosome, endosome, or macropinosome.

In an alternative aspect of the invention, the positively-charged peptides and targeting agents can be also linked to the polymeric portion of the compounds described herein via releasable linkers such as acid labile linkers.

### G. Bifunctional Linkers

In another aspect of the invention, the positively-charged peptides and targeting agents can be linked to the polymeric portion of the compounds described herein via permanent linkers and releasable linkers alone or in combination. Preferably, the positively-charged peptides and targeting agents are linked via permanent linkers.

The bifunctional linkers include amino acids or amino acid derivatives. The amino acids can be among naturally occurring and non-naturally occurring amino acids. Derivatives and analogs of the naturally occurring amino acids, as well as various art-known non-naturally occurring amino acids (D or L), hydrophobic or non-hydrophobic, are also contemplated to be within the scope of the invention. A suitable non-limiting list of the non-naturally occurring amino acids includes 2-aminoadipic acid, 3-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4-aminobutyric acid, desmosine, 2,2-diaminopimelic acid, 2,3-diaminopropionic acid, N-ethylglycine, N-ethylasparagine, 3-hydroxyproline, 4-hydroxyproline, isodesmosine, allo-isoleucine, N-methylglycine, sarcosine, N-methyl-isoleucine, 6-N-methyl-lysine, N-methylvaline, norvaline, norleucine, and ornithine. Some preferred amino acid residues include glycine, alanine, methionine and sarcosine.

Alternatively, the bifunctional linkers can be selected from among

—[C(═O)]ᵥ(CR₂₂R₂₃)ₜ[C(═O)]_{v′}—,

—[C(═O)]ᵥ(CR₂₂R₂₃)ₜ—O[C(═O)]_{v′}—,

—[C(═O)]ᵥ(CR₂₂R₂₃)ₜ—NR₂₆[C(═O)]_{v′}—,

—[C(═O)]ᵥO(CR₂₂R₂₃)ₜ[C(═O)]_{v′}—,

—[C(═O)]ᵥO(CR₂₂R₂₃)ₜO[C(═O)]_{v′}—,

—[C(═O)]ᵥO(CR₂₂R₂₃)ₜNR₂₆[C(═O)]_{v′}—,

—[C(═O)]ᵥNR₂₁(CR₂₂R₂₃)ₜ[C(═O)]_{v′}—,

—[C(═O)]ᵥNR₂₁(CR₂₂R₂₃)ₜO[C(═O)]_{v′}—,

—[C(═O)]ᵥNR₂₁(CR₂₂R₂₃)ₜNR₂₆[C(═O)]_{v′}—,

—[C(═O)]ᵥ(CR₂₂R₂₃)ₜO—(CR₂₈R₂₉)_{t′}[C(═O)]_{v′}—,

—[C(═O)]ᵥ(CR₂₂R₂₃)ₜNR₂₆—(CR₂₈R₂₉)_{t′}[C(═O)]_{v′}—,

—[C(═O)]ᵥ(CR₂₂R₂₃)ₜS—(CR₂₈R₂₉)_{t′}[C(═O)]_{v′}—,

—[C(═O)]ᵥO(CR₂₂R₂₃)ₜO—(CR₂₈R₂₉)_{t′}[C(═O)]_{v′}—,

—[C(═O)]ᵥO(CR₂₂R₂₃)ₜNR₂₆—(CR₂₈R₂₉)_{t′}[C(═O)]_{v′}—,

—[C(═O)]ᵥO(CR₂₂R₂₃)ₜS—(CR₂₈R₂₉)_{t′}[C(═O)]_{v′}—,

—[C(═O)]ᵥ NR₂₁(CR₂₂R₂₃)ₜO—(CR₂₈R₂₉)_{t′}[C(═O)]_{v′}—,

—[C(═O)]ᵥNR₂₁(CR₂₂R₂₃)ₜNR₂₆—(CR₂₈R₂₉)_{t′}[C(═O)]_{v′}—,

—[C(═O)]ᵥNR₂₁(CR₂₂R₂₃)ₜS—(CR₂₈R₂₉)_{t′}[C(═O)]_{v′}—,

—[C(═O)]ᵥ(CR₂₂R₂₃CR₂₈R₂₉O)ₜNR₂₆[C(═O)]_{v′}—,

—[C(═O)]ᵥ(CR₂₂R₂₃CR₂₈R₂₉O)ₜ[C(═O)]_{v′}—,

—[C(═O)]ᵥO(CR₂₂R₂₃CR₂₈R₂₉O)ₜNR₂₆[C(═O)]_{v′}—,

—[C(═O)]ᵥO(CR₂₂R₂₃CR₂₈R₂₉O)ₜ[C(═O)]_{v′}—,

—[C(═O)]ᵥNR₂₁(CR₂₂R₂₃CR₂₈R₂₉O)ₜNR₂₆[C(═O)]_{v′}—,

—[C(═O)]ᵥNR₂₁(CR₂₂R₂₃CR₂₈R₂₉O)ₜ[C(═O)]_{v′}—,

—[C(═O)]ᵥ(CR₂₂R₂₃CR₂₈R₂₉O)ₜ(CR₂₄R₂₅)_{t′}[C(═O)]_{v′}—,

—[C(═O)]ᵥO(CR₂₂R₂₃CR₂₈R₂₉O)ₜ(CR₂₄R₂₅)_{t′}[C(═O)]_{v′}—,

—[C(═O)]ᵥNR₂₁(CR₂₂R₂₃CR₂₈R₂₉O)ₜ(CR₂₄R₂₅)_{t′}[C(═O)]_{v′}—,

—[C(═O)]ᵥ(CR₂₂R₂₃CR₂₈R₂₉O)ₜ(CR₂₄R₂₅)_{t′}O[C(═O)]_{v′}—,

—[C(═O)]ᵥ(CR₂₂R₂₃)ₜ(CR₂₄R₂₅CR₂₈R₂₉O)_{t′}[C(═O)]_{v′}—,

—[C(═O)]ᵥ(CR₂₂R₂₃)ₜ(CR₂₄R₂₅CR₂₈R₂₉O)_{t′}NR₂₆[C(═O)]_{v′}—,

—[C(═O)]ᵥO(CR₂₂R₂₃CR₂₈R₂₉O)ₜ(CR₂₄R₂₅)_{t′}O[C(═O)]_{v′}—,

—[C(═O)]ᵥO(CR₂₂R₂₃)ₜ(CR₂₄R₂₅CR₂₈R₂₉O)_{t′}[C(═O)]_{v′—,}

—[C(═O)]ᵥO(CR₂₂R₂₃)ₜ(CR₂₄CR₂₅CR₂₈R₂₉O)_{t′}NR₂₆[C(═O)]_{v′}—,

—[C(═O)]ᵥNR₂₁(CR₂₂R₂₃CR₂₈R₂₉O)ₜ(CR₂₄R₂₅)_{t′}O[C(═O)]_{v′}—,

—[C(═O)]ᵥNR₂₁(CR₂₂R₂₃)ₜ(CR₂₄R₂₅CR₂₈R₂₉O)_{t′}[C(═O)]_{v′—,}

—[C(═O)]ᵥNR₂₁(CR₂₂R₂₃)ₜ(CR₂₄R₂₅CR₂₈R₂₉O)_{t′}NR₂₆[C(═O)]_{v′—,}

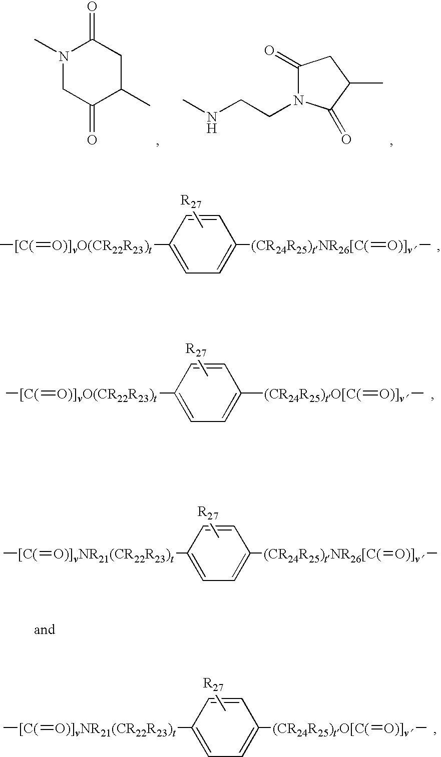

wherein:

R₂₁₋₂₉ are independently selected from among hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;

(t) and (t′) are independently zero or a positive integer, preferably zero or an integer from about 1 to about 12, more preferably an integer from about 1 to about 8, and most preferably 1 or 2; and

(v) and (v′) are independently zero or 1.

Preferably, the bifunctional linkers can be selected from among:

—[C(═O)]ᵣNH(CH₂)₂CH═N—NHC(═O)—(CH₂)₂—,

—[C(═O)]ᵣNH(CH₂)₂(CH₂CH₂O)₂(CH₂)₂NH[C(═O)]_{r′}—,

—[C(═O)]ᵣNH(CH₂CH₂)(CH₂CH₂O)₂NH[C(═O)]_{r′}—,

—[C(═O)]ᵣNH(CH₂CH₂)ₛNH(CH₂CH₂)_{s′}[C(═O)]_{r′}—,

—[C(═O)]ᵣNH(CH₂CH₂)ₛS(CH₂CH₂)_{s′}[C(═O)]_{r′}—,

—[C(═O)]ᵣNH(CH₂CH₂)(CH₂CH₂O)[C(═O)]_{r′}—,

—[C(═O)]ᵣNH(CH₂CH₂)ₛO(CH₂CH₂)_{s′}[C(═O)]_{r′}—,

—[C(═O)]ᵣNH(CH₂CH₂O)(CH₂)NH[C(═O)]_{r′}—,

—[C(═O)]ᵣNH(CH₂CH₂O)₂(CH₂)[C(═O)]_{r′}—,

—[C(═O)]ᵣNH(CH₂CH₂O)ₛ(CH₂)_{s′}[C(═O)]_{r′}—,

—[C(═O)]ᵣNHCH₂CH₂NH[C(═O)]_{r′}—,

—[C(═O)]ᵣNH(CH₂CH₂)₂O[C(═O)]_{r′}—,

—[C(═O)]ᵣNH(CH₂CH₂O)[C(═O)]_{r′}—,

—[C(═O)]ᵣNH(CH₂CH₂O)₂[C(═O)]_{r′}—,

—[C(═O)]ᵣNH(CH₂)₃[C(═O)]_{r′}—,

—[C(═O)]ᵣO(CH₂CH₂O)₂(CH₂)[C(═O)]_{r′}—,

—[C(═O)]ᵣO(CH₂)₂NH(CH₂)₂[C(═O)]_{r′}—,

—[C(═O)]ᵣO(CH₂CH₂O)₂NH[C(═O)]_{r′}—,

—[C(═O)]ᵣO(CH₂)₂O(CH₂)₂[C(═O)]_{r′}—,

—[C(═O)]ᵣO(CH₂)₂S(CH₂)₂[C(═O)]_{r′}—,

—[C(═O)]ᵣO(CH₂CH₂)NH[C(═O)]_{r′}—,

—[C(═O)]ᵣO(CH₂CH₂)O[C(═O)]_{r′}—,

—[C(═O)]ᵣO(CH₂)₃NH[C(═O)]_{r′}—,

—[C(═O)]ᵣO(CH₂)₃O[C(═O)]_{r′}—,

—[C(═O)]ᵣO(CH₂)₃[C(═O)]_{r′}—,

—[C(═O)]ᵣO(CH₂NHCH₂[C(═O)]_{r′}—,

—[C(═O)]ᵣCH₂OCH₂ [C(═O)]_{r′}—,

—[C(═O)]ᵣCH₂SCH₂[C(═O)]_{r′}—,

—[C(═O)]ᵣS(CH₂)₃[C(═O)]_{r′}—,

—[C(═O)]ᵣ(CH₂)₃[C(═O)]_{r′}—,

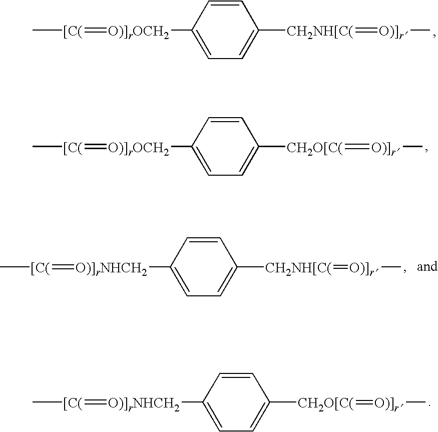

wherein (r) and (r′) are independently zero or 1, provided that both (r) and (r′) are not simultaneously zero.

In yet further alternative aspects of the invention, the bifunctional linkers include:

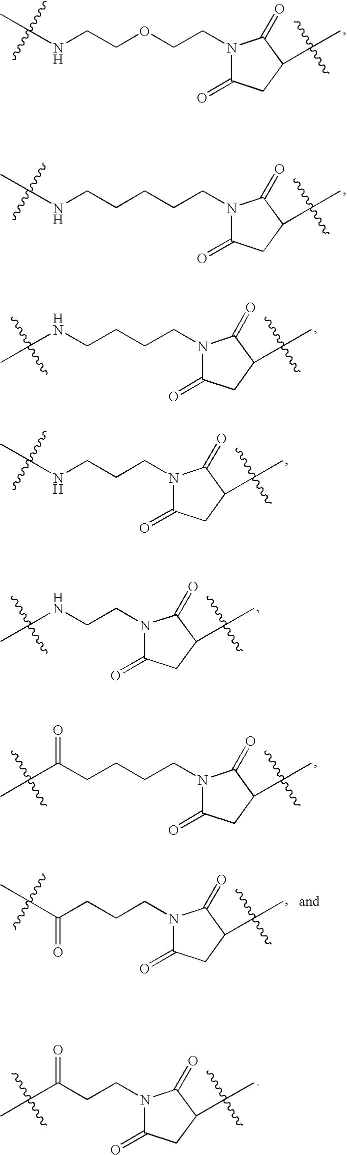

These bifunctional groups allow a second agent to be directly conjugated and therefore eliminate the need of attaching a functional group for conjugating to a second agent.

In an alternative embodiment, the bifunctional linkers include structures corresponding to those shown above but instead of maleimidyl group have groups such as vinyl, residues of sulfone, amino, carboxy, mercapto, thiopropionate, hydrazide, carbazate and the like instead of maleimidyl.

### H. Branching Groups

Polymer arm terminals of the compounds described herein can be branched for allowing multiple loading of biologically active moieties, positively charged moieties and/or targeting agents. Preferably, the branching groups provide more polymer arm terminals available for positively-charged moieties.

The branching groups can have at least three functional sites. The number of polymer arm terminals is multiplied by the degree of branching. When a branching group having three functional sites is linked to the polymeric compounds, it provides two terminals for conjugation. The branching groups can be selected among:

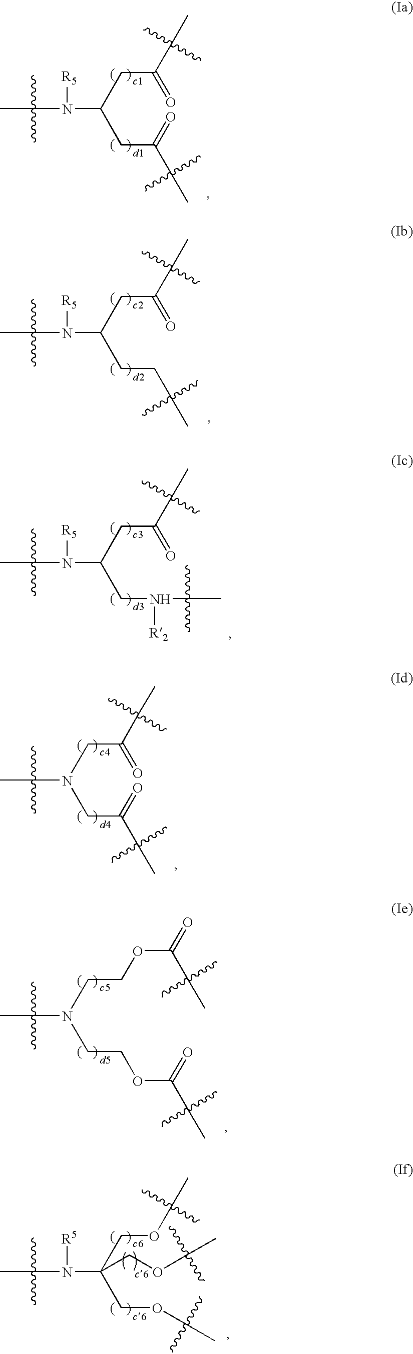

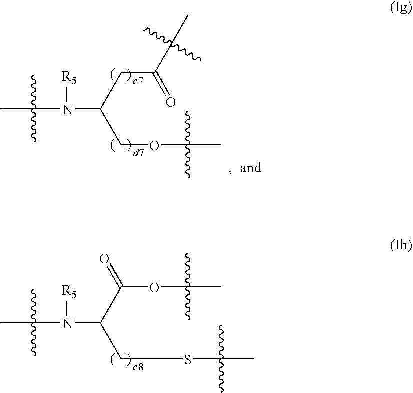

wherein

R₅ is independently selected from among hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, and substituted arylcarbonyloxy;

(c1), (c2), (c3), (c4), (c5), (c6), (c′6), (c″6), (c7) and (c8) are independently zero or a positive integer, preferably zero or an integer from about 1 to 10, and more preferably zero, 1 or 2; and

(d1), (d2), (d3), (d4), (d5) and (d7) are independently zero or a positive integer, preferably zero or an integer from about 1 to about 10, and more preferably zero or an integer from about 1 to about 4.

Various branching groups are also described in commonly owned U.S. Pat. Nos. 6,153,655, 6,395,266 and 6,638,499, the contents of each of which are incorporated herein by reference. All other optional branching groups known to those of ordinary skill are also contemplated as being within the compounds described herein.

Preferably, the branching groups include:

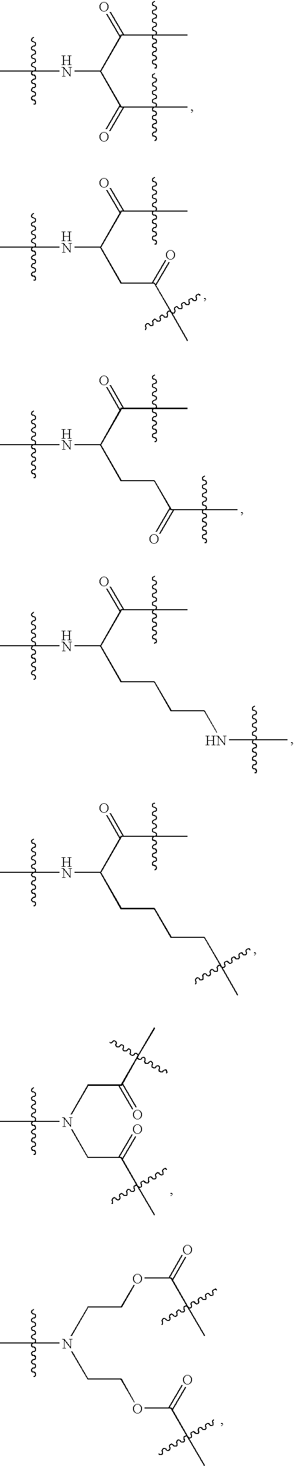

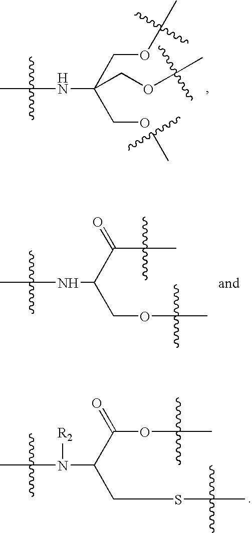

More preferably, the branching group includes aspartic acid, glutamic acid, lysine, and cysteine.

In a further aspect, one or more branching groups can be employed at each terminal of the polymer arms.

### I. Preferred Embodiments Corresponding to Formula I

In one preferred embodiment, the polymeric compounds have the formulae:

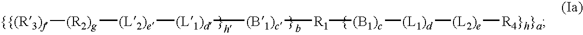

wherein

(e) is 1 or 2;

(e′) is 0, 1 or 2; and

(f′) is 0 or 1,

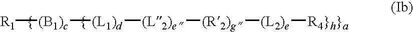

wherein (g′) is a positive integer.

For example, the conjugates prepared in accordance with the present invention are among:

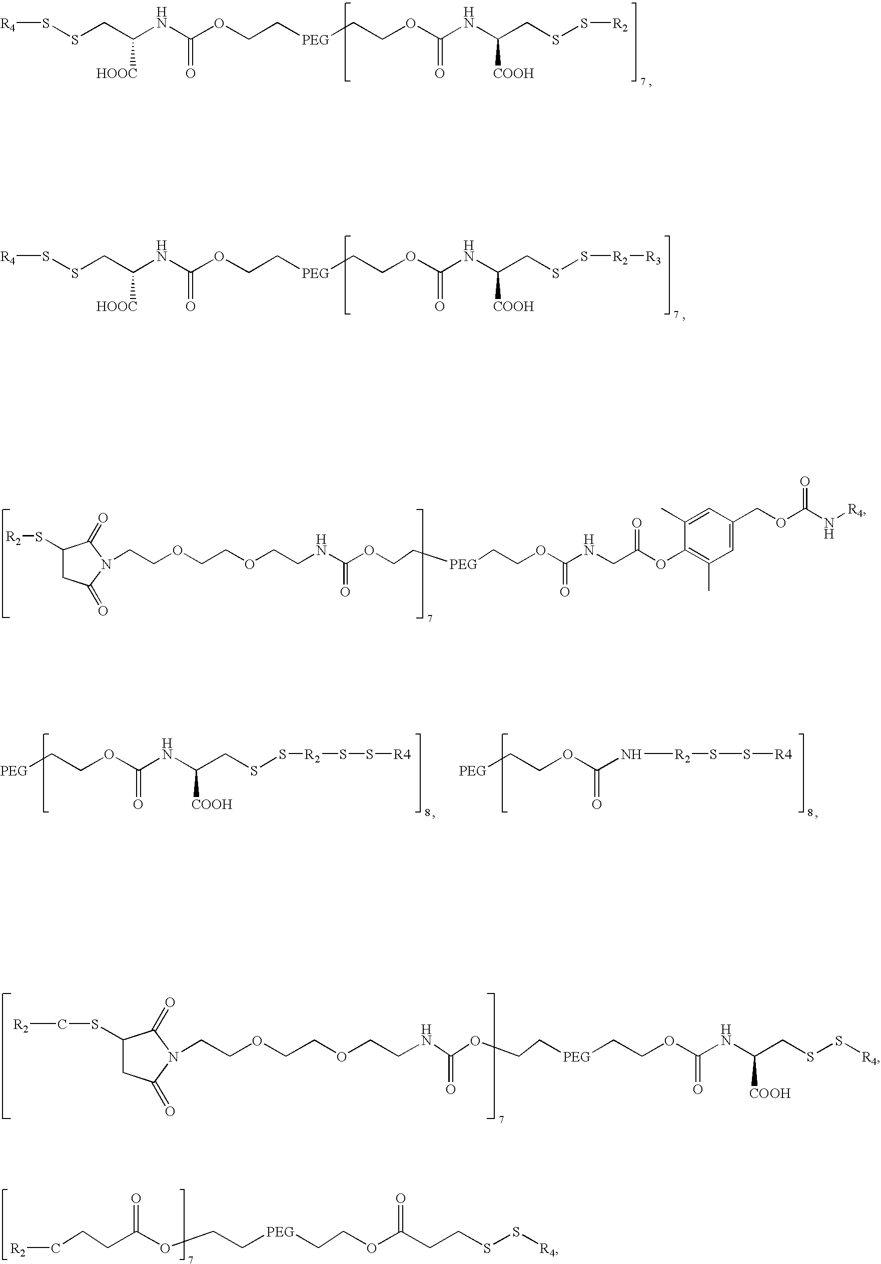

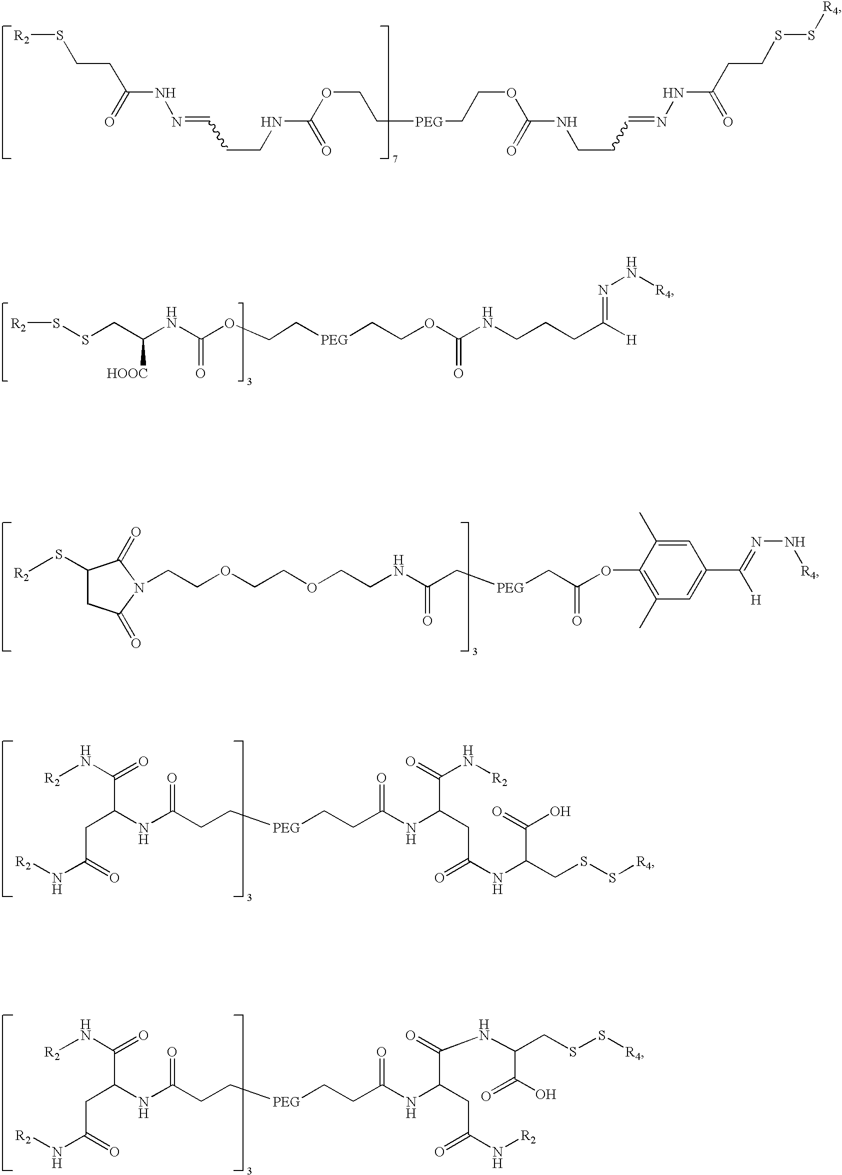

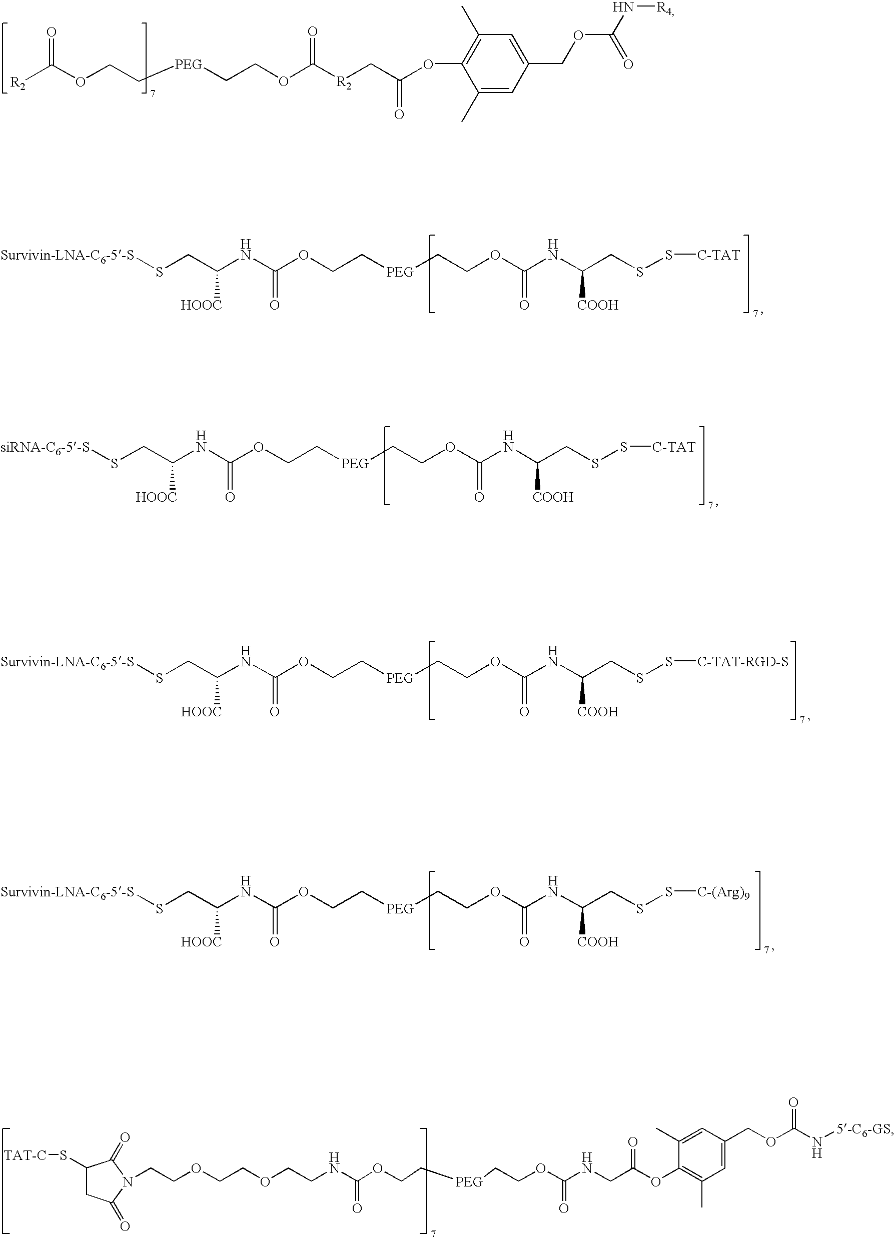

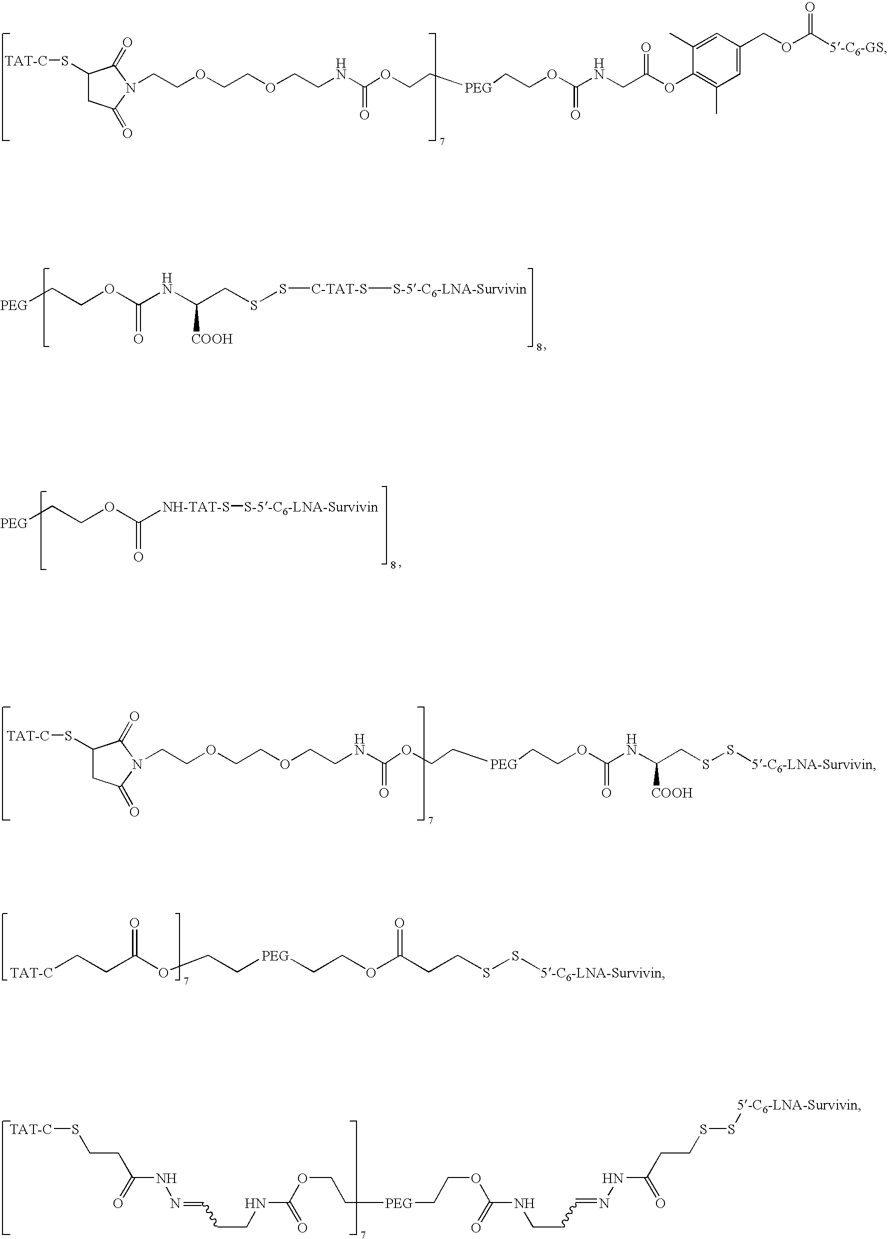

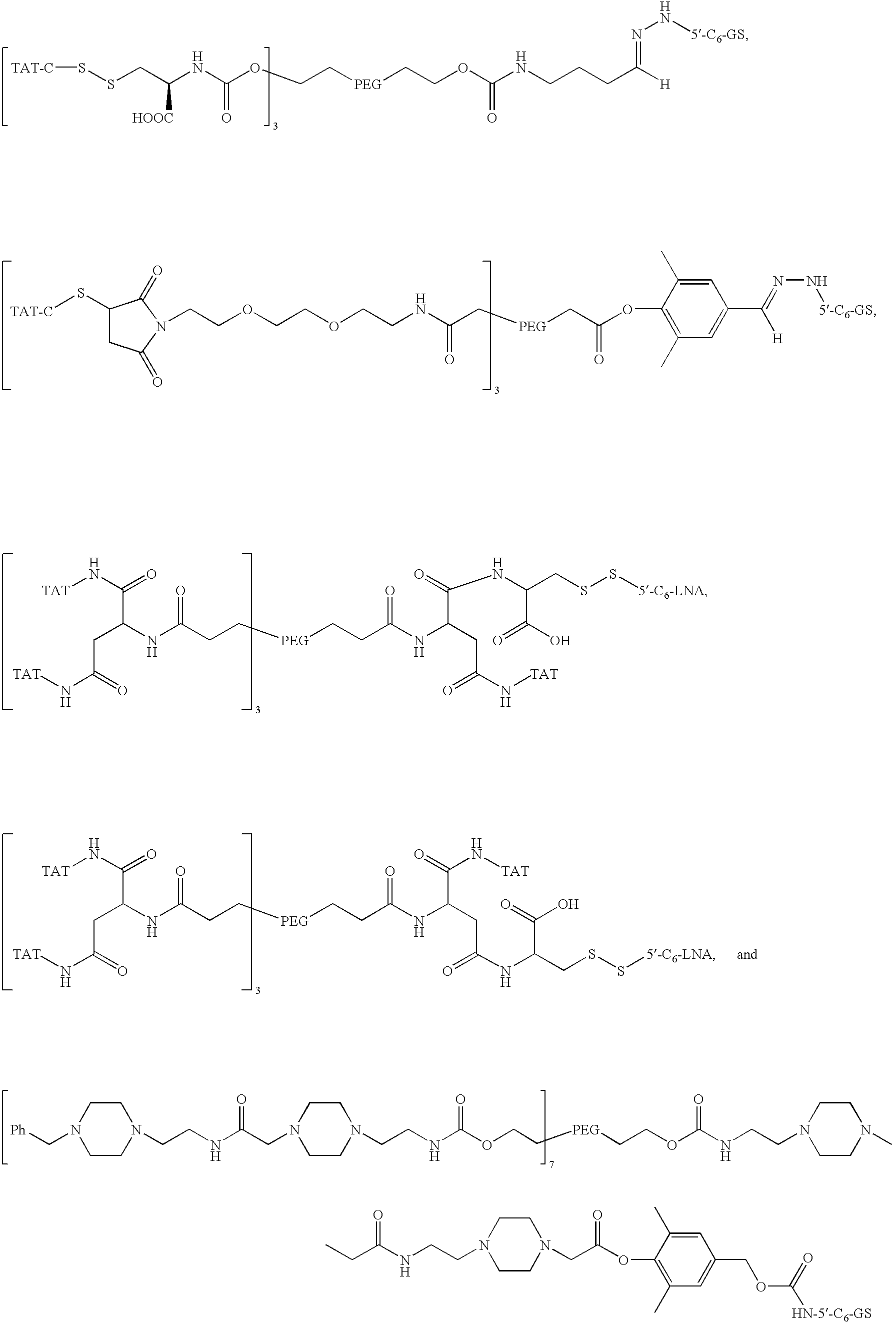

wherein

C-TAT is a residue of —S—CYGRKKRRQRRR—CONH₂;

NH-5′-C₆-GS is derivative of Genasense, an 18-mer phosphorothioate antisense oligonucleotide TCTCCCAGCGTGCGCCAT (SEQ ID NO: 1)

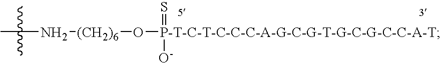

S-5′-C-LNA-Survivin is

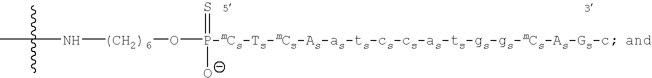

RGD is

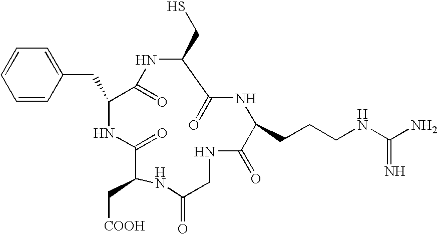

In one preferred embodiment of the invention, the polymeric compounds include:

The 5′-end of the sense strand of the siRNA duplex is modified to a C6-amino tail for conjugating to PEG linkers.

### J. Synthesis of the Polymeric Delivery Systems

Generally, the conjugates can be made by sequentially attaching the polymer, cytotoxic agent, positive-charge containing moiety, and targeting moiety to the multifunctional linker. The exact order of addition is not limited to this order and as will be apparent to those of ordinary skill, there are aspects in which the PEG will be first added to the multifunctional linker followed by the addition of the releasably attached cytotoxic drug followed by the addition of the positive-charge containing moiety and targeting agent like the monoclonal antibody. Details concerning some preferred aspects of this embodiment are provided in the Examples below.

In one aspect of the invention, a polymeric compound containing a OH or a leaving group can first react with a nucleophile containing a releasable linker moiety, and then react with another nucleophile containing a functional group at the distal end. The releasable linker can conjugate with a biologically active compound and the functional group can link to a positive-charge containing moieties. Alternatively, the polymeric compound conjugated to a biologically active moiety and positive-charge containing moieties can further react with a targeting moiety to prepare the final polymeric conjugate containing all three component of the invention. For example, the artisan can use less equivalent of the nucleophile compare to the number of the leaving groups on the polymer to form a polymeric intermediate containing both linker and leaving group. This intermediate can further reacted with a positive-charge containing moiety and alternatively, further with a targeting moiety to form the polymeric conjugate multisubstituted with biologically active compound, positive-charge containing moiety, and a targeting agent.

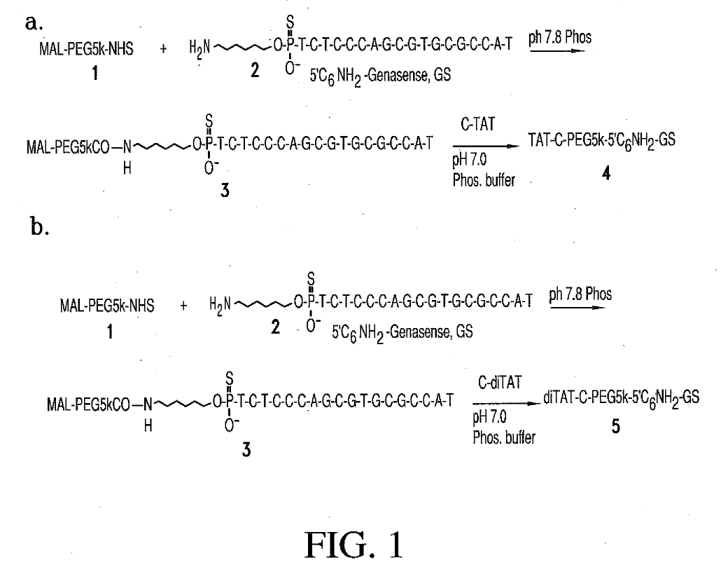

Alternatively, the polymer can be activated with different groups to provide different chemical reactivities toward various nucleophilic moieties. For example, different protecting groups such as tert-Bu ester and methyl ester of carboxylic acid terminals can be deprotected selectively and stepwise to provide various degrees of active group to be conjugated with different biologically active agents such as cytotoxic agent and targeting agent. As shown in FIG. 1, maleimidyl group and succinimidyl ester can react selectively with SH or NH₂ containing moieties, respectively.

All reactions described herein are standard chemical reactions with necessary steps and conditions known to those of an ordinary skill. The synthetic reactions described herein therefore do not require undue experimentation.

The attachment of the nucleophilic compound to the PEG or other polymer can be done using standard chemical synthetic techniques well known to those of ordinary skill. The activated polymer portion such as SC-PEG, PEG-amine, PEG acids, etc. can be obtained from either commercial sources or synthesized by the artisan without undue experimentation.

Attachment of nucleophilic compound to the polymer portion is alternatively carried out in the presence of a coupling agent. A non-limiting list of suitable coupling agents include 1,3-diisopropylcarbodiimide (DIPC), any suitable dialkyl carbodiimides, 2-halo-1-alkyl-pyridinium halides (Mukaiyama reagents), 1-(3-dimethylaminopropyl)-3-ethyl carbodiimide (EDC), propane phosphonic acid cyclic anhydride (PPACA) and phenyl dichlorophosphates, etc. which are available, for example from commercial sources such as Sigma-Aldrich Chemical, or synthesized using known techniques.

Preferably, the reactions are carried out in an inert solvent such as methylene chloride, chloroform, DMF or mixtures thereof. The reactions can be preferably conducted in the presence of a base, such as dimethylaminopyridine (DMAP), diisopropylethylamine, pyridine, triethylamine, etc. to neutralize any acids generated. The reactions can be carried out at a temperature from about 0° C. up to about 22° C. (room temperature). Some particular embodiments prepared by the methods described herein include:

In one aspect, the polymeric compounds with positively-charged moieties to neutralize the negative charge and improved the cellular uptake of biologically active moieties such as oligonucleotides can have the following alternative aspects:

(i) oligonucleotides modified with (CH₂)_{w} amino linkers at 5′- or 3′-end of the oligonucleotides;

(ii) oligonucleotides modified with (CH₂)_{w} sulfhydryl linkers at 5′- or 3′-end of the oligonucleotides;

(iii) oligonucleotides modified with (CH₂)_{w} amino linkers or (CH₂)_{w} sulfhydryl linkers containing hindered ester, which can release the oligonucleotides without amino tail or thio tail;

(iv) one or more positively-charged peptides, for example, two positively-charged peptides such as TAT sequences can be attached for enhancing cellular uptake;

(v) one or more releasable linkers can be attached

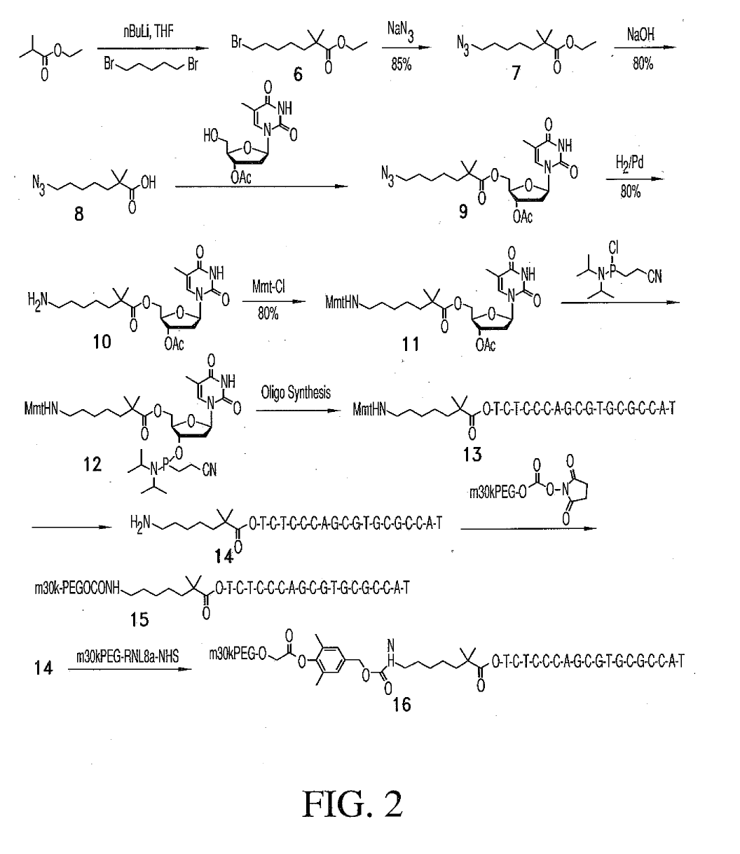

Description concerning the formation of hindered ester-containing oligonucleotides is described in commonly-assigned U.S. Provisional Patent Application No. 60/845,028, entitled “Hindered Ester-Based Biodegradable Linkers For Oligonucleotide Delivery”, the contents of which are incorporated herein by reference. See the reaction scheme in FIG. 2.

### K. Methods of Treatment

In view of the above, there are also provided methods of treating a mammal, comprising administering an effective amount of a pharmaceutical composition containing a compound of the present invention of Formula (I) to a patient in need thereof.

In one particular aspect of the invention, there are also provided methods of treating a patient having a malignancy or cancer, comprising administering an effective amount of a pharmaceutical composition containing the compound of Formula (I) to a patient in need thereof. In alternative aspects, the cancer being treated can be one or more of the following: solid tumors, lymphomas, small cell lung cancer, acute lymphocytic leukemia (ALL), pancreatic cancer, glioblastoma, ovarian cancer, gastric cancers, etc. The compositions are useful for treating neoplastic disease, reducing tumor burden, preventing metastasis of neoplasms and preventing recurrences of tumor/neoplastic growths in mammals.

Another aspect of the present invention provides methods of treatment for various medical conditions in mammals. Briefly stated, any biologically active moiety which can be attached to the positively charged PEG polymer can be administered to a mammal in need of such treatment Any oligonucleotide, etc. which has therapeutic effects in the unconjugated state can be used in its conjugated form, made as described herein.

The amount of the composition, e.g., used as a prodrug, that is administered will depend upon the parent molecule included therein. Generally, the amount of prodrug used in the treatment methods is that amount which effectively achieves the desired therapeutic result in mammals. Naturally, the dosages of the various prodrug compounds will vary somewhat depending upon the parent compound, rate of in vivo hydrolysis, molecular weight of the polymer, etc.

In a further aspect of the invention, there are provided methods of administering polynucleotides (oligonucleotides), preferably antisense oligonucleotides to mammalian cells. The methods include delivering an effective amount of a conjugate prepared as described herein to the condition being treated will depend upon the polynucleotides efficacy for such conditions. For example, if the unconjugated oligonucleotides (for example antisense BCL2 oligonucleotides, antisense Survivin oligonucleotides) has efficacy against certain cancer or neoplastic cells, the method would include delivering a polymer conjugate containing the oligonucleotides to the cells having susceptibility to the native oligonucleotides. The delivery can be made in vivo as part of a suitable pharmaceutical composition or directly to the cells in an ex vivo environment. In one particular treatment, the polymeric conjugates including oligonucleotides (SEQ ID NO. 3, SEQ ID NOs: 4 and 5, and SEQ ID NO: 6, and SEQ ID NO: 7) can be used.

### EXAMPLES

The following examples serve to provide further appreciation of the invention but are not meant in any way to restrict the scope of the invention. The bold-faced numbers recited in the Examples correspond to those shown in FIG. 1. Abbreviations are used throughout the examples such as, DCM (dichloromethane), DIEA (diisopropylethylamine), DMAP (4-dimethylaminopyridine), DMF (N,N′-dimethylformamide), DSC (disuccinimidyl carbonate), EDC (1-(3-dimethylaminopropyl)-3-ethyl carbodiimide), IPA (isopropanol), NHS (N-hydroxysuccinimide), PEG (polyethylene glycol), SCA-SH (single-chain antibody), SN38 (7-ethyl-10-hydroxycamptothecin), TBDPS (tert-butyl-dipropylsilyl), and TEA (triethylamine).

General Procedures. All reactions are run under an atmosphere of dry nitrogen or argon. Commercial reagents are used without further purification. All PEG compounds are dried in vacuo or by azeotropic distillation from toluene prior to use. ¹H NMR spectra were obtained at 300 MHz and ¹³C NMR spectra at 75.46 MHz using a Varian Mercury 300 NMR spectrometer and deuterated chloroform as the solvents unless otherwise specified. Chemical shifts (5) are reported in parts per million (ppm) downfield from tetramethylsilane (TMS).

HPLC Method. The reaction mixtures and the purity of intermediates and final products are monitored by a Beckman Coulter System Gold® HPLC instrument. It employs a ZORBAX® 300SB C8 reversed phase column (150×4.6 mm) or a Phenomenex Jupiter® 300A C18 reversed phase column (150×4.6 mm) with a 168 Diode Array UV Detector, using a gradient of 10-90% of acetonitrile in 0.05% trifluoroacetic acid (TFA) at a flow rate of 1 mL/min.)

### Example 1

### Compound 3

To a solution of compound 2 (10 mg, 1.7 μmol) in PBS buffer (5 mL, pH 7.8) was added Mal-PEG5k-NHS from NOF corp. (100 mg, 17 mmol) and stirred at room temperature for 2 hours. The reaction mixture was diluted to 20 mL with water and loaded on a Poros HQ, strong anion exchange column (10 mm×1.5 mm, bed volume ˜16 mL) which was pre-equilibrated with 20 mM Tris-HCl buffer, pH 7.0 (buffer A). The column was washed with 3-4 column volumes of buffer A to remove the excess PEG linker. Then the product was eluted with a gradient of 0 to 100% 1 M NaCl in 20 mM Tris-HCl buffer, pH 7.0, buffer B in 10 minutes, followed by 100% buffer B for 10 minutes at a flow rate of 10 mL/min. The eluted product was desalted using HiPrep desalting column (50 mL) and lyophilized to give compound 3. Yield 6 mg (oligo equivalent, 60%).

### Example 2

### Compound 4

To a solution of compound 3 in PBS buffer (6 mL, pH 7.0), peptide C-Tat (5 mg, 3 μmol) was added and stirred at room temperature for 2 hours. The reaction mixture was diluted to 20 mL with water and loaded on a Resource S, strong cation-exchange column (10 mm×1.5 mm, bed volume ˜16 mL) which was pre-equilibrated with 100 mM K₂ HPO₄, 5M urea buffer, pH 6.5 (buffer A). The column was washed with 3-4 column volumes of buffer A to remove the unreacted PEG-oligo compound. Then the product was eluted with a gradient of 0 to 100% 2 M KBr (buffer B) in 10 minutes, followed by 100% buffer B for 10 minutes at a flow rate of 10 mL/min. The eluted product was desalted using HiPrep desalting column (50 mL) and lyophilized to give compound 4. Yield 2 mg (oligo equivalent, 30%).

### Example 3

### Compound 5

To a solution of compound 3 in PBS buffer (6 mL, pH 7.0), peptide C-diTat (10 mg, 3 μmol) was added and stirred at room temperature for 2 hours. The reaction mixture was diluted to 20 mL with water and loaded on a Resource S, strong cation-exchange column (10 mm×1.5 mm, bed volume ˜16 mL) which was pre-equilibrated with 100 mM K₂HPO₄, 5M urea buffer, pH 6.5 (buffer A). The column was washed with 3-4 column volumes of buffer A to remove the unreacted PEG-oligo compound. Then the product was eluted with a gradient of 0 to 100% 2 M KBr (buffer B) in 10 minutes, followed by 100% buffer B for 10 minutes at a flow rate of 10 mL/min. The eluted product was desalted using HiPrep desalting column (50 mL) and lyophilized to give compound 5. Yield 2 mg (oligo equivalent, 30%).

### Example 4

### Compound 6

Butyllithium (1.6 M, 200 mL) was added to a solution of ethyl isobutyrate (35 g) in THF (500 mL) at −78° C. and the solution was stirred for 1 hour at the same temperature. 1,5-Dibromopetane (100 g) was added and the mixture was allowed to warm up to room temperature. The mixture was stirred at room temperature for 1 hour and was poured into aqueous sodium bicarbonate (500 mL). The organic layer was evaporated. The residue was purified by a silica gel column, eluted with 10% ethyl acetate in hexane to give compound 6 as a liquid (29.2 g, yield 36.7%).

### Example 5

### Compound 7

Ethyl 7-bromo-2,2-dimethylheptanoate (compound 6, 26.5 g) was heated with sodium azide (13 g) in DMF (500 mL) at 100° C. for 2 hour. The mixture was concentrated and the residue was purified by a silica gel column, eluted with 10% ethyl acetate in hexane to give the compound 7 as a liquid (20.5 g, yield 90.3%).

### Example 6

### Compound 8

Ethyl 7-azido-2,2-dimethylheptanoate (compound 7, 20.5 g) was heated with sodium hydroxide (10 g, 85%) in ethanol (500 mL) under reflux for 2 hours. The mixture was concentrated and water (400 mL) was added. The mixture was acidified with concentrated hydrochloric acid to pH 2 and extracted with ethyl acetate (500 mL). The organic layer was concentrated and the residue was purified by a silica gel column, eluted with 50% ethyl acetate in hexane to give compound 8 as a liquid (17.1 g, yield 95%).

### Example 7

### Compound 9

7-Azido-2,2-dimethylheptanoic acid (compound 8, 8 g) was dissolved in dichloromethane (200 mL). Oxalyl chloride (6.4 g) was added and the mixture was refluxed for 2 hours and evaporated. The residue was dissolved in dichloromethane (100 mL) and was added in 3′-acetyl thymidine (5.85 g) in pyridine (100 mL). The solution was stirred at room temperature for 24 hours and was poured into aqueous sodium bicarbonate (500 mL). The mixture was extracted with dichloromethane (500 mL) and the organic layer was concentrated. The residue was purified by a silica gel column, eluted with 5% methanol in dichloromethane to give compound 9 as a colorless solid (5.6 g, yield 61%).

### Example 8

### Compound 10

5′-(7-Azido-2,2-dimethylheptanoyl)-3′-acetylthymidine (compound 9, 4.65 g) was hydrogenated in methanol (200 mL) under 30 psi in the presence of Pd/C (10%, 0.5 g) for 1 hour. The mixture was filtered and the filtrate was evaporated to give compound 10 as a solid (4.4 g, yield 100%).

### Example 9

### Compound 11

5′-(7-Amino-2,2-dimethylheptanoyl) 3′-acetylthymidine (compound 10, 4.4 g), triethylamine (4 mL) and 4-methoxytrityl chloride (7.5 g) were stirred in pyridine (100 mL) for 10 hour. Methylamine (40%, 10 mL) was added and the solution was stirred for 2 hour. The mixture was poured into aqueous sodium bicarbonate (500 mL) and extracted with dichloromethane (500 ml). The organic layer was concentrated. The residue was purified by a silica gel column, eluted with 5% methanol in dichloromethane to give compound 11 as a colorless solid (4.9 g, yield 71%).

### Example 10

### Compound 12

5′-(7-[(MMT-amino)-2,2-dimethylheptanoyl]thymidine (compound 11, 4.9 g), N,N-tetraisopropyl-cyanoethyl phosphoramidite (3 g) and tetrazole (0.5 g) in acetonitrile (50 mL) was stirred overnight. The mixture was poured into aqueous sodium bicarbonate (500 ml) and extracted with dichloromethane (500 mL). The organic layer was concentrated. The residue was purified by a silica gel column, eluted with 50% ethyl acetate in hexane to give compound 12 as a colorless solid (4.5 g, yield 71%).

### Example 11

### Compounds 14

Compound 12 was transferred to Trilink Biotechnologies, CA to use as the last monomer in the oligo synthesis. The Mmt group was deprotected after the synthesis and the oligo was purified by RP-HPLC and compound 14 as the free amine was obtained for PEG conjugation

### Example 12

### Compound 15

To a solution of compound 14 (10 mg, 1.7 μmol) in PBS buffer (5 mL, pH 7.8) was added m30kSCPEG (520 mg, 17 μmol) and stirred at room temperature for 5 hours. The reaction mixture was diluted to 50 mL with water and loaded on a Poros HQ, strong anion exchange column (10 mm×1.5 mm, bed volume ˜16 mL) which was pre-equilibrated with 20 mM Tris-HCl buffer, pH 7.4 (buffer A). The column was washed with 3-4 column volumes of buffer A to remove the excess PEG linker. Then the product was eluted with a gradient of 0 to 100% 1 M NaCl in 20 mM Tris-HCl buffer, pH 7.4, buffer B in 10 minutes, followed by 100% buffer B for 10 minutes at a flow rate of 10 mL/min. The eluted product was desalted using HiPrep desalting column (50 mL) and lyophilized to give compound 15. Yield 6 mg (oligo equivalent, 60%).

### Example 13

### Compound 16

To a solution of compound 14 (10 mg, 1.7 μmol) in PBS buffer (5 mL, pH 7.8) was added m30PEG-RNL8a-NHS (520 mg, 17 μmol) and stirred at room temperature for 5 hours. The reaction mixture was diluted to 50 mL with water and loaded on a Poros HQ, strong anion exchange column (10 mm×1.5 mm, bed volume ˜16 mL) which was pre-equilibrated with 20 mM Tris-HCl buffer, pH 7.4 (buffer A). The column was washed with 3-4 column volumes of buffer A to remove the excess PEG linker. Then the product was eluted with a gradient of 0 to 100% 1 M NaCl in 20 mM Tris-HCl buffer, pH 7.4, buffer B in 10 minutes, followed by 100% buffer B for 10 minutes at a flow rate of 10 mL/min. The eluted product was desalted using HiPrep desalting column (50 mL) and lyophilized to solid. Yield 5 mg (oligo equivalent, 50%).

### Example 14

### Compound 17

Boc-ext-amine (1.7 g, 6.4 mmol, 1 eq) was dissolved in 4 mL of DMF. This solution was added to 15 mL of saturated aqueous NaHCO₃ then cooled to 0° C. Maleimide (1 g, 6.4 mmol, 1 eq) was then added and the reaction mixture stirred for 15 minutes followed by addition of 30 mL of water. The reaction continued to stir for 20 minutes at 0° C. The pH was adjusted to 3.5 by addition of H₂SO₄ followed by three extractions with dichloromethane. The combined organic layers were washed once with 0.1 N HCl then once with brine, dried and evaporated under vacuum. The crude product was purified by column chromatography with Ethyl Acetate/Hexane, (8:2, v/v): ¹³C NMR d 28.28, 36.86, 40.19, 67.58, 69.67, 70.00, 78.86, 133.89, 155.63, 170.31.

### Example 15

### Compound 18

To a solution of Boc-ext-maleimide (0.1 g) in 5 mL anhydrous DCM at room temperature was added TFA (2.5 mL). The reaction was monitored by TLC and was determined to be complete after 1.5 hours. The solvents were evaporated under vacuum to give compound 18: ¹³C NMR d 37.26, 39.98, 66.12, 68.36, 69.77, 69.83, 134.11, 160.44, 171.01.

### Example 16

### Compound 19

To a solution of Bsmoc-Gly (0.5 g, 1.7 mmol, 1 eq) and 3,5-dimethyl-4-hydroxy-benzyl-OTBS (0.448 g, 1.7 mol, 1 eq) in 50 mL of anhydrous DCM was added DMAP (0.042 g, 0.34 mol, 5 eq). The mixture was cooled to 0° C. and then EDC (0.408 g, 0.002125 mmol, 0.8 eq) was added. The resulting cloudy solution was warmed to room temperature and stirred overnight. The clear reaction solution was washed with 0.1 N HCl and water. The combined organic layers were dried over MgSO₄, filtered and evaporated under vacuum to give compound 19: ¹³C NMR (CDCl₃-CD₃OD, 1:1, v/v) d 42.10, 56.49, 120.93, 125.21, 129.66, 130.00, 130.18, 133.60, 136.37, 138.63, 155.74, 171.31.

### Example 17

### Compound 20

To a solution of compound 19 (0.089 g, 0.163 mmol, 1.2 eq) in 5 mL of anhydrous DCM was added 4-piperidino-piperidine (0.0247 g, 0.147 mmol, 0.9 eq) at room temperature. The reaction was monitored by TLC and was complete after 4 hours at which 20K 8arm-SCPEG (2.72 g, 0.136 mmol) was added. The reaction was stirred at room temperature overnight. The solvents were partially evaporated under vacuum and the resulting residue was precipitated from ether followed by recrystallization of the solids from DMF/IPA to give compound 20: ¹³C NMR d −5.53, 16.02, 18.05, 25.04, 25.62, 42.01, 63.87, 63.95, 67.31-72.85 (PEG), 125.66, 129.14, 138.36, 146.02, 151.00, 155.96, 167.65, 168.112.

### Example 18

### Compound 21

Compound 20 (1.07 g, 0.05 mmol, 1 eq) and amino-3,6-dioxaoctanoic maleimide (0.60 g, 1.75 mmol, 35 eq) were dissolved in 20 mL DCM, followed by cooling in an ice bath. DIPEA (0.609 mL, 5.5 mmol, 70 eq) was added until a pH of 7-8 was reached. The reaction ran at room temperature for 6.5 hours followed by partial removal of the solvents in vacuo. The solids were then precipitated by ether and flask stored in refrigerator overnight. The solids were then filtered and recrystallized from DMF/IPA to give compound 21: ¹³C NMR d −5.30, 16.28, 18.32, 25.86, 36.90, 40.67, 42.30, 63.72, 64.27, 67.64, 69.23-71.28 (PEG), 125.98, 129.39, 133.92, 138.76, 146.24, 156.08, 167.79, 170.34.

### Example 19

### Compound 22

Compound 21 (0.95 g) was dissolved in 4 mL CH₃CN and 2 mL water followed by addition of mL acetic acid. The mixture was stirred overnight at room temperature followed by removal of the solvents in vacuo. The solids were precipitated with ether and then recrystallized from DMF/IPA to give the alcohol: ¹³C NMR d 15.93, 36.58, 40.35, 41.98, 63.37, 63.60, 67.31, 68.21-70.88 (PEG), 126.46, 129.31, 133.69, 138.67, 146.27, 155.79, 167.58, 170.05. The deprotected benzyl alcohol (1 g, 0.05 mmol, 1 eq) was dissolved in 2 mL DMF and 20 mL DCM followed by cooling the solution to 0° C. DSC (0.1024 g, 0.4 mmol, 8 eq) and pyridine (0.029 ml, 0.36 mmol, 7.2 eq) were added. The reaction mixture gradually warmed to room temperature overnight. The solvents were partially removed in vacuo followed by precipitation of the solids with ether. The crude product was then recrystallized from DMF/IPA: ¹³C NMR d 16.13, 25.24, 36.79, 40.56, 42.21, 63.61, 64.16, 67.54, 69.52-71.29 (PEG), 126.76, 128.56, 130.42, 133.86, 148.01, 155.99, 167.56, 168.20, 170.26.

### Example 20

### Compound 23a-I-R1 (n=8, Oligo I=siRNA, R1=-C-TAT)

Compound 22a (737 mg, 0.0369 mmol) was reacted with siRNA (50 mg, 0.00368 mmol) in 30 mL of pH 7.4 10×PBS buffer. Reaction ran at room temperature for 4 hours. Crude material was purified on Poros with mobile phase A: 20 mmol Tris, pH 7.0 and B: 20 mmol Tris, 2M NaCl, pH 7.0 then desalted with pH 7.0 phosphate buffer. Yield 16.6 mg (oligo eq). Then, 15 mg (oligo eq) of this material was dissolved in 7 mL of pH 7.0 phosphate buffer. SH-TAT (64 mg, 0.039 mmol) was added under nitrogen. The reaction was run for 1.5 hours followed by the purification on Source 15S resin. Column was equilibrated with buffer A (5M urea, 100 mM KH₂PO₄, 25% CH₃CN, pH 6.5). The product was eluted with buffer B (2M KBr). The collected product was desalted on HiPrep desalting column with water and lyophilized. Yield 2.4 mg (oligo eq).

### Example 20A

### Compound 23a-II-R1 (n=8, Oligo II=FITC-Genasense, R1=-C-TAT)

Compound 22a was reacted with FITC-Genasense, followed by reacting with HS—C-TAT in the same reaction conditions described in Example 20 to give the product.

### Example 20B

### Compound 23a-II-R2 (n=8, Oligo II=FITC-Genasense, R1=-C-Arg₉)

Compound 22a was reacted with FITC-Genasense, followed by reacting with HS—C-Arg₉ in the same reaction conditions described in Example 20 to give the product.

### Example 21

### Compound 24

8-Amino-3,6-dioxaoctanoic acid trifluoro acetic acid salt (0.50 g, 0.18 mmol) was dissolved in 12 mL of acetonitrile/water (1/1). The pH of this solution was ˜4. TEA was added to adjust the pH between 8-9. The pH was kept between 8-9. After the addition of Bsmoc-OSu (0.61 g, 0.18 mmol) pH went down to 6. More TEAe was added to bring the pH back to 8-9. The reaction mixture was stirred at room temperature for 45 minutes, and pH remained between 8-9 at the end of the reaction. The reaction mixture was diluted with water (5 mL) and extracted with DCM to remove any remaining starting material. The aqueous layer was acidified with 0.1N HCl and extracted with ethyl acetate. The organic layer was separated and washed with brine, dried over sodium sulfate and evaporated under vacuum to give compound 24 (0.45 g, 65% yield) as a light yellow oil: ¹³C NMR d 173.3, 156.1, 139.5, 137.2, 134.1, 130.8, 130.7, 134.1, 130.8, 130.4, 130.3, 125.8, 121.7, 71.4, 70.4, 70.3, 68.8, 57.1, 41.3, 25.8.

### Example 22

### Compound 25

To a solution of compound 24 (0.41 g, 0.107 mmol) and 3,5-dimethyl-4-hydroxy-benzyl-OTBS (0.283 g, 0.107 mmol) in 40 mL of anhydrous DCM (40 mL) was added DMAP (26 mg, 0.021 mmol). The reaction mixture was cooled to 0° C. and then EDC (0.245 g, 0.128 mmol) was added. The reaction was allowed to warm to room temperature and stirred for 20 hours. The mixture was diluted with water and extracted with DCM, dried over sodium sulfate. The solvent were evaporated under vacuum to give 0.65 g of crude product. Purification on silica gel column, eluting with ethyl acetate/hexane (1:1, v/v) gave compound 25 (0.59 g, 88% yield): ¹³C NMR d 168.2, 155.4, 146.2, 139.5, 138.9, 136.9, 129.4, 126.2, 125.2, 121.3, 70.2, 69.9, 68.1, 64.4, 56.4, 41.1, 26.1, 25.9, 18.5, 16.6, 16.5.

### Example 23

### Compound 26

To a solution of compound 25 (363 mg, 0.6 mmol, 1.2 eq) in 200 mL of anhydrous DCM was added 4-piperidino-piperidine (90.9 mg, 0.54 mmol, 0.9 eq) at room temperature. The reaction was monitored by TLC and was complete after 4 hours at which 20K-8arm-SCPEG (10 g, 0.5 mmol, 1 eq) was added. The reaction was stirred at room temperature overnight. The solvents were partially evaporated under vacuum and the resulting residue was precipitated from ether followed by recrystallization of the solids from DMF/IPA to give compound 26 (9.1 g): ¹³C NMR (75.4 MHz, CDCl₃): d −5.35, 16.28, 18.26, 25.25, 25.80, 40.56, 61.40, 63.66, 64.16, (68.05-73.64, PEG), 125.92, 129.26, 138.64, 145.99, 151.21, 156.01, 167.88, 168.20

### Example 24

### Compound 27

DIEA amine (5.6 mL, 32.2 mmol, 70 eq) was added to a solution of compound 26 (9.2 g, 0.46 mmol, 1 eq) and amino-3,6-dioxaoctanoic maleimide (5.5 g, 16.1 mmol, 35 eq) in 200 mL of anhydrous DCM at 0° C. until a pH of 7-8 was reached. The reaction ran at room temperature for 5 hours followed by partial removal of the solvents under vacuum. The residue was then precipitated by addition of ethyl ether and flask stored in refrigerator overnight. The solids were filtered and recrystallized from DMF/IPA to give compound 27 (7 g): ¹³C NMR d −5.69, 15.90, 17.87, 25.45, 36.44, 40.20, 42.30, 63.19, 64.36, 67.14, 68.05-72.68 (PEG), 125.51, 128.88, 133.57, 138.19, 145.64, 155.64, 167.45, 169.90.

### Example 25

### Compound 28

Compound 27 (7 g) was dissolved in 50 mL acetonitrile and 11 mL water followed by addition of 22 mL acetic acid. The solution was stirred overnight at room temperature followed by removal of solvents under vacuum. The residue was precipitated with ether and then recrystallized from DMF/IPA: ¹³C NMR d 15.97, 36.58, 40.33, 63.35, 63.60, 67.29, 69.08-71.06 (PEG), 126.50, 129.20, 133.68, 138.73, 146.27, 155.76, 167.55, 170.03. The deprotected compound (7 g, 0.35 mmol, 1 eq) was dissolved in 14 mL DMF and 140 mL dichloromethane followed by cooling of the solution to 0° C. DSC (717 mg, 2.8 mmol, 8 eq) and pyridine (0.204 mL, 2.52 mmol, 7.2 eq) were added. The reaction mixture gradually warmed to room temperature overnight. The solvents were partially removed under vacuum followed by precipitation of the solids with ethyl ether. The crude solid was recrystallized from DMF/IPA. ¹³C NMR d 16.13, 22.40, 25.18, 36.73, 40.50, 42.32, 63.54, 67.47, 69.24-71.20 (PEG), 126.70, 128.53, 130.27, 133.81, 148.01, 155.93, 167.55, 168.17, 170.20.

### Example 26

### Compound 29

Compound 28 (1.5 g, 0.075 mmol) was reacted with HIF1-α (20 mg, 0.0036 mmol) in 8 mL of pH 7.8 phosphate buffer. Reaction ran at room temperature for 2 hours. Crude material was purified on Source 15Q resin. Column is equilibrated with buffer A (5M urea, 100 mM KH₂PO₄, 25% CH₃CN, pH 6.5). The product is eluted with buffer B (2M KBr). The collected product was desalted on HiPrep desalting column with water and lyophilized. Product yield 17.7 mg (oligo eq). 8.85 mg (oligo eq) of this product was reacted with 93 mg of HS-TAT in 5 mL of PH 7.0 phosphate buffer. The product was purified on Source 15S resin and desalted. Yield 1.7 mg (oligo eq).

### Example 27

### Compound 30

A solution of 4 N HCl in dioxane (70 mL) was added to BocCys(Npys)-OH (1, 5 g, 13.32 mmol). The suspension was stirred at room temperature for 3 hour, and then was poured into 700 mL of ethyl ether. The solid was filtered through a course fritted funnel without applying vacuum until the end. The cake was washed with ethyl ether (3×50 mL) and then dried under vacuum at room temperature overnight. ¹H NMR (CD₃OD) δ 8.93 (1H, dd, J=1.5, 4.7 Hz), 8.66 (1H, dd, J=1.5, 8.20 Hz), 7.59 (1H, dd, J=4.7, 8.2 Hz), 4.24 (1H, dd, J=4.1, 9.4 Hz), 3.58 (1H, dd, J=4.1, 14.9 Hz), 3.36 (1H, dd, J=9.4, 15.2 Hz). ¹³C NMR (75.4 MHz, CDCl₃): d 169.40, 156.27, 154.64, 144.13, 135.246, 123.10, 52.77, 39.27.

### Example 28

### Compound 31a

To a solution of compound 30 (1.82 g, 5.55 mmol) in DMF/DCM (25 mL/45 mL) was added ^{20K}8arm-PEG-SC (7.30 g, 0.35 mmol). Then, DIEA was added (3 mL, 16.8 mmol) and the resulting suspension was stirred at room temperature for 5 hour. The reaction mixture was evaporated under vacuum and then precipitated with DCM/Et₂O at 0° C. The solid was filtered and then was dissolved in 80 mL of DCM. After addition of 20 mL of 0.1 N HCl, the mixture was stirred for 5 minutes, then transferred to a separatory funnel and the organic layer was separated and washed again with 0.1 N HCl (20 mL) and brine (20 mL). The organic layer was dried over MgSO₄, filtered and evaporated under vacuum. The residue was precipitated with DCM/Et₂O at 0° C. The solid was filtered and dried in the vacuum oven at 30° C. for at least 2 hour: ¹³C NMR d 170.90, 156.66, 155.68, 153.86, 142.41, 133.85, 121.24, 72.96-69.30, 64.08, 53.01, 41.82.

### Example 29

### Compound 31b

To a solution of compound 30 (765 mg, 2.33 mmol) in DMF/DCM (20 mL/40 mL) was added ^{20K}4arm-PEG-SC (6.0 g, 0.29 mmol). Then, DIEA was added (1.2 mL, 6.96 mmol) and the resulting suspension was stirred at room temperature for 5 hours. The reaction mixture was evaporated under vacuum and then precipitated with DCM/Et₂O. The solid was filtered and then was dissolved in 60 mL of DCM. After addition of 15 mL of 0.1 N HCl, the mixture was stirred for 5 min, then transferred to a separatory funnel and the organic layer was separated and washed again with 0.1 N HCl (15 mL) and brine (15 mL). The organic layer was dried over MgSO₄, filtered and evaporated under vacuum. The residue was precipitated with DCM/Et₂O. The solid was filtered and dried in the vacuum oven at 30° C. for at least 2 hours: ¹³C NMR d 170.76, 156.53, 155.57, 153.85, 142.37, 133.79, 121.23, 72.44-69.30, 63.99, 52.95, 45.36, 41.82.

### Example 30

### Compound 32a-I (n=4, Oligo I=LNA Survivin)

To a solution of C6-thio-LNA-survivin (120 mg, 0.021 mmol) in 60 mL pH 6.5 phosphate buffer was added compound 31a (2.3 mg, 0.107 mmol) and the solution was stirred for 1 hour at room temperature. Reaction progress was checked by anion-exchange HPLC. The reaction mixture was filtered through 0.2 micron filter and loaded on Poros anion-exchange column. Product was eluted with a gradient using buffer system 20 mM Tris. HCl 2M NaCl at pH 7.0. Yield after desalting was 80 mg (oligo eq).

### Example 30A

### Compound 32b-I (n=4, Oligo I=LNA Survivin)

To a solution of C6-thio-LNA-survivin (300 mg, 0.054 mmol) in 150 mL pH 6.5 phosphate buffer was added compound 31b (4.8 g, 0.273 mmol) and the solution was stirred for 1 h at room temperature. Reaction progress Was checked by anion-exchange HPLC. The reaction mixture was filtered through 0.2 micron filter and loaded on Poros anion-exchange column. Product was eluted with a gradient using buffer system 20 mM Tris. HCl 2M NaCl at pH 7.0. Yield after desalting was 225 mg (oligo eq).

### Example 31

### Compound 33a-I-R1 (n=8, Oligo I=LNA Survivin, R=R1=-C-TAT)

Compound 32a (80 mg oligo eq, 0.0142 mmol) was dissolved in 20 ml of buffer (5M urea, 100 mM KH₂PO₄). The solution was cooled at 0° C. under nitrogen and then the peptide C-TAT (329 mg, 0.198 mmol) was added. The rich yellow color was observed. Continued to stir the reaction for 1.5 hours under nitrogen atmosphere at 0° C. and then purified by cation-exchange chromatography using the Source 15S resin. Column (10 mm×10 mm) was equilibrated with buffer A (5M urea, 100 mM KH₂PO₄, 25% CH₃CN, pH 6.5) for three column volumes and then the sample was loaded onto the column. The product was eluted with buffer B (2M KBr). The collected product was lyophilized and desalted on HiPrep desalting column with 50 mM pH 7.4 PBS buffer. The desalted solution was then concentrated to about 1 mg/mL (oligo eq) solution. Product yield 21.75 mg (oligo eq).

### Example 31A

### Compound 33a-I-R2 (n=84, Oligo I=LNA Survivin, R=R2=-C-Arg₉)

Compound 32a was reacted with C-Arg₉ in the same reaction conditions described in Example 31 to give the product

### Example 31B

### Compound 33a-I-R3 (n=84, Oligo I=LNA Survivin, R=R3=-C-TAT-RGD)

Compound 32a was reacted with C-TAT-RGD in the same reaction conditions described in Example 31 to give the product.

### Example 31C

### Compound 33b-I-R₁ (n=4, Oligo I, R=R1=-C-TAT)

Compound 32b (20 mg oligo eq, 0.0035 mmol) was dissolved in 10 ml of buffer (5M urea, 100 mM KH₂PO₄). The solution was cooled at 0° C. under nitrogen and then the peptide C-TAT (52 mg, 0.0315 mmol) was added. The rich yellow color was observed. Continued to stir the reaction for 1.5 h under nitrogen at 0° C. and then purified by cation-exchange chromatography using the Source 15S resin. Column (10 mm×10 mm) was equilibrated with buffer A (5M urea, 100 mM KH₂PO₄, 25% CH₃CN, pH 6.5) for three column volumes and then the sample was loaded onto the column. The product was eluted with buffer B (2M KBr). The collected product was lyophilized and desalted on HiPrep desalting column with 50 mM pH 7.4 PBS buffer. The desalted solution was then concentrated to about 1 mg/ml (oligo eq) solution. Product yield 12.5 mg (oligo eq).

### Example 32

### Compound 34

To a solution of 8arm^{20K}SCPEG (1 eq) in DMF is added peptide (16 eq). Then, DIEA is added (32 eq) and the resulting suspension is stirred at room temperature for 5 hours. The reaction mixture is precipitated with DCM/Et₂O at 0° C. The solid is filtered and then is dissolved in water. The crude solid is purified using a C18 reverse-phase chromatography. Product peak is collected and lyophilized to solid.

### Example 33

### Compound 35

Compound 34 is added to a solution of 2% hydrazine in DMF and the solution is stirred for 4 h at room temperature. The reaction mixture is loaded on reverse-phase column and purified. The product peak is collected and lyophilized.

### Example 34

### Compound 36

Compound 35 (1 eq) is dissolved in 20 ml of buffer (5M urea, 100 mM KH₂PO₄). The solution is cooled at 0° C. under nitrogen and then the Oligo-SH (8 eq) is added. Continued to stir the reaction for 1.5 hours under nitrogen at 0° C. and then purified by cation-exchange chromatography using the Source 15S resin. Column (10 mm×10 mm) is equilibrated with buffer A (5M urea, 100 mM KH₂PO₄, 25% CH₃CN, pH 6.5) for three column volumes and then the sample is loaded onto the column. The product is eluted with buffer B (2M KBr). The collected product is lyophilized and desalted on HiPrep desalting column with 50 mM pH 7.4 PBS buffer. The desalted solution is then concentrated to about 1 mg/mL (oligo eq) solution.

### Example 35

### Compound 37

To a solution of 20k-8arm-PEG succinimidyl carbonate (1 eq) in dichloromethane is added H-Cys(StBu)—OH hydrochloride salt (1 eq) and diisopropylethylamine (1 eq). The reaction is stirred at room temperature for about 5 hours. The solvent is partially removed followed by precipitating with ethyl ether. The crude product is collected by filtration and crystallized from 2-propanol.

### Example 36

### Compound 38

Compound 37 (1 eq) and amino-3,6-dioxaoctanoic maleimide (35 eq) are dissolved in dichloromethane followed by cooling of the solution in an ice bath. Diisopropylethyl amine (70 eq) is added until a pH of 7-8 is reached. The reaction runs at room temperature for 6.5 hours followed by partial removal if the solvent in vacuo. Solids are then precipitated by ether and flask stored in refrigerator overnight. Solids are then filtered and recrystallized from DMF/IPA.

### Example 37

### Compound 39

Compound 38 (215 mg, 0.011 mmol, 1 eq) is dissolved in buffer (5M urea, 100 mM KH₂PO₄). The solution is cooled at 0° C. under nitrogen and then SH-TAT (250 mg, 14 eq) is then added. Continue to stir the reaction for 1.5 hours under nitrogen at 0° C. followed by the purification on Source 15S resin. Column is equilibrated with buffer A (5M urea, 100 mM KH₂PO₄, 25% CH₃CN, pH 6.5). The product is eluted with buffer B (2M KBr). The collected product is lyophilized and desalted on HiPrep desalting column with 50 mM PBS (pH 7.4). The desalted solution is then concentrated to about 1 mg/mL solution.

### Example 38

### Compound 40

To a solution of Compound 39 (1 eq) in water is added dithiothreitol (2 eq). The reaction is stirred at room temperature for two hours and then solvent is removed. The crude material is crystallized from isopropanol and then mixed with Oligo-S—S-Py (3 eq) in 100 mM phosphate buffer, pH 6.5 at room temperature for 2 hours. The reaction is purified on Source 15S resin. Column is equilibrated with buffer A (5M urea, 100 mM KH₂PO₄, 25% CH₃CN, pH 6.5). The product is eluted with buffer B (2M KBr). The collected product was lyophilized and desalted on HiPrep desalting column with 50 mM PBS (pH 7.4). The desalted solution is then concentrated to about 1 mg/mL solution.

### Example 39

### Compound 41

20k 8armPEG-OH (2.0 g, 0.1 mmol) was dissolved in DCM (20 mL). TEA (1.62 g, 16.0 mmol) was added. This solution was added to acryloyl chloride (0.724 g) in DCM (10 mL) at 0° C. over 1 hour. The reaction mixture was stirred at 0° C. overnight. This solution was added to IPA/ether (250 mL/250 ml) at 0° C. The solids formed were filtered. The wet solids were dissolved in DCM and washed with 0.4 N HCl. The organic layer was dried with magnesium sulfate and filtered through celite. Solvent was removed and residue was recrystallized from DCM/ether. ¹³C NMR (75.4 MHz, CDCl₃): δ 165.5, 130.5, 127.8, 71.0-67.1 (PEG), 63.2.

### Example 40

### Compound 42

To a solution of C6-thio-LNA-survivin (100 mg, 0.018 mmol) in 60 mL pH 8.0 phosphate buffer was added compound 41 (3.6 g, 0.18 mmol) and the solution was stirred for 1 hour at room temperature. Reaction progress was checked by anion-exchange HPLC. The reaction mixture was filtered through 0.2 micron filter and loaded on Poros anion-exchange column. Product was eluted with a gradient using buffer system 20 mM Tris. HCl 2M NaCl at pH 7.0. Yield after desalting was 60 mg (oligo eq).

### Example 41

### Compound 43

Compound 42 (8 mg, 0.0014 mmol, oligo eq) was mixed with SH-TAT-RGD (111 mg, 0.0496 mmol) in 3 mL of buffer (5M urea, 100 mM KH₂PO₄) under nitrogen. The reaction was run for 2 hours. The crude product was purified on Source 15S resin. Column is equilibrated with buffer A (5M urea, 100 mM KH₂PO₄, 25% CH₃CN, pH 6.5). The product is eluted with buffer B (2M KBr). The collected product was desalted on HiPrep desalting column, lyophilized and yield 57 μg.

### Example 42

### Compound 46

To 1,2-di(pyridin-2-yl)disulfane (Compound 44) (8.8 g, 39.9 mmol) in 50 mL anhydrous ethyl acetate was added 3-mercaptopropanoic acid (4.2 g, 39.9 mmol) in dark followed by 13 drops of trifluoro borane etherate. The reaction was stirred for 5 hours in dark and then filtered. Then 50 mL of cold ethyl acetate was added to the solids. The filtrate was then rotovaped to about 50 mL solution of compound 45. To this solution t-butyl carbazate (4.8 g, 36.3 mmol) was added followed by DCC (7.5 g, 36.3 mmol). The reaction was stirred for 16 hours at room temperature in dark and then it was filtered, evaporated and purified by column chromatography using 1:1 mixture of hexanes/ethyl acetate to give 8.1 g of product Compound 46. ¹³CNMR d 170.1, 158.9, 155.2, 149.5, 137.0, 121.1, 120.4, 81.5, 34.9, 33.7, 28.2.

### Example 43

### Compound 47

To tert-butyl 2-(3-(pyridin-2-yldisulfanyl)propanoyl)hydrazinecarboxylate (Compound 46) (8.1 g, 24.6 mmol) in 64 mL DCM was added 16 mL TFA at 0° C. The reaction was stirred at rt for 1 hour. After completion of reaction the solvent was rotovaped and then the residue was precipitated from 20/300 mL of DCM/Et₂O at 0° C. Solids were filtered and dried to get 5.5 g of compound 47: ¹³C NMR d 173.8, 159.6, 147.9, 138.5, 121.1, 120.7, 33.4, 32.2.

### Example 44

### Compound 49

To 3,3-diethoxypropan-1-amine (Compound 48) (5.2 g, 35.3 mmol) in 30 mL DCM was added Fmoc-OSu (24 g, 70.6 mmol) at 0° C. and then warmed to rt. The reaction was stirred for 2 hours at room temperature until no starting material was observed by TLC. The reaction was then diluted with 30 mL DI water. The aqueous layer was extracted with 2×30 mL DCM and then the organic layer was dried over anhydrous magnesium sulfate, filtered and concentrated. The crude material was purified by column chromatography using DCM as the eluting solvent to get 5.7 g of compound 49: ¹³C NMR d 156.2, 143.9, 141.2, 127.5, 126.9, 125.0, 119.8, 102.2, 66.5, 61.8, 47.3, 37.2, 33.3, 15.4.

### Example 45

### Compound 50

Compound 49 (200 mg) was stirred in 86% formic acid (1.1 mL) for 1 hour at room temperature. The solvent was removed with Rotavapor at room temperature under vacuum and the residue was dissolved in DCM (30 mL). The solution was washed with water (30 mL). The organic layer separated was dried with magnesium sulfate. The solvent was removed completely to give white solids compound 50 (145 mg): ¹³C NMR d 156.2, 143.7, 141.2, 127.6, 126.9, 124.9, 119.9, 66.7, 47.2, 44.1, 34.5.

### Example 46

### Compound 51

Compound 47 (258.3 mg, 0.8746 mmol) and compound 50 (300 mg, 0.8746 mmol) were dissolved in THF (15 mL). Molecular sieves were added. The reaction was completely in 10 minutes. The molecular sieves were filtered after reaction. Solvent was removed and residue was washed with ethyl ether to give crude compound 51 (385 mg).

### Example 47

### Compound 52

Without further purification, compound 51 (270 mg, 0.53 mmol) was treated with 10% (w/v) DMAP. (0.54 g) in DMF (5.4 mL) under nitrogen at room temperature for 8.5 hours to give compound 52. 20k 8armSCPEG (650 mg, 0.033 mmol) was added in situ to the reaction mixture. The reaction was left at RT overnight. Solvent was removed and residue was precipitated with DCM/ether. The wet solids isolated were recrystallized from acetonitrile/IPA twice to give compound 10 (630 mg) with E & Z isomers: ¹³C NMR d 172.1, 166.8, 159.7, 159.1, 156.0, 149.1, 149.0, 144.8, 136.9, 136.7, 120.7, 120.3, 119.7, 119.3, 78.0-69.2 (PEG), 63.5, 37.6, 37.5, 34.4, 34.1, 33.2, 32.8, 32.4, 32.1.

### Example 48

### Compound 54

To a solution of C6-thio-LNA-survivin (10 mg, 0.0018 mmol) in 5 mL pH 7.0 phosphate buffet was added compound 53 (0.36 g, 0.018 mmol) and the solution was stirred for 1 hour at room temperature. Reaction progress was checked by anion-exchange HPLC. The reaction mixture was filtered through 0.2 micron filter and loaded on Poros anion-exchange column. Product was eluted with a gradient using buffer system 20 mM Tris. HCl 2M NaCl at pH 7.0. Yield after desalting was 2 mg (oligo eq).

### Example 49

### Compound 55

Compound 54 (3 mg, 0.00053 mmol, oligo eq) was mixed with SH-TAT-RGD (16.7 mg, 0.00743 mmol) in 1 mL of pH 7.0 phosphate buffer under nitrogen. The reaction was run for 2 hours. The crude product was purified on Source 15S resin. Column is equilibrated with buffer A (5M urea, 100 mM KH₂PO₄, 25% CH₃CN, pH 6.5). The product is eluted with buffer B (2M KBr). The collected product was desalted on HiPrep desalting column and lyophilized.

### Example 50

### Compound 56

To a solution of 20K4ArmPEGNHS (5 g, 0.25 mmol) in 50 mL of anhydrous DCM was added 4-aminopropionaldehyde diethylacetal (0.04 g, 0.275 mmol) at room temperature. The reaction mixture was stirred at room temperature for 20 hours. The solvents were evaporated under vacuum and the crude compound was crystallized with acetonitrile/IPA to give compound 56 as a white solid (4.7 g): ¹³C NMR d 168.17, 155.87, 151.38, 101.37, 70.21, 69.89, 63.46, 61.29, 45.22, 36.78, 33.24, 25.19, 15.12.

### Example 51

### Compound 57

To a solution of compound 30 (0.36 g, 0.117 mmol) in 10 mL of anhydrous DCM was added DIPEA (0.40 mL, 0.233 mmoles) at room temperature. To the stirred mixture a solution of compound 56 (4.00 g, 0.0194 mmol) in 30 mL of anhydrous DCM was added, followed by DMF (13 mL). The reaction mixture was stirred at rt for 5 hours. The solvents were evaporated under vacuum and the resulting residue was precipitated with DCM/ethyl ethyl. The crude compound was recrystallized with acetonitrile/IPA to give compound 57 as a white solid (3.6 g): ¹³C NMR d 170.74, 156.53, 155.49, 153.75, 142.27, 133.69, 121.08, 101.44, 70.66, 69.70, 69.17, 63.89, 63.51, 62.71, 61.34, 53.37, 52.91, 45.27, 41.74, 36.84, 33.27, 25.15, 15.15.

### Example 52

### Compound 58

To a solution of compound 57 (0.70 g, 0.034 mmol) in chloroform was added (85%) formic acid (0.15 mL) at room temperature. Reaction mixture was stirred at room temperature for 20 hours. The solvents were evaporated under vacuum. The crude oil was triturated with ether to give compound 58 as a light yellow solid (0.65 g): ¹³C NMR: d 170.72, 161.87, 160.59, 156.59, 55.57, 153.76, 142.33, 133.75, 121.14, 70.30, 69.75, 69.19, 68.59, 63.98, 63.75, 62.77, 61.40, 53.55, 52.93, 45.31, 43.88, 41.76, 34.26.

### Example 53

### Compound 59

Compound 58 (53 mg, 0.026 mmol) was reacted with C10-survivin hydrazide (6 mg, 0.885 μmol) in 2 mL of pH 7.0 phosphate buffer. Reaction ran at room temperature for 2 hours. Crude material was purified on Poros with mobile phase A 20 mmol Tris, pH 7.0 and B: 20 mmol Tris, 2M NaCl, pH 7.0 then desalted with water. Yield 1.5 mg (oligo eq). 1.2 mg (oligo eq) of this material was dissolved in 0.5 mL of buffer (5M urea, 100 mM KH₂PO₄). SH-TAT (2.3 mg, 0.00138 mmol) was added under nitrogen. The reaction was run for 1.5 hours followed by the purification on Source 15S resin. Column is equilibrated with buffer A (5M urea, 100 mM KH₂PO₄, 25% CH₃CN, pH 6.5). The product is eluted with buffer B (2M KBr). The collected product was desalted on HiPrep desalting column with pH7.4 PBS and lyophilized. Yield 250 μg (oligo eq).

### Example 54

### Compound 60

To a solution of 4-Hydroxy-3,5-dimethyl benzaldehyde (1.36 g, 10 mmol) in anhydrous methanol (5 mL) is added 1.0 M Lithium tetrafluoro borate (0.3 mL) followed by trimethyl orthoformate (1.378 g, 13 mmol). The reaction mixture is refluxed for 3 hours and then quenched by addition of saturated sodium bicarbonate (20 mL). The mixture is extracted with ethyl acetate twice (60 mL, 30 mL). The combined organic layers are washed with saturated sodium chloride (20 mL) and dried over MgSO₄. After filtration the solvents are evaporated under vacuum to give compound 60.

### Example 55

### Compound 62

To a solution of compound 61 (10 g, 0.25 mmol) in anhydrous DCM (100 mL) is added compound 60 (50.0 mg, 0.275 mmol) followed by DMAP (33.6 mg, 0.275 mmol). The mixture is refluxed overnight. The solvents are evaporated under vacuum and the residue is crystallized with DCM/ether. The wet solids are isolated and recrystallized from CNCH₃/IPA to give compound 62.

### Example 56

### Compound 63

To a solution of compound 62 (10 g, 0.25 mmol) in anhydrous DCM (100 mL) is added compound 18 (342 mg, 1.5 mmol) followed by DMAP (183 mg, 1.5 mmol). The mixture is refluxed overnight. The solvents are evaporated under vacuum and the residue is crystallized with DCM/ether. The wet solids are isolated and recrystallized from CNCH₃/IPA to give compound 63.

### Example 57

### Compound 64

Compound 63 (0.7 g, 0.0175 mmol) is dissolved in chloroform (0.6 mL). Formic acid (85%, 0.15 mL) is added. The mixture is stirred overnight. The solvents are evaporated under vacuum and the residue is recrystallized from DCM/ether to give compound 64.

### Example 58

### Compound 65

Compound 64 is mixed with SH-TAT-RGD in pH 7.0 phosphate buffer under nitrogen. The reaction is run for 2 hours. The crude product is purified on Source 15S resin. Column is equilibrated with buffer A (5M urea, 100 mM KH₂PO₄, 25% CH₃CN, pH 6.5). The product is eluted with buffer B (2M KBr). The collected product is desalted on HiPrep desalting column and lyophilized.

### Example 59

### Compound 68

To a solution of 8armPEG-SC (5.5 g, 0.26 mmol) in 115 mL of anhydrous DCM was added compound 66 (117.2 mg, 0.28 mmol, 1.1 eq). The reaction mixture was stirred overnight and then, compound 67 (1.75 g, 4.52 mmol, 17.5 eq) in 60 mL of THF was added and the mixture stirred at room temperature for 4 days. The solvents were remove under vacuum and the resulting solid was recrystallized twice with IPA to give compound 68 (4.4 g): ¹³C NMR d 27.93, 35.19, 37.27, 52.53, 52.87, 53.03, 56.26, 56.64, 61.09, 62.77, 63.60, 69.35-70.51 (PEG), 126.67, 127.78, 128.73, 137.38, 155.87, 169.47.

### Example 60

### Compound 69

Compound 68 was added to a TFA/DCM solvent mixture (50/100 mL) and the mixture was stirred at room temperature overnight. The solvents were removed under vacuum and the residue was precipitated by addition of ethyl ether. The solids were filtered and recrystallized with IPA to give the carboxylic acid of compound 3 (4.6 g): ¹³C NMR d 33.88, 35.54, 48.65, 49.72, 50.68, 51.48, 56.47, 56.85, 59.67, 61.05, 64.18, 69.05-70.36 (PEG), 128.79, 129.81, 156.43, 169.30. To a 0° C. solution of the carboxylic acid (3.3 g, 0.14 mmol, 1 eq) and 3,5-dimethyl-4-hydroxy-benzyl-OTBS (114 mg, 0.43 mmol, 3 eq) in 52 mL of anhydrous DCM were added DMAP (105 mg, 0.86 mmol, 6 eq) and EDC (110 mg, 0.57 mmol, 4 eq). The reaction mixture was stirred at room temperature. The solvents were removed under vacuum and the residue was precipitated with DCM/ethyl ether. The resulting solids were filtered and recrystallized with IPA to give compound 69 (3 g): ¹³C NMR d −5.29, 16.35, 25.86, 34.15, 36.34, 50.01, 51.36, 52.12, 56.67, 56.93, 60.54, 61.52, 63.92; 64.21, 69.25-71.34 (PEG), 125.98, 127.88, 128.12, 128.36, 129.23, 156.17, 169.90.

### Example 61

### Compound 70

Compound 69 (3 g) was dissolved in 12 mL acetonitrile and 6 mL water followed by addition of 30 mL acetic acid. Solution stirred overnight at room temperature followed by removal of solvents in vacuo. Solids were precipitated with ether and then recrystallized from DMF/IPA to give deprotected alcohol. Alcohol (2.7 g, 0.08 mmol, 1 eq) was dissolved in 3 mL DMF and mL dichloromethane followed by cooling of the solution to 0° C. DSC (170 mg, 0.65 mmol, 8 eq) then pyridine (46 μL, 0.57 mmol, 7.2 eq) were added. Reaction mixture gradually warmed to room temperature overnight. Partially removed DCM in vacuo followed by precipitation of the solids with ether. Solids were then recrystallized from DMF/IPA to give compound 70 (2.3 g).

### Example 62

### Compound 71

To a solution of oligo-NH₂ (3 mg, 0.5 μmol) in PBS buffer (1.5 mL, pH 7.8) was added Compound 70 (140 mg, 5 μmol) and stirred at room temperature for 2 hours. The reaction mixture was diluted to 10 mL with water and loaded on a Poros HQ, strong anion exchange column (10 mm×1.5 mm, bed volume ˜16 mL) which was pre-equilibrated with 20 mM Tris-HCl buffer, pH 7.0 (buffer A). The column was washed with 3-4 column volumes of buffer A to remove the excess PEG linker. Then the product was eluted with a gradient of 0 to 100% 1 M NaCl in 20 mM Tris-HCl buffer, pH 7.0, buffer B in 10 minutes, followed by 100% buffer B for 10 minutes at a flow rate of 10 mL/min. The eluted product was desalted using HiPrep desalting column (50 mL) and lyophilized to give compound 71. Yield 2.2 mg (oligo equivalent, 73%).

### Biological Data

### Example 63

### In Vitro Cellular Uptake for Compound 23a-II-R1

The cellular uptake by cancer cells was measured to determine the effect of conjugation of oligonucleotides to PEG polymer including the positively-charged moieties. The inventive conjugate (23a-II-R1) contains seven arms attached to C-TAT (SEQ ID NO: 1) and one arm attached to 5′ antisense BCL-2 oligonucleotide, TCTCCCAGCGTGCGCCAT, (SEQ ID NO: 6). The control conjugate is similar to compound 23a-II-R1, but does not contain the positively charged moiety TAT. Both oligonucleotides of compound 101 and control oligonucleotides were labeled with FITC by methods provided by the supplier.

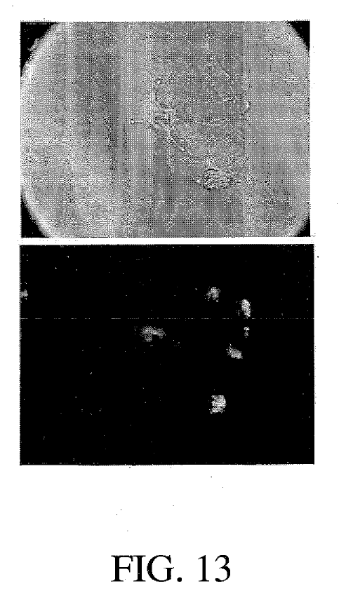

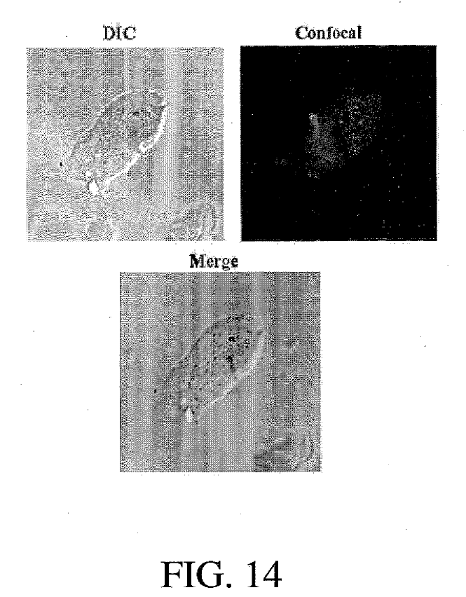

A549 human lung cancer cells with 10% FBS growth medium in a 4 well plate were incubated over night at 37° C. Cells were transfected with each of the test compounds, washed three times with PBS, and added 50% glycerol in PBS (20 ml 100% glycerol+20 ml PBS) to cover the cells on slides. The slides were stored at 4° C. over night. Fluorescent microscopy and confocal microscopy were used to show cellular uptake of PEG-oligonucleotides. Cellular uptake of the test compounds is shown in FIG. 13 (fluorescent microscope image) and FIG. 14 (confocal microscope image).

The data shows that cancer cells uptake the negatively charged therapeutic agents such as oligonucleotides conjugated to the positively-charged polymers. The data indicates that the positive charge backbone of the polymers allows the therapeutic oligonucleotides to cross the cell membrane and reach to the target site in the tumor cells.

### Example 64

### Efficiency of Cellular Uptake of 23a-II-R1

Compound 23a-II-R1 was used to show cellular uptake efficiency of the compound with or without transfection agents. A549 human lung cancer cells in the medium containing 10% FBS growth medium in a 6 well plate were incubated over night at 37° C. Thereafter, the medium was removed and cells were treated with 1 ml/well 10% FBS growth medium containing each of the test compounds. Control compound is an oligonucleotide, antisense BCL-2 oligonucletide (SEQ ID: 7), not conjugated to the polymer or the positively charged moiety. Both control and inventive compounds were labeled with FITC to show cellular uptake of the compounds.

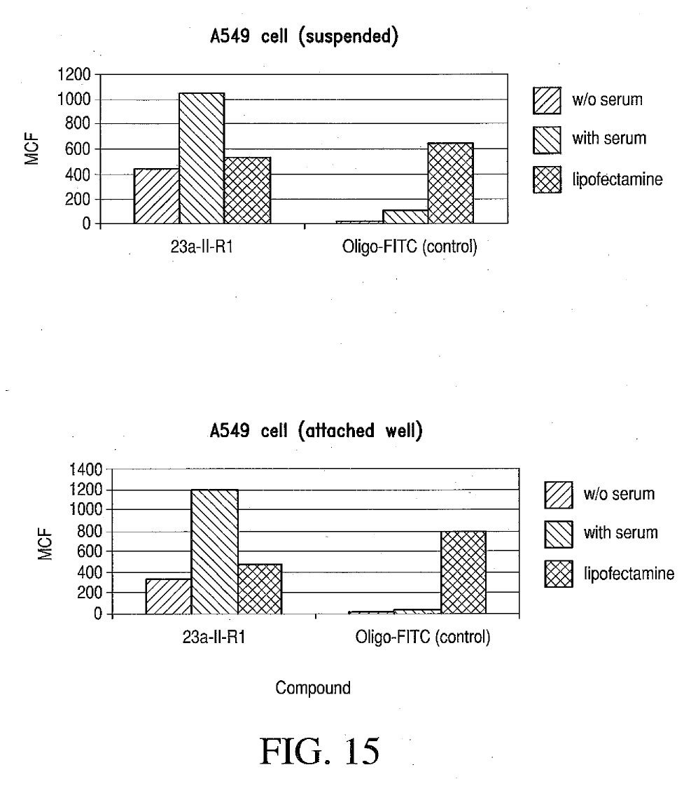

The results are set forth in FIG. 15. The oligonucleotides attached to compound 23a-II-R1 were taken by the cells more than the control oligonucleotides without transfection agents. The cellular uptake of oligonucleotides conjugated to the positively charged polymer was significantly improved when the medium contained serum, which is similar to the environment in vivo, compared to the naïve oligonucleotide.

The results indicate that the inventive polymers increase delivery of the negatively charged therapeutic agents such as oligonucleotides into the target cells and thus the therapy based on oligonucleotides can benefit from this advantage.

### Example 65

### Dose Dependent Cellular Uptake of 23a-II-R1 and 23a-II-R2

Flow cytometry was used to show cellular uptake efficiency of the oligonucleotides conjugated to positively charged polymers. A549 human lung cancer cells in the medium containing 10% FBS growth medium in a 6 well plate were incubated over night at 37° C. Thereafter, the medium was removed and cells were treated with 1 ml/well 10% FBS growth medium containing each of compound 23a-II-R1 and native oligonucleotides (SEQ ID NO:6). After the treatment, cells were harvested, trypsinized, washed with 1% BSA PBS three times and analyzed using FACS. The oligonucleotide of compound 101 and the control oligonucleotides were labeled with FITC.

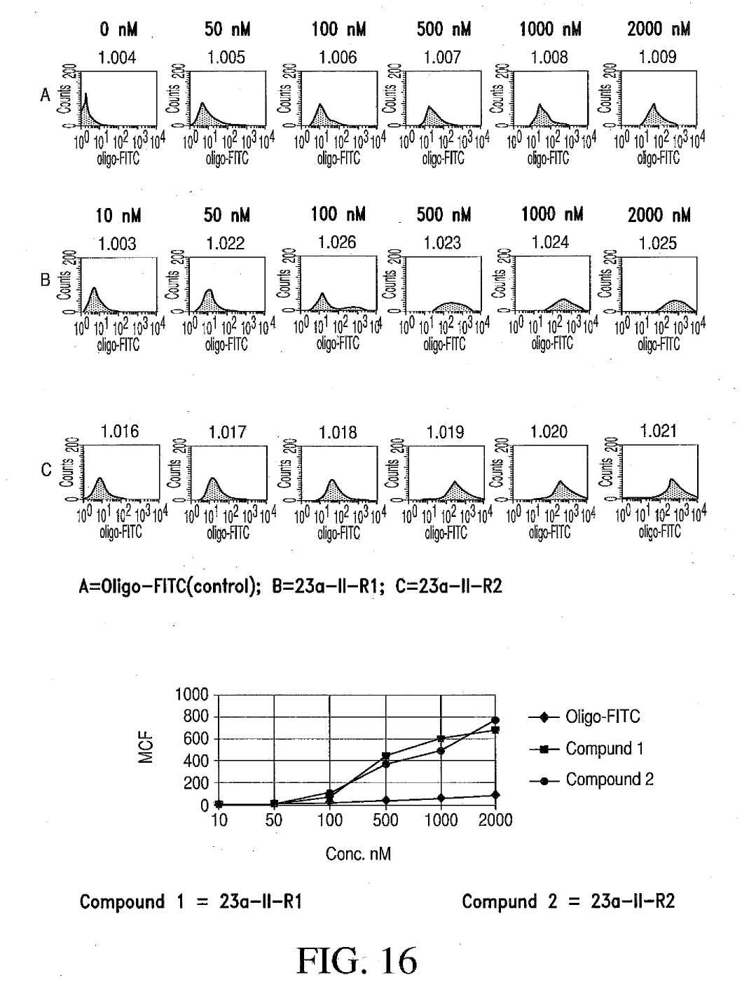

The results were shown in FIG. 16. The results show that the oligonucleotides conjugated to the positively charged polymer containing either TAT (23a-II-R1) or Arg₉ (23a-II-R2) were uptaken by cells in a dose-dependent manner. This property can be advantages in treatment of cancers because clinicians adjust dosage of therapeutic oligonucleotides depending on the need of patients.

### Example 66

### BCL2 mRNA Downregulation of 23a-I-R1

This study was conducted to determine whether the oligonucleotides uptaken by cancer cells downregulate specific gene expression involved in cancer. A431 cells were transfected with native oligonucleotides and compound 23a-I-R1 without transfection agent. The positively charged polymer conjugate contains TAT and BCL2 siRNA.

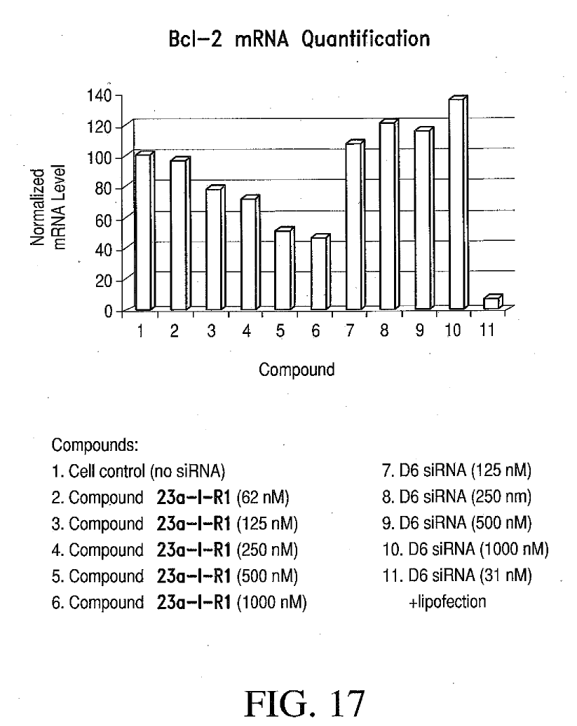

The RT-PCR analysis of BCL2 mRNA is set forth in FIG. 17. These results show that both oligonucleoties of compound 102 and control downregulated BCL2 mRNA expression dose-dependently in human lung cancer cells. The BCL2 siRNA conjugate to the positively charged polymers showed significantly higher down regulation of BCl2 mRNA expression compared to native Bcl2 siRNA.

The results indicate that show that PEG-oligonucleotide conjugates including antisense oligonucleotides or siRNA described herein allow use of siRNA as therapeutics.

### Example 67

### Survivin mRNA Downregulation by [RGD-TATC-S—S]₇-^{20K}8arm PEG-S—S-Antisense Survivin LNA in A549 Cell Model (Solid Tumor, Lung Cancer)

This study was conducted to determine the effects of the positively charged polymers on Survivin mRNA expression. A549 human lung cells were transfected with each of compounds 33b-I-R4, 33b-I-R5 and 33a-I-R3 in concentrations of 1000 nM, 200 nM, 40 nM, 8 nM and 1.6 nM. Both compounds 33b-I-R4 ([linear RGD-S—S]₃-^{20K}4arm PEG-S—S-antisense Survivin LNA) and 33b-I-R5 ([cyclic RGD-S—S]₃-^{20K}4arm PEG-S—S-antisense Survivin LNA) contain the antisense Survivin LNA but do not include the positively charged peptide (TAT). Compound 33a-I-R3 ([RGD-TATC-S—S]₇-^{20K}8arm PEG-SS-antisense Survivin LNA) includes the TAT peptide and antisense Survivin LNA. The Survivin mRNA expression in the A549 cells treated with each of the compounds was measured by RT-PCR one day after the treatment.

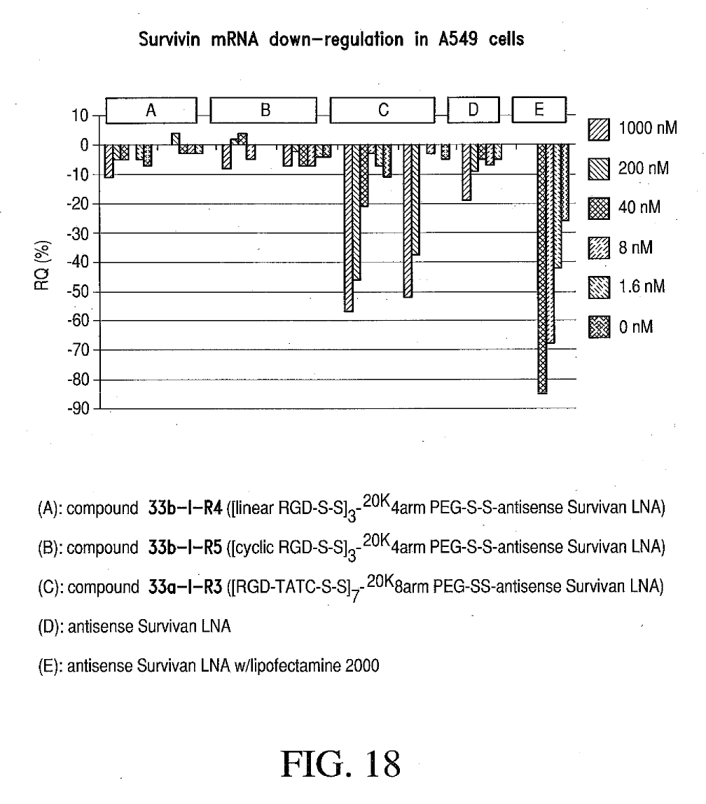

The compound including the TAT peptide significantly downregulated Survivin mRNA expression without the transfection agent. The downregulation was dose-dependent. These results are shown in FIG. 18. Neither the antisense Survivin LNA of the compounds without the TAT peptide nor the native antisense Survivin LNA inhibited Survivin mRNA expression. The data shows that the positively charged polymers are beneficial to treatment utilizing negatively charged oligonucleotides.

### Example 68

### Survivin mRNA Downregulation by [RGD-TATC-S—S]₇-^{20K}8arm PEGS-S-Antisense Survivin LNA in DU145 Cell Model (Solid Tumor, Prostate Cancer)

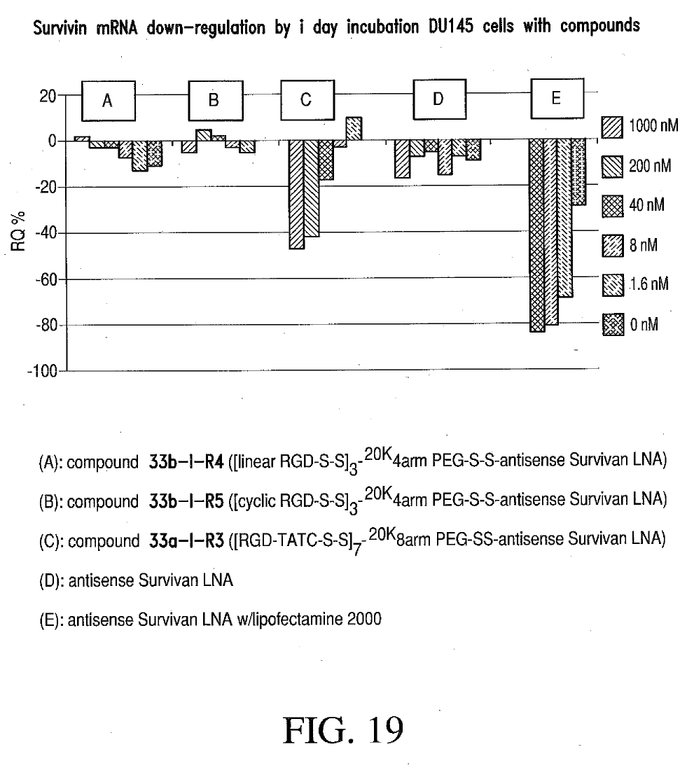

DU145 cells were transfected with the same compounds used in Example 67. As in Example 67, the compound containing the TAT peptide showed significant down-regulation of Survivin mRNA expression. Neither the native antisense Survivin LNA nor the antisense Survivin LNA of the compounds without the positively charged peptide down-regulated Survivin mRNA expression in the DU145 cells. These results are shown in FIG. 19. The data indicates that the positively charged polymers can be beneficial to treatment of various types of cancers. The Survivin mRNA downregulation was similarly observed with the study with compound 33a-I-R1 (TATC-S—S)₇-^{20K}8arm PEG-S—S-antisense Survivin LNA) in DU145 cells.

### Example 69

### Survivin mRNA Downregulation by [(Arg)₉C—S—S]₇-^{20K}8arm PEG-S—S-Antisense Survivin LNA in A549 Cell Model

A549 human lung cancer cells were transfected with each of compound 33a-I-R2 and the naive antisense Survivin LNA. Compound 33a-I-R2 ([(Arg)₉C—S—S]₇-^{20K}8arm PEG-S—S-antisense Survivin LNA) includes seven polymer arm terminals connected to C(Arg)₉ and one arm terminal connected to the antisense Survivin LNA via the intracellular releasable disulfide bond. The naive oligonucleotides (antisense Survivin LNA) were also transfected with the transfection agent lipofectamine.

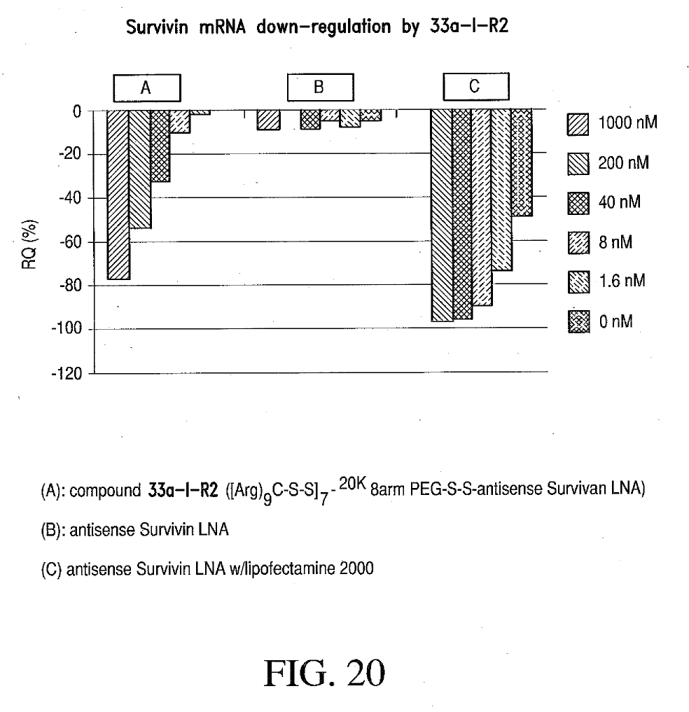

The compound including the (Arg)₉ significantly downregulated Survivin mRNA expression without the transfection agent. The results are shown in FIG. 20. The data indicates that the inventive polymers containing the positively charged peptide such as TAT and (Arg)₉ allow therapeutic oligonucleotides to be delivered into a target site inside the cells. The oligonucleotide-based anticancer therapy can benefit from the positively charged polymers.

### Example 70

### Survivin mRNA Downregulation by Positively Charged Polymers Containing Intracellular Labile Linkers

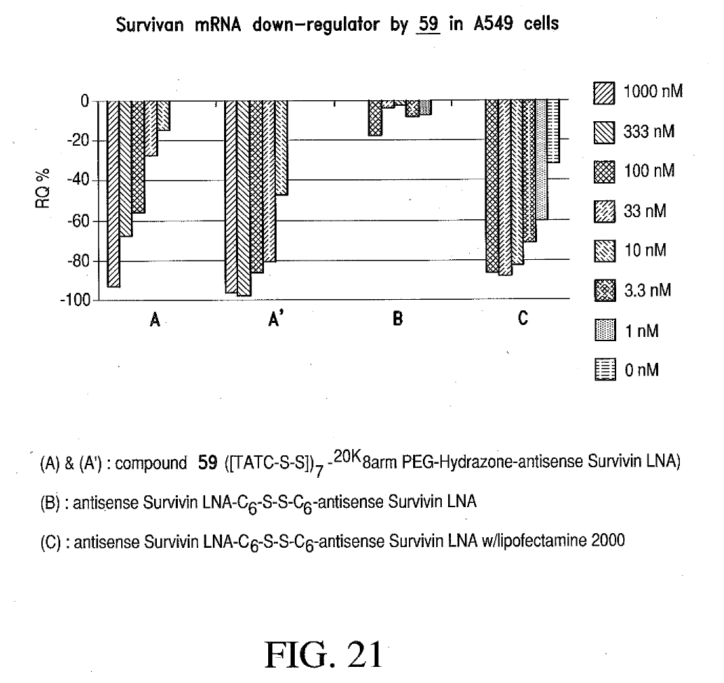

A549 cells were transfected with each of compound 59 and the antisense Survivin LNA dimer. The dimer of the antisense Survivin LNA modified with a C₆—SH tail (antisense Survivin LNA-C₆—S—S—C₆-antisense Survivin LNA) was also transfected with the transfection agent. Compound 59 contains a hydrazone-based releasable linker. The mRNA downregulation results are shown in FIG. 21.

The antisense Survivin LNA attached to the polymers via the hydrazone linker downregulated Survivin mRNA expression. The data indicates that the antisense oligonucleotides connected via the hydrazone linker can be released from the polymers inside the cells after crossing the cell membrane. It indicates that the polymers can employ various types of releasable linkers such as disulfide bond and hydrazone-based linkers and modify release rate and site of the antisense oligonucleotides from the polymers.

### Example 71

### Survivin mRNA Downregulation by [RGD-TATC-S—S]₇-^{20K}8arm PEGS-S-Antisense Survivin LNA in A549 Cell Model

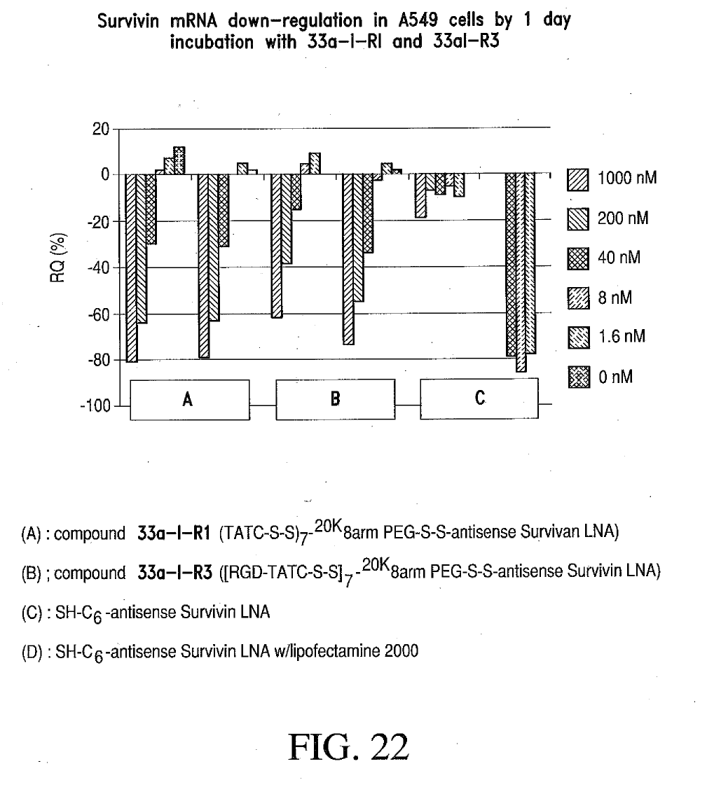

This study was conducted to determine whether the positively charged polymers containing a targeting agent is as effective as the positively charged polymers without a targeting agent and thus the polymers containing the targeting agent can be utilized for targeted delivery. A549 cells were transfected with each of compounds 33a-I-R1 (TATC-S—S)₇-^{20K}8arm PEG-S—S-antisense Survivin LNA) and 33a-I-R3 ([RGD-TATC-S—S]₇-^{20K}8arm PEG-S—S-antisense Survivin LNA). In compounds 33a-I-R1 and 33a-I-R3, seven polymer arm terminals are connected to C-TAT and C-TAT-RGD, respectively. The cells were also transfected with the antisense Survivin LNA modified with a SH—C₆ tail with or without the transfection agent. Both polymers with or without the targeting agent downregulated Survivin mRNA expression. The results are shown in FIG. 22. This feature of the positively charged polymers is beneficial to target agent directed delivery of oligonucleotide therapeutics.

### Example 71

### Specific Inhibition of Survivin mRNA Expression

This study was conducted to determine whether the oligonucleotides selectively inhibit gene expression after crossing the cancer cell membrane.

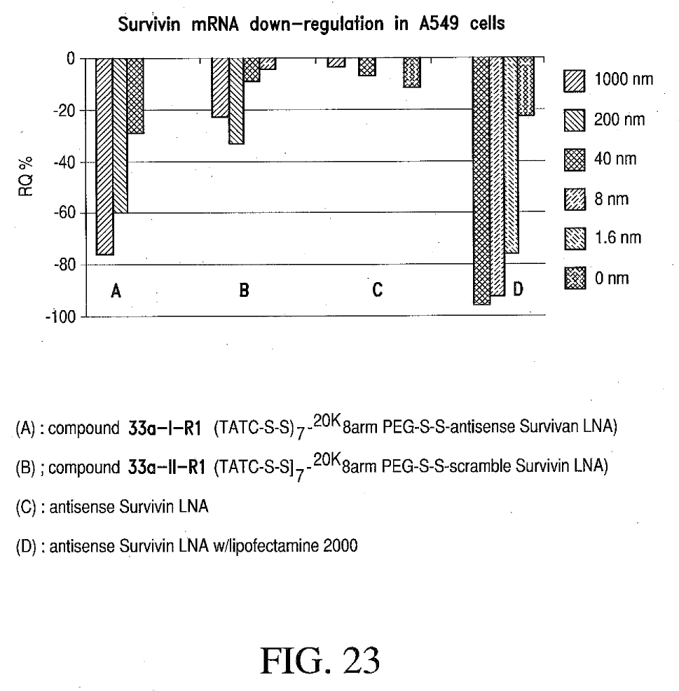

A549 human lung cancer cells were transfected with each of compound 33a-I-R1 (TATC-S—S)₇-^{20K}8arm PEG-S—S-antisense Survivin LNA), compound 33a-II-R1 (TATC-S—S)₇-^{20K}8arm PEG-S—S-scrambled Survivin LNA) and the native antisense Survivin LNA. Compound 33a-II-R1 corresponds to compound 33a-II-R1 except in that it includes mismatching nucleotides within the antisense Survivin LNA (scrambled Survivin LNA: 5′-s^{m}CₛGₛ^{m}CₛAₛgₛaₛtₛtₛaₛgₛaₛaₛAₛ^{m}Cₛ^{m}Cₛt-3′). The naive antisense Survivin LNA was also transfected with the transfection agent. The results are shown in FIG. 23.

The results show that the antisense Survivin LNA of compound 33a-I-R1 significantly inhibited Survivin mRNA expression compared to the mismatching antisense Survivin LNA of compound 33a-II-R1 and the naïve antisense Survivin LNA. The antisense Survivin LNA containing mismatching nucleotides did not inhibit Survivin gene expression. The mRNA down-regulation is specific inhibition. This feature is desirable to have unwanted gene expression to be selectively downregulated in treatment of cancer.

### Example 73

### In Vivo Survivin Downregulation in Calu-6 Tumor

Survivin downregulation efficacies of three analogs of PEG containing antisense Survivin LNA were evaluated in mice xenographed with Calu 6 tumor cells. Each group was treated with compound 33a-I-R1 (TATC-S—S)₇-^{20K}8arm PEG-S—S-antisense Survivin LNA), compound 33a-I-R3 ([RGD-TATC-S—S]₇-^{20K}8arm PEG-SS-antisense Survivin LNA) or compound 33a-I-R2 ([(Arg)₉C—S—S]₇-^{20K}8arm PEG-S—S-antisense Survivin LNA).

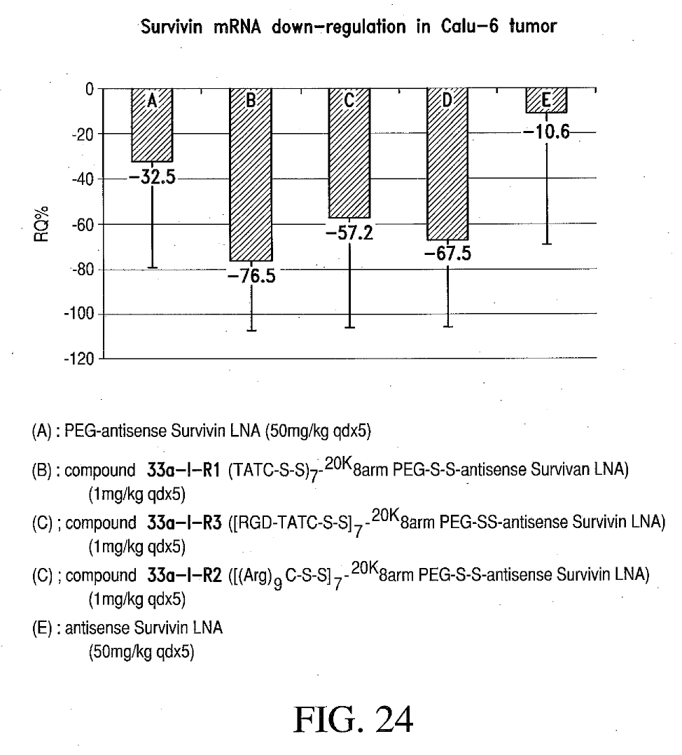

After treatment, tumor tissues were excised when the mice were sacrificed and Survivin mRNA expression was measured. All three polymers including antisense Survivin LNA significantly inhibited Survivin mRNA expression in tumor tissues compared to naive antisense Survivin LNA. The results are set forth in FIG. 24. The results show that the oligonucleotides connected to the positively charged polymers are significantly more effective than native antisense Survivin LNA in the treatment of cancer such as solid tumor.

1. A compound of the formula (I):
{Z₂}_{b}—R₁-{Z₁}ₐ
wherein
each Z₁ is independently 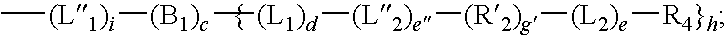
each Z₂ is independently selected capping groups, 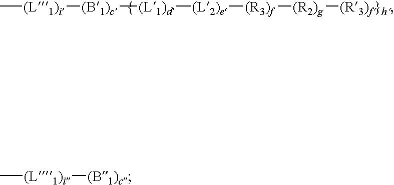
R₁ is a substantially non-antigenic polymer;
R₂ and R′₂ are independently selected positive charge-containing peptides or nitrogen-containing cyclohydrocarbon moieties;
R₃ and R′₃ are independently selected targeting agents;
R₄ is a biologically active moiety;
B₁, B′₁ and B″₁ are independently selected branching groups;
L₁, L′₁, L₁″, L₁′″ and L₁″″ are independently selected bifunctional linkers;
L₂, L′₂ and L″₂ are independently selected releaseable linkers;
(a) is a positive integer;
(b) is zero or a positive integer;
(c), (c′) and (c″) are independently zero or a positive integer;
(d), (d′), (i), (i′) and (ii) are independently zero or a positive integer;
(e) is a positive integer;
(e′) and (e″) are independently zero or a positive integer;
(f) and (f′) are independently zero or a positive integer;
(g) is a positive integer;
(g′) is zero or a positive integer; and
(h) and (h′) are independently a positive integer;
provided that (g′) is a positive integer when (b) is not zero and all Z₂ are capping groups, -(L″″₁)_{i″}-(B″₁)_{c″} or in combination.

2. The compound of claim 1, having the formula:

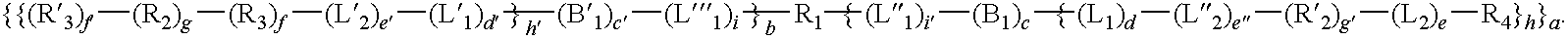

3. The compound of claim 1, wherein the sum of (a) and (b) is from about 1 to about 32.

4. The compound of claim 1, wherein the sum of (a) and (b) is 2, 3, 4, 8, 16, or 32.

5. The compound of claim 1, wherein Z₂ is a capping group and (a) and (g′) are 1.

6. The compound of claim 1, wherein (a) is 1 and (b) is a positive integer from 1 to 7.

7. The compound of claim 1, wherein the biologically active moiety is selected from the group consisting of —NH₂ containing moieties, —OH containing moieties and —SH containing moieties.

8. The compound of claim 1, wherein the biologically active moiety is selected from the group consisting of pharmaceutically active compounds, enzymes, proteins, oligonucleotides, antibodies, monoclonal antibodies, single chain antibodies and peptides.

9. The compound of claim 1, wherein the biologically active moiety comprises an oligonucleotide

10. The compound of claim 9, wherein the oligonucleotide is selected from the group consisting of antisense oligonucleotides, locked nucleic acids (LNA), short interfering RNA (siRNA), microRNA (miRNA), aptamers, peptide nucleic acid (PNA), phosphorodiamidate morpholino oligonucleotides (PMO), tricyclo-DNA, double stranded oligonucleotide (decoy ODN), catalytic RNA (RNAi), aptamers, spiegelmers, CpG oligomers and in combination.

11. The compound of claim 1, wherein the biologically active moiety is selected from the group consisting of antisense Bcl-2 oligonucleotides, antisense HIF-1a oligonucleotides, and antisense Survivin oligonucleotides.

12. The compound of claim 1, wherein the peptide contains from about 1 to about 50 positively charged amino acids.

13. The compound of claim 1, wherein the peptide contains from about 2 to about positively charged amino acids.

14. The compound of claim 1, wherein the peptide comprises CYGRKKRRQRRR (SEQ ID NO: 1) or CRRRRRRRRR (SEQ ID NO: 2).

15. The compound of claim 1, wherein the nitrogen-containing cyclohydrocarbon has the formula: 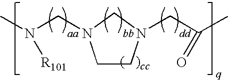
wherein
(aa) is a positive integer from about 2 to 10;
(bb) is 1, 2 or 3;
(cc) is 1 or 2;
(dd) is a positive integer from about 1 to about 5;
R₁₀₁ is independently selected from the group consisting of hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, substituted alkanoyloxy and arylcarbonyloxy; and
(q) is an positive integer from about 2 to about 30.

16. The compound of claim 15, wherein the nitrogen-containing cyclohydrocarbon is selected from the group consisting of:

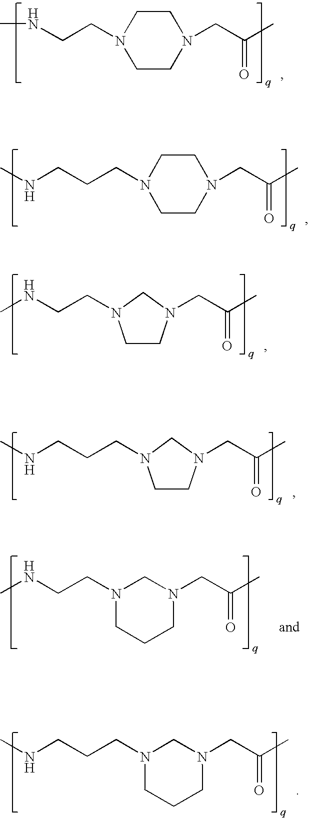

17. The compound of claim 1, wherein the targeting agent is selected from the group consisting of monoclonal antibodies, single chain antibodies, cell adhesion peptides, cell penetrating peptides, receptor ligands, targeting carbohydrate molecules or lectins and oligonucleotide.

18. The compound of claim 1, wherein the targeting agent is selected from the group consisting of RGD peptide, selectin, TAT, penetratin, (Arg)₉ and folic acid.

19. The compound of claim 1, wherein B₁ and B′₁ are independently selected from the group consisting of: 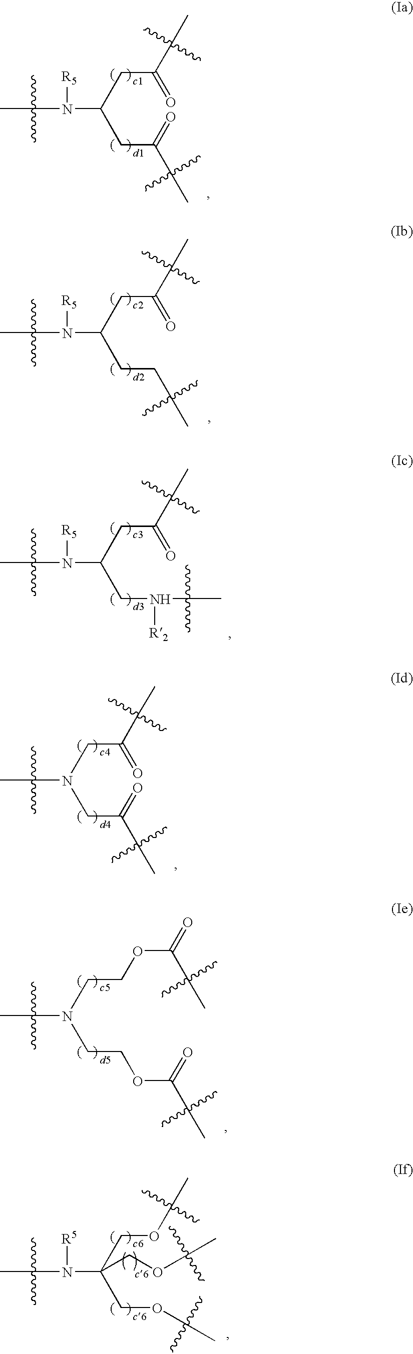 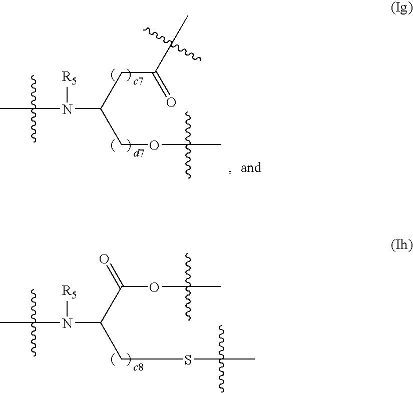
wherein
R₅ is independently selected from the group consisting of hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, and substituted arylcarbonyloxy;
(c1), (c2), (c3), (c4), (c5), (c6), (c′6), (c″6), (c7) and (c8) are independently zero or a positive integer, and
(d1), (d2), (d3), (d4), (d5) and (d7) are independently zero or a positive integer.

20. The compound of claim 19, wherein B₁ and B′₁ are independently selected from the group consisting of:

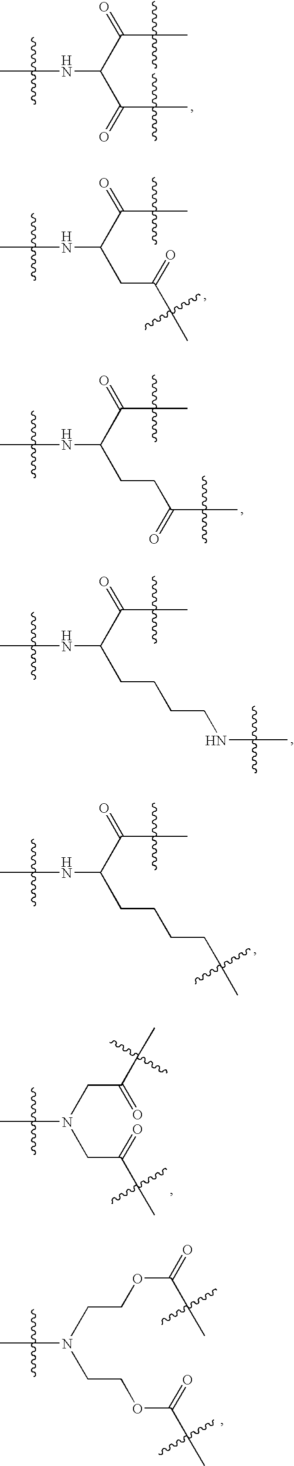

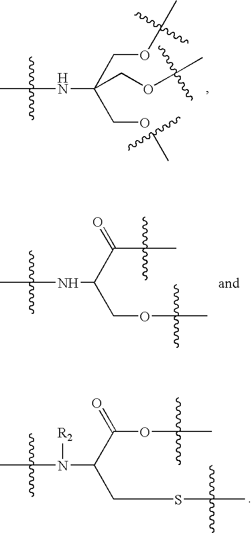

21. The compound of claim 1, wherein L₁ and L′₁ are independently selected from the group consisting of an amino acid and an amino acid derivative.

22. The compound of claim 1, wherein L₁ and L′₁ are independently selected from the group consisting of:
—[C(═O)]ᵥ(CR₂₂R₂₃)ₜ[C(═O)]_{v′}—,
—[C(═O)]ᵥ(CR₂₂R₂₃)ₜ—O[C(═O)]_{v′}—,
—[C(═O)]ᵥ(CR₂₂R₂₃)ₜ—NR₂₆[C(═O)]_{v′}—,
—[C(═O)]ᵥO(CR₂₂R₂₃)ₜ[C(═O)]_{v′}—,
—[C(═O)]ᵥO(CR₂₂R₂₃)ₜO[C(═O)]_{v′}—,
—[C(═O)]ᵥO(CR₂₂R₂₃)ₜNR₂₆[C(═O)]_{v′}—,
—[C(═O)]ᵥNR₂₁(CR₂₂R₂₃)ₜ[C(═O)]_{v′}—,
—[C(═O)]ᵥNR₂₁(CR₂₂R₂₃)ₜO[C(═O)]_{v′}—,
—[C(═O)]ᵥNR₂₁(CR₂₂R₂₃)ₜNR₂₆[C(═O)]_{v′}—,
—[C(═O)]ᵥ(CR₂₂R₂₃)ₜO—(CR₂₈R₂₉)_{t′}[C(═O)]_{v′}—,
—[C(═O)]ᵥ(CR₂₂R₂₃)ₜNR₂₆—(CR₂₈R₂₉)_{t′}[C(═O)]_{v′}—,
—[C(═O)]ᵥ(CR₂₂R₂₃)ₜS—(CR₂₈R₂₉)_{t′}[C(═O)]_{v′}—,
—[C(═O)]ᵥO(CR₂₂R₂₃)ₜO—(CR₂₈R₂₉)_{t′}[C(═O)]_{v′}—,
—[C(═O)]ᵥO(CR₂₂R₂₃)ₜNR₂₆—(CR₂₈R₂₉)_{t′}[C(═O)]_{v′}—,
—[C(═O)]ᵥO(CR₂₂R₂₃)ₜS—(CR₂₈R₂₉)_{t′}[C(═O)]_{v′}—,
—[C(═O)]ᵥNR₂₁(CR₂₂R₂₃)ₜO—(CR₂₈R₂₉)_{t′}[C(═O)]_{v′}—,
—[C(═O)]ᵥNR₂₁(CR₂₂R₂₃)ₜNR₂₆—(CR₂₈R₂₉)_{t′}[C(═O)]_{v′}—,
—[C(═O)]ᵥNR₂₁(CR₂₂R₂₃)ₜS—(CR₂₈R₂₉)_{t′}[C(═O)]_{v′}—,
—[C(═O)]ᵥ(CR₂₂R₂₃CR₂₈R₂₉O)ₜNR₂₆[C(═O)]_{v′}—,
—[C(═O)]ᵥ(CR₂₂R₂₃CR₂₈R₂₉O)ₜ[C(═O)]_{v′}—,
—[C(═O)]ᵥO(CR₂₂R₂₃CR₂₈R₂₉O)ₜNR₂₆[C(═O)]_{v′}—,
—[C(═O)]ᵥO(CR₂₂R₂₃CR₂₈R₂₉O)ₜ[C(═O)]ᵥ—,
—[C(═O)]ᵥNR₂₁(CR₂₂R₂₃CR₂₈R₂₉O)ₜNR₂₆[C(═O)]_{v′}—,
—[C(═O)]ᵥNR₂₁(CR₂₂R₂₃CR₂₈R₂₉O)ₜ[C(═O)]_{v′}—,
—[C(═O)]ᵥ(CR₂₂R₂₃CR₂₈R₂₉O)ₜ(CR₂₄R₂₅)_{t′}[C(═O)]_{v′}—,
—[C(═O)]ᵥO(CR₂₂R₂₃CR₂₈R₂₉O)ₜ(CR₂₄R₂₅)_{t′}[C(═O)]_{v′}—,
—[C(═O)]ᵥNR₂₁(CR₂₂R₂₃CR₂₈R₂₉O)ₜ(CR₂₄R₂₅)_{t′}[C(═O)]_{v′}—,
—[C(═O)]ᵥ(CR₂₂R₂₃CR₂₈R₂₉O)ₜ(CR₂₄R₂₅)_{t′}O[C(═O)]_{v′}—,
—[C(═O)]ᵥ(CR₂₂R₂₃)ₜ(CR₂₄R₂₅CR₂₈R₂₉O)_{t′}[C(═O)]_{v′}—,
—[C(═O)]ᵥ(CR₂₂R₂₃)ₜ(CR₂₄R₂₅CR₂₈R₂₉O)_{t′}NR₂₆[C(═O)]_{v′}—,
—[C(═O)]ᵥO(CR₂₂R₂₃CR₂₈R₂₉O)ₜ(CR₂₄R₂₅)_{t′}O[C(═O)]_{v′}—,
—[C(═O)]ᵥO(CR₂₂R₂₃)ₜ(CR₂₄R₂₅CR₂₈R₂₉O)_{t′}[C(═O)]_{v′}—,
—[C(═O)]ᵥO(CR₂₂R₂₃)ₜ(CR₂₄CR₂₅CR₂₈R₂₉O)_{t′}NR₂₆[C(═O)]_{v′}—,
—[C(═O)]ᵥNR₂₁(CR₂₂R₂₃CR₂₈R₂₉O)ₜ(CR₂₄R₂₅)_{t′}O[C(═O)]_{v′}—,
—[C(═O)]ᵥNR₂₁(CR₂₂R₂₃)ₜ(CR₂₄R₂₅CR₂₈R₂₉O)_{t′}[C(═O)]_{v′}—,
—[C(═O)]ᵥNR₂₁(CR₂₂R₂₃)ₜ(CR₂₄R₂₅CR₂₈R₂₉O)_{t′}NR₂₆[C(═O)]_{v′}—, 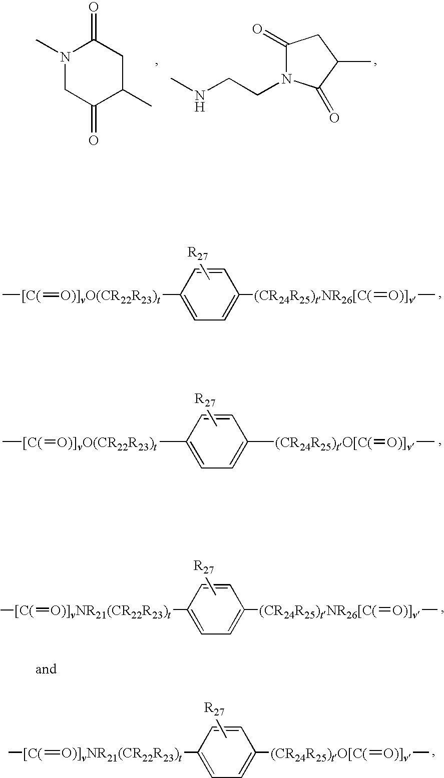
wherein:
R₂₁₋₂₉ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
(t) and (t′) are independently zero or a positive integer; and
(v) and (v′) are independently zero or 1.

23. The compound of claim 1, wherein L₁ and L′₁ are independently selected from the group consisting of:

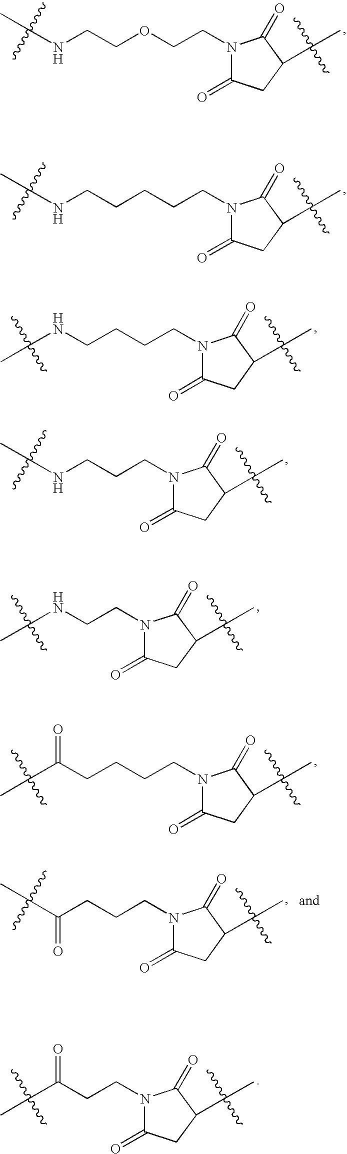

24. The compound of claim 1, wherein L₂ and L′₂ are independently selected from the group consisting of benzyl elimination-based linkers, trialkyl lock-based linkers, bicine-based linkers, acid labile linkers, lysosomally cleavable peptides and cathepsin B cleavable peptides.

25. The compound of claim 24, wherein the acid labile linker is selected from the group consisting of a disulfide, a hydrazone-containing linker and a thiopropionate-containing linker.

26. The compound of claim 24, wherein L₂ and L′₂ is independently selected from the group consisting of: 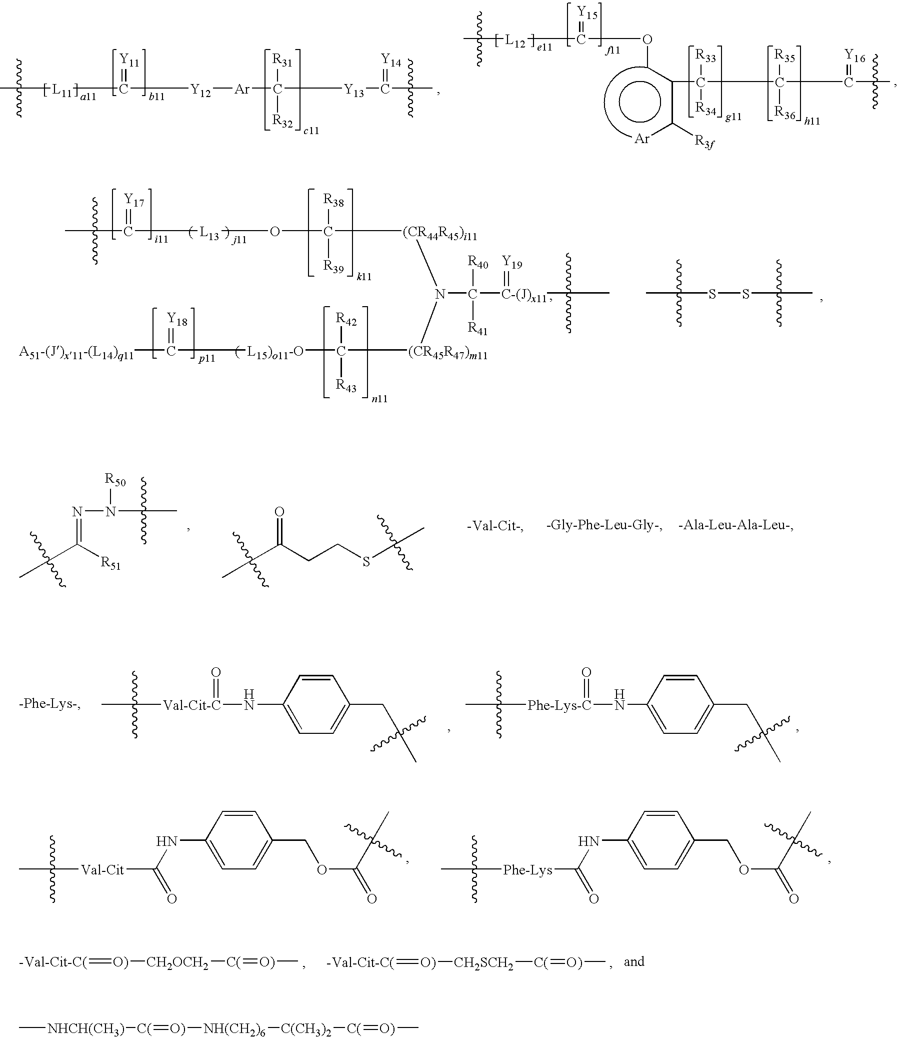
wherein,
Y₁₁₋₁₉ are independently O, S or NR₄₈;
R₃₁₋₄₈, R₅₀₋₅₁ and A₅₁ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
Ar is an aryl or heteroaryl moiety;
L₁₁₋₁₅ are independently selected bifunctional spacers;
J and J′ are independently selected from selected from among moieties actively transported into a target cell, hydrophobic moieties, bifunctional linking moieties and combinations thereof;
(c11), (h11), (k11), (l11), (m11) and (n11) are independently selected positive integers;
(a11), (e11), (g11), (j11), (o11) and (q11) are independently either zero or a positive integer; and
(b11), (x11), (x′11), (f11), (i11) and (p11) are independently zero or one.

27. The compound of claim 1, wherein the capping group is selected from the group consisting of H, NH₂, OH, CO₂H, C₁₋₆ alkoxy and C₁₋₆ alkyl.

28. The compound of claim 1, wherein R₁ comprises a linear, branched or multi-armed polyalkylene oxide.

29. The compound of claim 28, wherein the polyalkylene oxide is selected from the group consisting of a linear, branched or multi-armed polyethylene glycol and a linear, branched or multi-armed polypropylene glycol.

30. The compound of claim 28, wherein the polyalkylene oxide is selected from the group consisting of:
—Y₇₁—(CH₂CH₂O)ₙ—CH₂CH₂Y₇₁—,
—Y₇₁—(CH₂CH₂O)—CH₂C(═Y₂₂)—Y₇₁—,
—Y₇₁—C(═Y₇₂)—(CH₂)ₐ₂—Y₇₃—(CH₂CH₂O)ₙ—CH₂CH₂—Y₇₃—(CH₂)ₐ₂—C(═Y₇₂)—Y₇₁— and
—Y₇₁—(CR₇₁R₇₂)ₐ₂—Y₇₃—(CH₂)_{b2}—O—(CH₂CH₂O)ₙ—(CH₂)_{b2}—Y₇₃—(CR₇₁R₇₂)ₐ₂—Y₇₁—,
wherein:
Y₇₁ and Y₇₃ are independently O, S, SO, SO₂, NR₇₃ or a bond;
Y₇₂ is O, S, or NR₇₄;
R₇₁₋₇₃ are independently selected from the group consisting of hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy and substituted arylcarbonyloxy;
(a2) and (b2) are independently zero or a positive integer; and
(n) is an integer from about 10 to about 2300.

31. The compound of claim 28, wherein the polyalkylene oxide comprises a polyethylene glycol of the formula, —O—(CH₂ CH₂O)ₙ—
wherein (n) is an integer from about 10 to about 2,300.

32. The compound of claim 1, wherein R₁ has an average molecular weight from about 2,000 to about 100,000 daltons.

33. The compound of claim 1, wherein R₁ has an average molecular weight of from about 5,000 to about 60,000 daltons.

34. The compound of claim 1, wherein R₁ has an average molecular weight from about 5,000 to about 25,000 daltons or from about 20,000 to about 45,000 daltons.

35. A compound of claim 1 having a formula selected from the group consisting of: 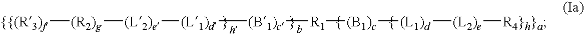
wherein
(e) is 1 or 2;
(e′) is 0, 1 or 2; and
(f) is 0 or 1; and 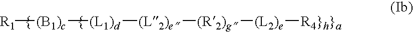
wherein (g′) is a positive integer.

36. The compound of claim 1 having the formula: 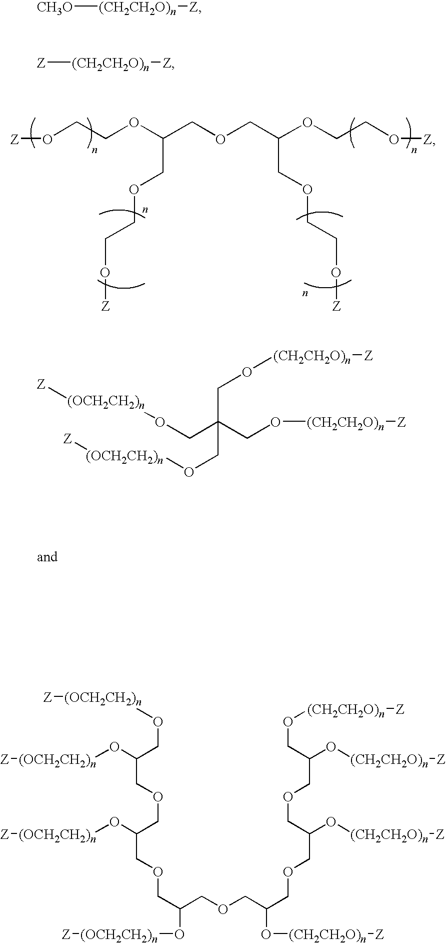
wherein
each Z is Z₁ or Z₂
wherein
each Z₁ is independently 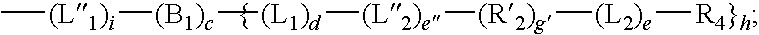
each Z₂ is independently selected capping groups, 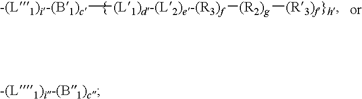
L₂, L′₂ and L″₂ are independently releasable linkers selected from the group consisting of a disulfide, hydrazone-containing linkers, thiopropionate-containing linkers, benzyl elimination-based linkers, trialkyl lock-based linkers and bicine-based linkers, lysosomally cleavable peptides and cathepsin B cleavable peptides;
(c), (c′) and (c″) are independently zero or a positive integer;
(d), (d′), (i), (i′) and (i″) are independently zero or a positive integer;
(e) is a positive integer;
(e′) and (e″) are independently are zero or a positive integer;
(f) and (f′) are independently zero or a positive integer;
(g) is a positive integer;
(g′) is zero or a positive integer;
(h) and (h′) are independently a positive integer, and
all other variables are previously defined,
provided that (g′) is a positive integer when all Z₂ are capping groups, -(L″″₁)_{i″}-(B″₁)_{c″} or in combination.

37. A compound of claim 1 selected from the group consisting of:

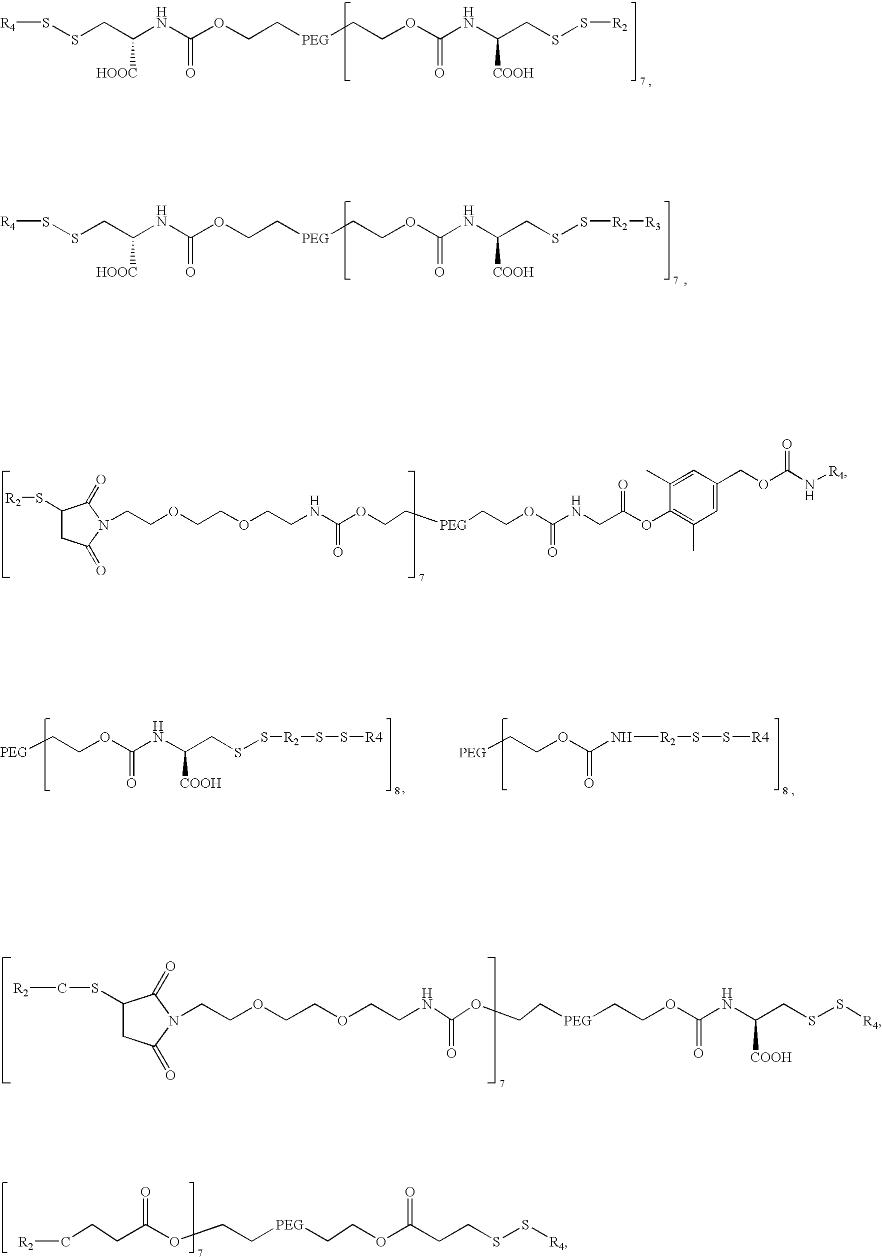

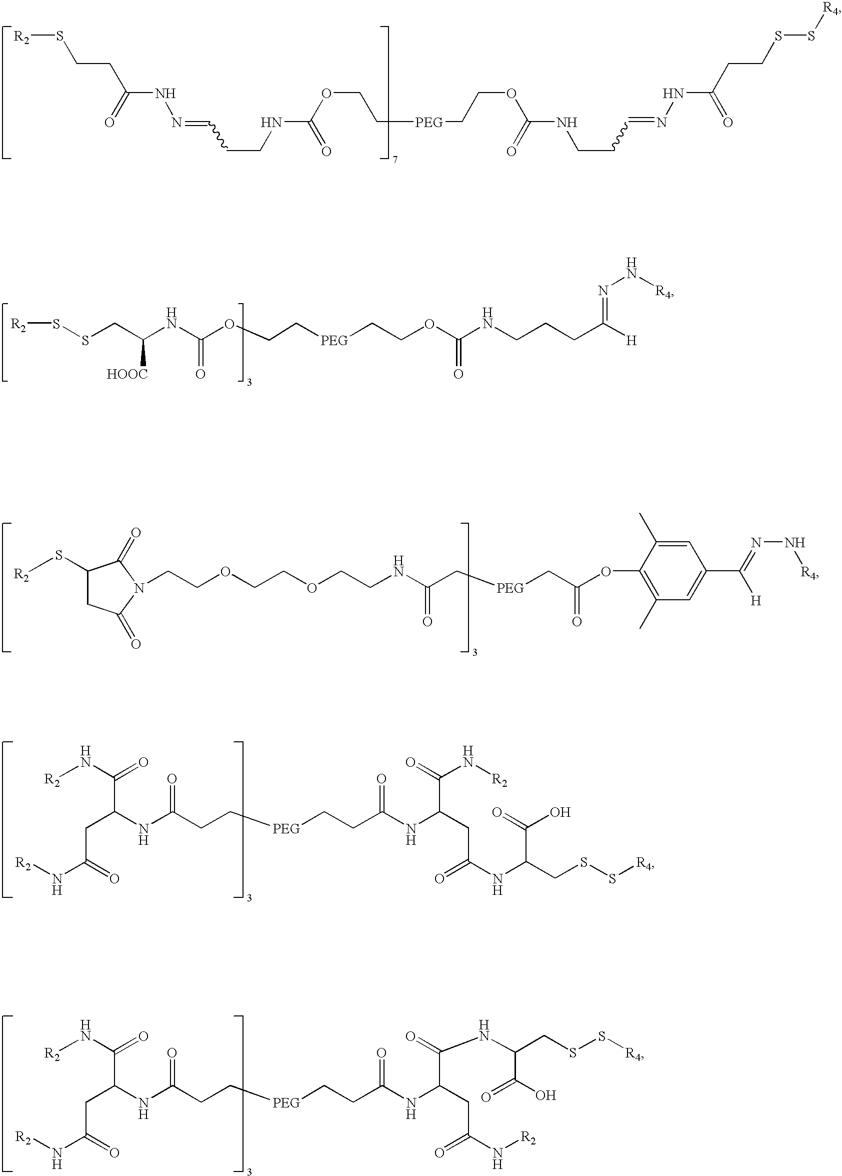

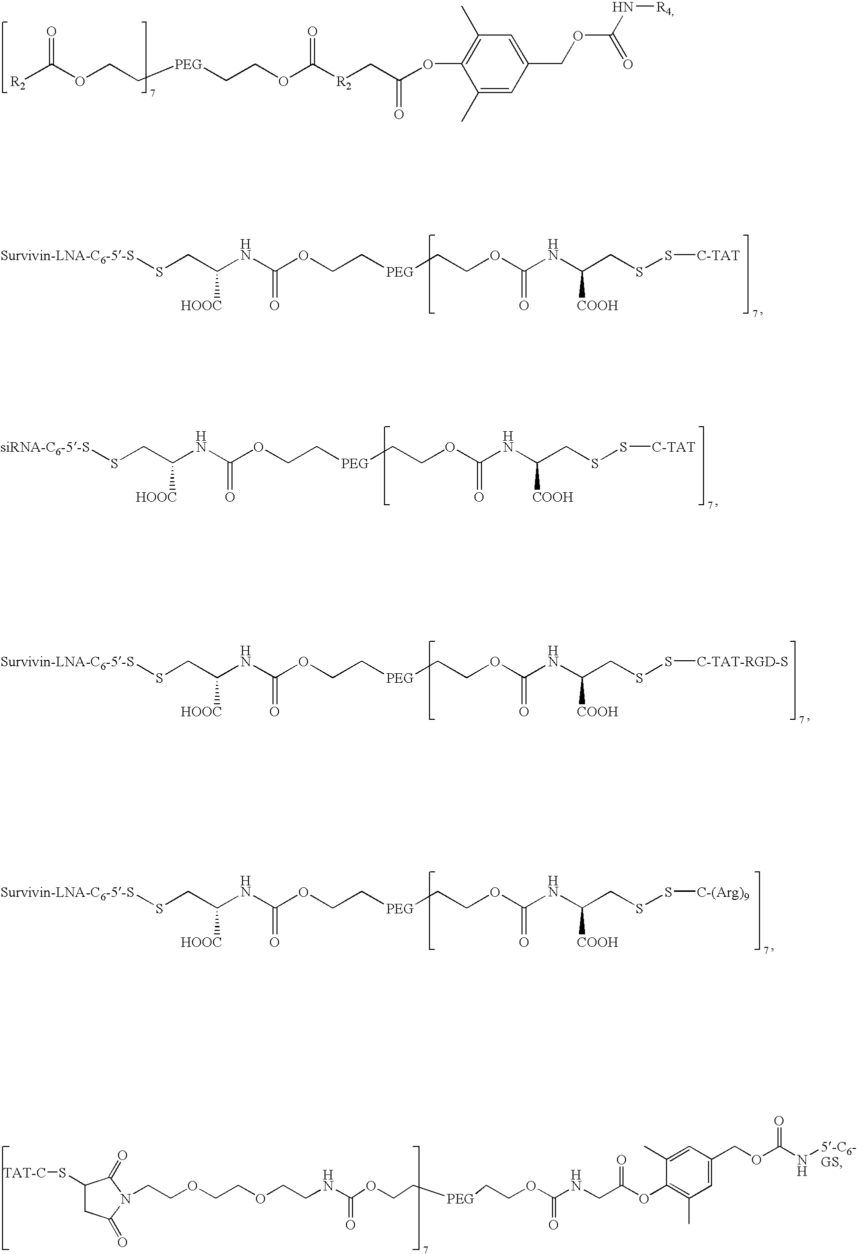

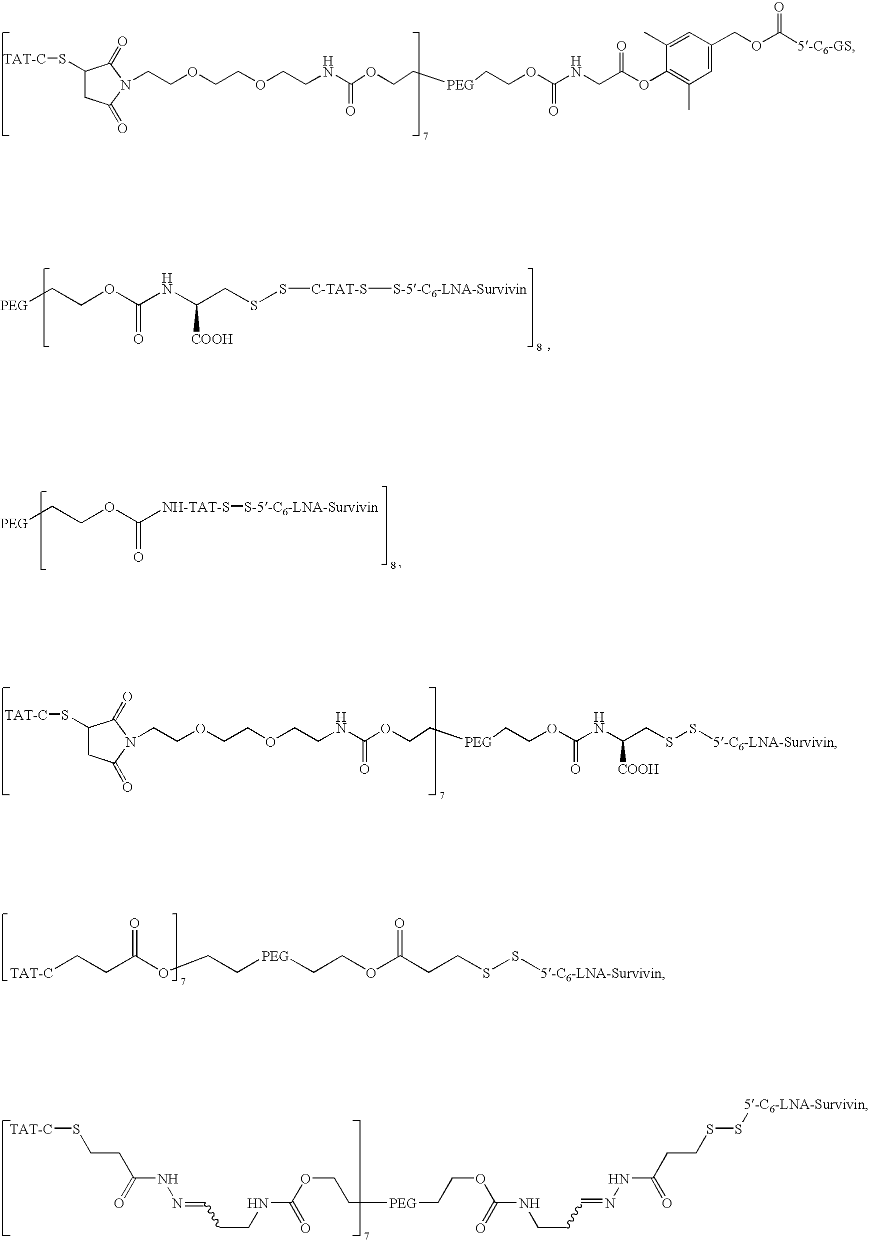

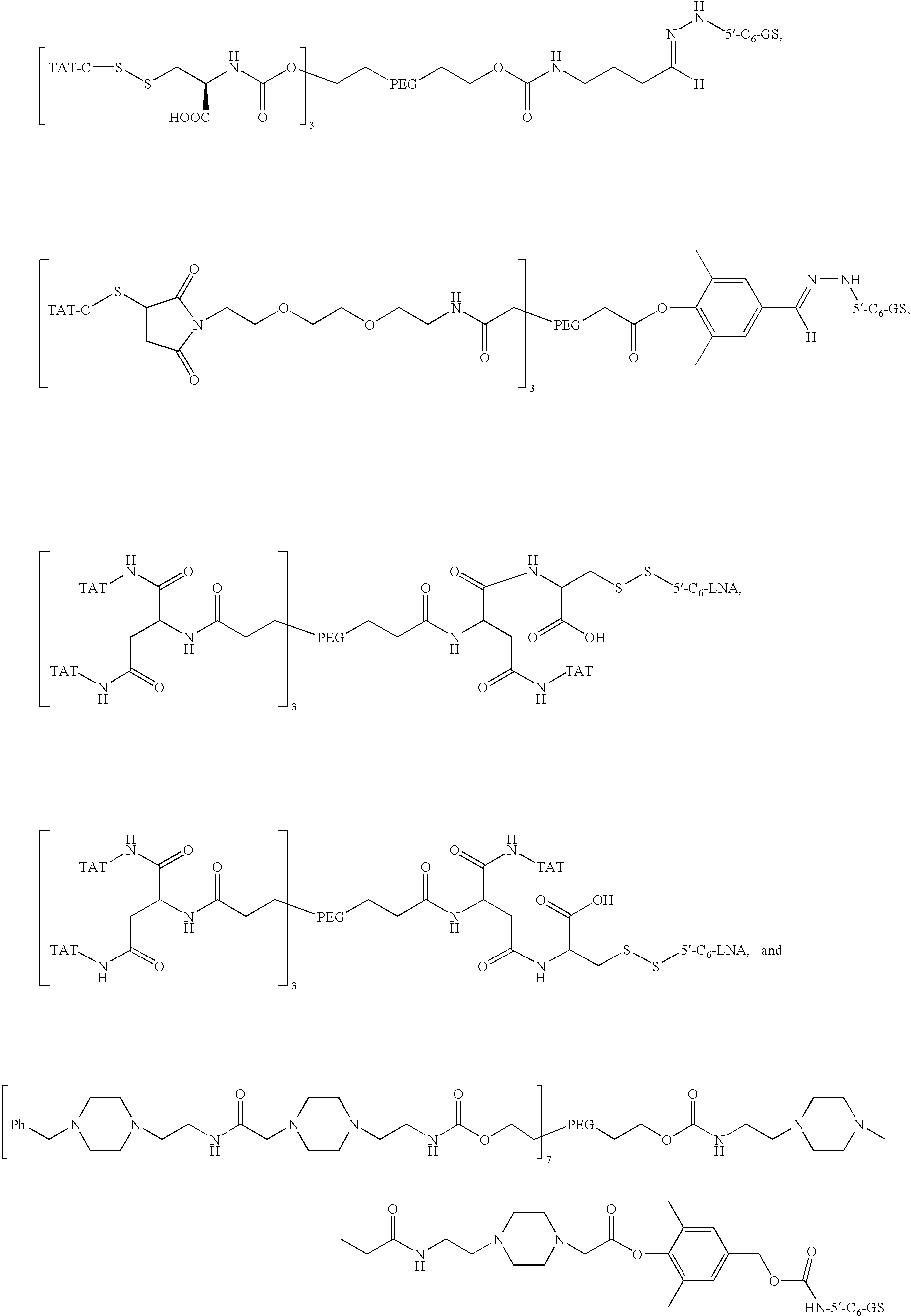

38. A method of treatment, comprising an effective amount of a compound of claim 1 to a mammal in need thereof.

39. A method of administering polynucleotides to mammalian cells, comprising delivering an effective amount of a compound of claim 1 to a cell requiring such treatment.
